(19) United States
(12) Patent Application Publication
(10) Pub. No.: US 2024/0238439 A1
(43) Pub. Date: Jul. 18, 2024

(54) HER3-BINDING ANTIBODY-DRUG CONJUGATE

(71) Applicant: Hummingbird Bioscience Pte. Ltd., Singapore (SG)

(72) Inventors: Suhail Ahmed Kabeer Rasheed, Singapore (SG); Dipti Thakkar, Singapore (SG); Shalini Paliwal, Singapore (SG); Wen Jie Chin, Singapore (SG); Akshaya Bansal, Singapore (SG); Benjamin Ayers, Singapore (SG); Cheng Theng Ng, Singapore (SG); Brendon Hanson, Singapore (SG); Piers Ingram, Singapore (SG); Jerome Boyd-Kirkup, Singapore (SG)

(21) Appl. No.: 18/403,476

(22) Filed: Jan. 3, 2024

Related U.S. Application Data
(60) Provisional application No. 63/459,061, filed on Apr. 13, 2023.
(60) Provisional application No. 63/436,730, filed on Jan. 3, 2023.

(51) Int. Cl. A61K 47/68; A61P 35/00; G01N 33/574

(52) U.S. Cl. CPC A61K 47/6851; A61K 47/68037; A61K 47/6889; A61P 35/00; G01N 33/5748; G01N 2333/82

(57) ABSTRACT

Methods of treating cancer are provided, as well as compositions including an antigen-binding molecule that binds to HER3, comprising (i) a HER3-binding moiety, and (ii) a linker-payload moiety comprising exatecan or a derivative thereof.

### CROSS-REFERENCE TO RELATED APPLICATIONS

This application claims the benefit of, and priority to, U.S. Provisional Patent Application 63/436,730 filed Jan. 3, 2023, and U.S. Provisional Patent Application 63/459,061 filed Apr. 13, 2023, the entire contents of each of which are incorporated by reference herein.

### SEQUENCE LISTING

This application contains a sequence listing having the filename 1959576-00081_Sequence_Listing.xml, which is 107 KB in size, and was created on Dec. 19, 2023. The entire content of this sequence listing is incorporated herein by reference.

### TECHNICAL FIELD

The present disclosure relates to molecular biology, more specifically antibody technology. The present disclosure also relates to methods of medical treatment and prophylaxis.

### BACKGROUND

Cancers remain the leading cause of deaths worldwide. Chemotherapies have good clinical benefits, but due to their low specificity they have very significant side effect and low therapeutic indices. More targeted therapies, such as monoclonal antibody therapies, show good specificity but response rates are smaller. Antibody-drug conjugates (ADCs) are a therapeutic modality that harness an antibody's target specificity to selectively deliver cytotoxic payloads to tumors and are proving increasingly effective in the clinic.

HER3 is a member of the Epidermal growth factor receptor (ERBB) family of tyrosine kinase receptors overexpressed in a broad range of tumors and is associated with disease progression and poor survival. For example, more than 50% of tumors show overexpression of HER3 in melanoma, cervical, lung, gastric, colorectal and ovarian cancers, while in breast, pancreatic and prostate cancers the overexpression ranged between 25-40% of tumors. In addition, recent studies have shown that HER3 expression is significantly induced in tumors during metastasis in colorectal cancers and during acquired resistance to the standard of care in lung cancers.

Patritumab-Deruxtecan (U3-1402; also referred to herein as Patritumab-DXd) is a HER3-targeted ADC for which clinical responses have been observed in a subset of EGFR-mutant and TKI-resistant lung cancers, and metastatic breast cancers (Jsnne et al., Cancer Discov. (2022) 12(1):74-89; Krop et al., J Clin Oncol (2022) 40 (suppl 16): abstr 1002). However, toxicities including severe interstitial lung disease (ILD) and high grade cytopenia have been observed (Kogawa et al., Journal of Clinical Oncology (2018) 36(15, suppl): 2512-2512).

### SUMMARY

In a first aspect, the present disclosure provides an antigen-binding molecule that binds to HER3, comprising (i) a HER3-binding moiety, and (ii) a linker-payload moiety comprising exatecan or a derivative thereof.

In some embodiments, the linker-payload moiety comprises structure (A):

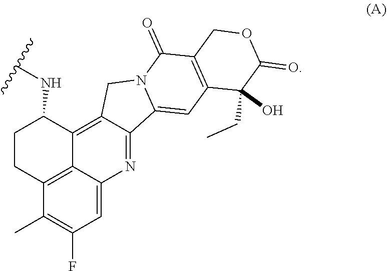

In some embodiments, the linker-payload moiety and the HER3-binding moiety are connected via a cleavable linker moiety.

In some embodiments, the antigen-binding molecule comprises a cleavable linker moiety comprising structure (B):

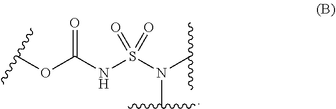 [SMILES: *OC(=O)NS(=O)(=O)N(*)*]

In some embodiments, the antigen-binding molecule comprises a cleavable linker moiety comprising a polyethylene glycol (PEG) moiety.

In some embodiments, the antigen-binding molecule comprises a cleavable linker moiety comprising structure (C):

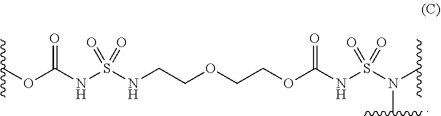 [SMILES: *OC(=O)NS(=O)(=O)NCCOCCOC(=O)NS(=O)(=O)N(*)*]

In some embodiments, antigen-binding molecule comprises a cleavable linker moiety comprising a p-aminobenzyl carbamate (PABC) group.

In some embodiments, the antigen-binding molecule comprises a cleavable linker moiety comprising structure (D):

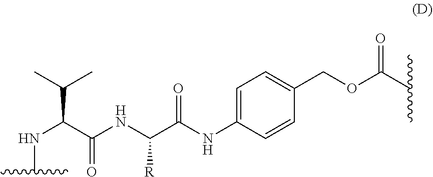

wherein R is selected from CH₃ and (CH₂)₃NHCOCNH₂.

In some embodiments, the antigen-binding molecule comprises structure (E):

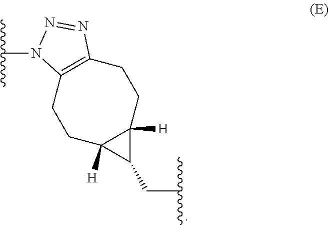 [SMILES: *C[C@H]1[C@H]2CCc3nnn(*)c3CC[C@@H]12]

In some embodiments, the antigen-binding molecule comprises SYNtecan E.

In some embodiments, the HER3-binding moiety comprises an Fc region, and wherein the linker-payload moiety is conjugated to the HER3-binding moiety at an azido group provided in a 6-azido-6-deoxy-N-acetylgalactosamineresidue of an N-glycan linked to N297 of a CH2 domain of the Fc region (EU numbering).

In some embodiments, the HER3-binding moiety binds to HER3 in the region shown in SEQ ID NO:77.

In some embodiments, the HER3-binding moiety comprises:
(i) a heavy chain variable (VH) region incorporating the following CDRs:
   HC-CDR1 having the amino acid sequence of SEQ ID NO:40
   HC-CDR2 having the amino acid sequence of SEQ ID NO:43
   HC-CDR3 having the amino acid sequence of SEQ ID NO:48; and
(ii) a light chain variable (VL) region incorporating the following CDRs:
   LC-CDR1 having the amino acid sequence of SEQ ID NO:66
   LC-CDR2 having the amino acid sequence of SEQ ID NO:69
   LC-CDR3 having the amino acid sequence of SEQ ID NO:74.

In some embodiments, the HER3-binding moiety comprises:
(i) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45; and
(ii) a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70.

In some embodiments, the antigen-binding moiety that binds to HER3 comprises:
a VH region comprising an amino acid sequence having at least 70% sequence identity to the amino acid sequence of SEQ ID NO:33; and
a VL region comprising an amino acid sequence having at least 70% sequence identity to the amino acid sequence of SEQ ID NO:58.

In some embodiments, the antigen-binding moiety that binds to HER3 comprises:
a polypeptide comprising, or consisting of, an amino acid sequence having at least 70% sequence identity to the amino acid sequence of SEQ ID NO:75; and
a polypeptide comprising, or consisting of, an amino acid sequence having at least 70% sequence identity to the amino acid sequence of SEQ ID NO:76.

In some embodiments, the antigen-binding moiety that binds to HER3 comprises:
a polypeptide comprising, or consisting of, an amino acid sequence having at least 70% sequence identity to the amino acid sequence of SEQ ID NO:80, 81, 82 or 91; and
a polypeptide comprising, or consisting of, an amino acid sequence having at least 70% sequence identity to the amino acid sequence of SEQ ID NO:76.

The present disclosure also provides a method for producing an antigen-binding molecule, comprising contacting a HER3-binding moiety comprising an azido moiety with a compound having structure (F):

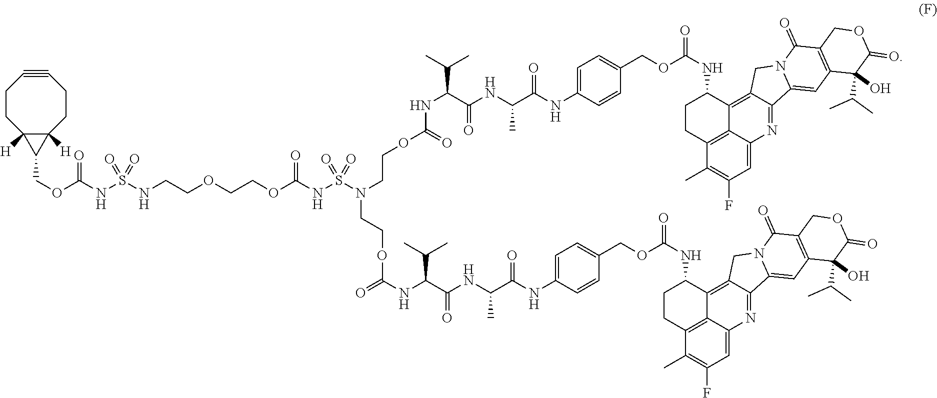

The present disclosure also provides an antigen-binding molecule obtained or obtainable by the method of the present disclosure.

The present disclosure also provides a composition comprising an antigen-binding molecule according to the present disclosure, and a pharmaceutically acceptable carrier, diluent, excipient or adjuvant.

The present disclosure also provides an antigen-binding molecule or composition according to the present disclosure, for use in a method of medical treatment or prophylaxis, or in a method of diagnosis or prognosis.

The present disclosure also provides an antigen-binding molecule or composition according to the present disclosure, for use in treating or preventing a cancer.

The present disclosure also provides the use of an antigen-binding molecule or composition according to the present disclosure, in the manufacture of a medicament for treating or preventing a cancer.

The present disclosure also provides a method of treating or preventing a cancer, comprising administering to a subject a therapeutically- or prophylactically-effective amount of an antigen-binding molecule or composition according to the present disclosure.

In some embodiments, the cancer is selected from: a cancer comprising cells expressing/overexpressing an EGFR family member, a cancer comprising cells expressing/overexpressing HER3, a cancer comprising cells having a mutation resulting in increased expression of a ligand for HER3, a cancer comprising cells having an NRG gene fusion, a solid tumor, a hematological cancer, a squamous cell cancer, breast cancer, breast carcinoma, breast invasive carcinoma, ductal carcinoma, metastatic breast cancer, triple-negative breast cancer, HER2-positive breast cancer, HER2-negative breast cancer, hormone receptor-positive breast cancer, HER2-negative/hormone receptor-positive breast cancer, gastric cancer, gastric carcinoma, gastric adenocarcinoma, gastrointestinal adenocarcinoma, colorectal cancer, metastatic colorectal cancer, colon cancer, colorectal carcinoma, colorectal adenocarcinoma, colon adenocarcinoma, head and neck cancer, head and neck squamous cell carcinoma (HNSCC), lung cancer, non-small cell lung cancer, lung adenocarcinoma, invasive mucinous lung adenocarcinoma, lung squamous cell carcinoma (LUSC), ovarian cancer, ovarian carcinoma, ovarian serous adenocarcinoma, ovarian serous cystadenocarcinoma, fallopian tube cancer, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, oral cavity cancer, oropharyngeal cancer, esophageal cancer, esophageal squamous cell carcinoma (ESCC), esophageal adenocarcinoma, liver cancer, hepatocellular carcinoma, cholangiocarcinoma, gallbladder cancer, biliary tract cancer, uterine cancer, endometrial cancer, uterine corpus endometrial carcinoma, uterine carcinosarcoma, thyroid cancer, thyroid carcinoma, pheochromocytoma, paraganglioma, bladder cancer, bladder urothelial carcinoma, prostate cancer, prostate adenocarcinoma, castration-resistant prostate cancer, metastatic prostate cancer, metastatic castration-resistant prostate cancer, retinoblastoma, sarcoma, soft tissue sarcoma, peritoneal cancer, thymoma, neuroendocrine tumor, neuroendocrine tumor of the nasopharynx and homologous recombination deficiency (HRD) cancer.

The present disclosure also provides the use of an antigen-binding molecule or composition according to the present disclosure to deplete or increase killing of cells expressing HER3.

The present disclosure also provides an in vitro complex, optionally isolated, comprising an antigen-binding molecule according to the present disclosure bound to HER3.

The present disclosure also provides a method for detecting HER3 in a sample, comprising contacting a sample containing, or suspected to contain, HER3 with an antigen-binding molecule according to the present disclosure, and detecting the formation of a complex of the antigen-binding molecule with HER3.

The present disclosure also provides a method of selecting or stratifying a subject for treatment with a HER3-targeted agent, the method comprising contacting, in vitro, a sample from the subject with an antigen-binding molecule according to the present disclosure, and detecting the formation of a complex of the antigen-binding molecule with HER3.

The present disclosure also provides the use of an antigen-binding molecule according to the present disclosure as an in vitro or in vivo diagnostic or prognostic agent.

### BRIEF DESCRIPTION OF THE FIGURES

Embodiments and experiments illustrating the principles of the present disclosure will now be discussed with reference to the accompanying figures.

FIGS. 1A to 1F. Graphs and table showing binding of HER3-binding molecules to cells of different human cancer cell lines. (1A) to (1E) show binding of different molecules to (1A) HCT116 cells (1B) DU145 cells (1C) H358 cells, (1D) T47D cells and (1E) OVCAR8 cells. (1F) Table showing EC₅₀ values for binding of the indicated HER3-binding molecules to the indicated cell types, as derived from the binding curves.

FIGS. 2A to 2D. Graphs showing binding of HER3-binding molecules to cells of different human cancer cell lines, in the presence of increasing concentrations of NRG1. The y-axis shows relative mean fluorescence intensity (MFI) fold change compared to cells not treated with NRG1. (2A) to (2D) show binding of different molecules to (2A) OVCAR8 cells (2B) T47D cells (2C) H358 cells, and (2D) DU145 cells.

FIGS. 3A and 3B. Graphs and table showing binding of HER3-binding molecules to Fcγ receptors, as determined by surface plasmon resonance analysis. (3A) shows binding of a human IgG1 isotype-matched, positive control (IgG1 control), human IgG1 comprising Fc-silencing mutations (Fc Silenced), Patritumab-DXd, 501b.S8D4 and 501c.S8D4 to human FCγRIA (CD64), human FCγRIIA (CD32a) and human FCγRIIIA (CD16a). The y-axis shows relative resonance units (RU), which is directly proportional to the binding of the molecules to the Fcγ receptors tested, and the x-axis shows increasing concentration of the relevant test molecule. (3B) Table showing values for binding kinetic constants and calculated affinities derived from the binding data.

FIGS. 4A and 4B. Graphs and tables showing inhibition of proliferation of different human cancer cell lines having varying levels of basal HER3 expression by HER3-binding molecules. (4A) and (4B) show inhibition of proliferation of (4A) T47D cells and (4B) HCT116 cells. The IC₅₀ values for each of the test articles are provided in the tables below the respective graph, wherever applicable (IC₅₀ values could not be HCT116 cells).

FIGS. 5A and 5B. Graphs and tables showing inhibition of proliferation of T47D cells by HER3-binding molecules, in the presence of NRG1. (5A) and (5B) show inhibition of proliferation of T47D cells in the presence of the indicated molecules, in absence or presence of NRG1 (100 ng/ml). The IC₅₀ values for each of the test articles are provided in the tables below the respective graphs.

FIG. 6. Graph showing the percentage of species having a drug-to-antibody ratio of 4 (DAR4) following incubation of the indicated HER3-binding molecules in mouse serum at 37° C. for the indicated number of days.

FIG. 7. Graph showing tumor growth inhibition of mice in a H358 cell-derived xenograft model, following treatment with HER3-binding molecules or the vehicle control is shown. Tumor volume over time is shown in the graph.

FIGS. 8A and 8B. Graphs showing tumor growth inhibition of mice in a T47D cell-derived xenograft model, following treatment with different doses of HER3-binding molecules or the vehicle control. (8A) and (8B) show tumor volume over time.

FIGS. 9A to 9F. Bar charts showing levels of red blood cell indices and anemic parameters in blood samples taken from rats following administration of the indicated molecules. (9A) shows red blood cell count, (9B) shows hemoglobin concentration, (9C) shows hematocrit percentage, (9D) shows mean corpuscular volume (MCV), (9E) shows mean corpuscular hemoglobin (MCH) volume, and (9F) shows mean corpuscular hemoglobin concentration (MCHC). The dotted lines indicate the baseline normal range of these indices in healthy female rats as published by Charles River (Clinical Pathology Data for North American CD® Rat Colonies for January 2006—December 2011).

FIGS. 10A to 10E. Bar charts showing levels of white blood cell indices and platelets in blood samples taken from rats following administration of the indicated molecules, at 3 days after dose 1 (day 4) and 3 days after dose 2 (day 11). (10A) shows white blood cell count, (10B) shows lymphocyte count, (10C) shows monocyte count, (10D) shows neutrophil count, and (10E) shows platelet count. The dotted lines indicate the baseline normal range of these indices in healthy female rats as published by Charles River (Clinical Pathology Data for North American CD® Rat Colonies for January 2006—December 2011).

FIGS. 11A to 11F. Bar charts showing levels of liver, kidney and pancreatic indices in blood samples taken from rats following administration of the indicated molecules, at 3 days after dose 1 (day 4) and 3 days after dose 2 (day 11). (11A) shows alanine aminotransferase (ALT) level, (11B) shows aspartate aminotransferase (AST) level, (11C) shows albumin level, (11D) shows total protein level, (11E) shows creatinine level, and (11F) shows glucose level. The dotted lines indicate the baseline normal range of these indices in healthy female rats as published by Charles River (Clinical Pathology Data for North American CD® Rat Colonies for January 2006—December 2011).

FIGS. 12A to 12D. Bar charts showing levels of electrolytes in blood samples taken from rats following administration of the indicated molecules, at 3 days after dose 1 (day 4) and 3 days after dose 2 (day 11). (12A) shows sodium level, (12B) shows potassium level, (12C) phosphate level, (12D) shows calcium level. The dotted lines indicate the baseline normal range of these indices in healthy female rats as published by Charles River (Clinical Pathology Data for North American CD® Rat Colonies for January 2006—December 2011).

FIG. 13. Fluorescence images of APC-labelled 501c.S8D4 incubated with T47D cells at 37° C. for 0.5, 4 and 16 h to measure internalization and trafficking to the lysosomes. Cells incubated at 4° C. for 1 h provided a negative control.

FIG. 14. Graph showing internalization of Fab-fluor-labelled 501c.S8D4 into lysosomes of T47D cells using live cell imaging.

FIG. 15. Graph showing macropinocytosis-dependent uptake of Fab-fluor-labelled 501c.S8D4 or patritumab-DXd by human megakaryocytes. p values, ***<0.001.

FIG. 16. Graph showing Fc-mediated binding of IgG-DXd (positive control), 501c.S8D4 or patritumab-DXd to human PBMCs at concentrations of 30 μg/ml, 10 μg/ml, or 3 μg/ml. p values, ** <0.01, ***<0.001.

### DESCRIPTION

The present disclosure relates to antigen-binding molecules comprising a HER3-binding moiety and an exatecan payload moiety.

In the experimental examples of the present disclosure, the inventors demonstrate that such antigen-binding molecules are provided with unexpected and advantageous properties relative to known anti-HER3 antibody-drug conjugates.

In particular, the antigen-binding molecules of the present disclosure are demonstrated herein to have similar or improved anti-cancer activity in vitro and in vivo as compared to the known anti-HER3 antibody-drug conjugate Patritumab-DXd, while having a much more favourable toxicological profile.

The similar/improved anti-cancer activity over Patritumab-DXd is especially unexpected due to the antigen-binding molecules of the disclosure having a mean drug-to-antibody ratio (DAR) of 4, while the mean DAR for Patritumab-DXd is 8.

### Antigen-Binding Moieties

An ‘antigen-binding molecule’ refers to a molecule that binds to a given target antigen. Antigen-binding molecules comprise an antigen-binding moiety through which the antigen-binding molecule binds to its target antigen. The antigen-binding molecules of the present disclosure comprise an antigen-binding moiety that binds to HER3 (i.e. a HER3-binding moiety).

Antigen-binding moieties may comprise, or may be derived from, antibodies (i.e. immunoglobulins (Igs)) and antigen-binding fragments of antibodies. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH, etc.). Antigen-binding fragments of antibodies include e.g. Fv, Fab, F(ab′)₂ and F(ab′) fragments.

Antigen-binding moieties also include target antigen-binding aptamers, e.g. a nucleic acid aptamers (reviewed, for example, in Zhou and Rossi, Nat Rev Drug Discov. (2017) 16(3):181-202). In some embodiments, an antigen-binding moiety comprises or consists of an antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

In some embodiments, an antigen-binding moiety comprises, or consists of, the antigen-binding region of an antibody (e.g. an antigen-binding fragment of an antibody). Antigen-binding moieties of the antigen-binding molecules of the present disclosure preferably comprise the antibody heavy chain variable region (VH) and the antibody light chain variable region (VL) of an antibody that binds to the target antigen. The antigen-binding domain formed by a VH and a VL may also be referred to herein as an Fv region.

In some embodiments, the antigen-binding moiety is or comprises the Fv (e.g. provided as an scFv) of an antibody. In some embodiments, the antigen-binding moiety is or comprises the Fab region of an antibody. In some embodiments, the antigen-binding moiety is or comprises the whole antibody (i.e. comprising variable and constant regions).

An antigen-binding moiety may be, or may comprise, an antigen-binding polypeptide, or an antigen-binding polypeptide complex. An antigen-binding moiety may comprise more than one polypeptide which together form an antigen-binding moiety. The polypeptides may associate covalently or non-covalently. In some embodiments, the polypeptides form part of a larger polypeptide comprising the polypeptides (e.g. in the case of scFv comprising VH and VL, or in the case of scFab comprising VH-CH1 and VL-CL).

An antigen-binding moiety may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding moieties of the present disclosure may be designed and prepared using the sequences of monoclonal antibodies (mAbs) capable of binding to a given target antigen (e.g. HER3). Antigen-binding regions of antibodies, such as single chain variable fragment (scFv), Fab and F(ab′)₂ fragments may also be used/provided. An ‘antigen-binding region’ is any fragment of an antibody that binds to the target for which the given antibody is specific.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5^{th} Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77. In preferred embodiments, the CDRs and FRs of antigen-binding molecules referred to herein are defined according to the IMGT information system.

In some embodiments, an antigen-binding molecule according to the present disclosure comprises, or consists of, an Fv region that binds to HER3. In some embodiments, the VH and VL regions of the Fv are provided as single polypeptide joined by a linker sequence, i.e. a single chain Fv (scFv).

The VL and light chain constant (CL) region, and the VH region and heavy chain constant 1 (CH1) region of an antigen-binding region of an antibody together constitute the Fab region. In some embodiments, the antigen-binding molecule comprises a Fab region comprising a VH, a CH1, a VL and a CL (e.g. Cκ or Cλ).

In some embodiments, the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments, the Fab region comprises a polypeptide comprising a VH and a CL (e.g. a VH-CL fusion polypeptide) and a polypeptide comprising a VL and a CH1 (e.g. a VL-CH1 fusion polypeptide); that is, in some embodiments, the Fab region is a CrossFab region. In some embodiments, the VH, CH1, VL and CL regions of the Fab or CrossFab are provided as single polypeptide joined by linker regions, i.e. as a single chain Fab (scFab) or a single chain CrossFab (scCrossFab).

In some embodiments, an antigen-binding molecule described herein comprises, or consists of, a whole antibody which binds to HER3. As used herein, ‘whole antibody’ refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are −150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the antigen-binding molecule comprises, or consists of, an IgG (e.g. IgG1, IgG2, IgG3, IgG4), IgA (e.g. IgA1, IgA2), IgD, IgE, or IgM which binds to HER3.

In some embodiments described herein, one or more amino acids of an amino acid sequence referred to herein (e.g. an amino acid sequence of an antigen-binding molecule, e.g. an amino acid sequence of a CDR or VH/VL region) are substituted with another amino acid. A substitution comprises substitution of an amino acid residue with a non-identical ‘replacement’ amino acid residue. A replacement amino acid residue of a substitution according to the present disclosure may be a naturally-occurring amino acid residue (i.e. encoded by the genetic code) which is non-identical to the amino acid residue at the relevant position of the equivalent, unsubstituted amino acid sequence, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (lie): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

In some embodiments, a substitution may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, Trp, |
| | | Tyr, Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, Ile, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | | Side-chain charge |
|---|---|---|
| Amino Acid | Side-chain polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target binding) of the antigen-binding molecule comprising the substitution as compared to the equivalent unsubstituted molecule.

The antigen-binding molecules of the present disclosure comprise an antigen-binding moiety that binds to HER3.

In some embodiments, the antigen-binding moiety comprises the CDRs of an antigen-binding moiety which is capable of binding to HER3. In some embodiments, the antigen-binding moiety comprises the FRs of an antigen-binding moiety which is capable of binding to HER3. In some embodiments, the antigen-binding moiety comprises the CDRs and the FRs of an antibody that is capable of binding to HER3. That is, in some embodiments the antigen-binding moiety comprises the VH region and the VL region of an antibody that is capable of binding to HER3.

In some embodiments, an antigen-binding moiety which is capable of binding to HER3 according to the present disclosure may be, or may be derived from, a HER3-binding antibody selected from: an antibody described in WO 2019/185878 A1 (which is hereby incorporated by reference in its entirety), 10D1F (described e.g. in WO 2019/185878 A1), seribantumab (also known as MM-121, described e.g. in Schoeberl et al., Sci. Signal. (2009) 2(77): ra31; DrugBank Acc. No. DB11857), elgemtumab (also known as LJM-716, described e.g. in Garner et al., Cancer Res (2013) 73: 6024-6035; DrugBank Acc. No. DB15430), Patritumab (also known as U-1287 and AMG-888, described e.g. in Shimizu et al. Cancer Chemother Pharmacol. (2017) 79(3):489-495; DrugBank Acc. No. DB12090), GSK2849330 (described e.g. in Clarke et al., Eur J Cancer. (2014) 50:98-9), lumretuzumab (also known as RG7116 and RO-5479599, described e.g. in Mirschberger et al. Cancer Research (2013) 73(16) 5183-5194; DrugBank Acc. No. DB12683), CDX-3379 (also known as KTN3379, described e.g. in Lee et al., Proc Natl Acad Sci USA. 2015 Oct. 27; 112(43):13225), AV-203 (also known as CAN-017, described e.g. in Meetze et al., Eur J Cancer 2012; 48:126), barecetamab (also known as ISU104, described e.g. in Kim et al., Cancer Res (2018) 78(13 Suppl):Abstract #830), TK-A3, TK-A4 (described e.g. in Malm et al., Mabs (2016) 8:1195-209), MP-EV20 (described e.g. in Sala et al., Transl. Oncol. (2013) 6:676-84), 1A5-3D4 (described e.g. in Wang et al., Cancer Lett (2016) 380:20-30), 9F7-F11, 16D3-C1 (described e.g. in Lazrek et al., Neoplasia (2013) 15:335-47), NG33, A5, F4 (described e.g. in Gaborit et al., PNAS USA (2015) 112:839-44), huHER3-8 (described e.g. in Kugel et al., Cancer Res. (2014) 74:4122-32), REGN1400 (described e.g. in Zhang et al., Mol Cancer Ther (2014) 13:1345-1355) and zenocutuzumab (also known as MCLA-128, described e.g. in de Vries Schultink et al., Clin Pharmacokinet. (2020) 59: 875-884; DrugBank Acc. No. DB15559). In some embodiments, the antigen-binding moiety is, or is derived from, 10D1F.

In some embodiments, the antigen-binding moiety binds to the extracellular region of HER3 (e.g. the region shown in SEQ ID NO:9). In some embodiments, the antigen-binding moiety binds to subdomain II of the extracellular region of HER3 (e.g. the region shown in SEQ ID NO:16).

In some embodiments, the antigen-binding moiety binds to the region of HER3 shown in SEQ ID NO:77. In some embodiments the antigen-binding moiety contacts one or more amino acid residues of the region of HER3 shown in SEQ ID NO:77. In some embodiments, the antigen-binding moiety binds to the regions of HER3 shown in SEQ ID NOs:78 and 79. In some embodiments the antigen-binding moiety contacts one or more amino acid residues of the regions of HER3 shown in SEQ ID NOs:78 and 79. In some embodiments, the antigen-binding moiety binds to the region of HER3 shown in SEQ ID NO:78. In some embodiments the antigen-binding moiety contacts one or more amino acid residues of the region of HER3 shown in SEQ ID NO:78. In some embodiments, the antigen-binding moiety binds to the region of HER3 shown in SEQ ID NO:79. In some embodiments the antigen-binding moiety contacts one or more amino acid residues of the region of HER3 shown in SEQ ID NO:79.

In some embodiments, the antigen-binding moiety does not bind to the region of HER3 corresponding to positions 260 to 279 of SEQ ID NO:1. In some embodiments the antigen-binding moiety does not contact an amino acid residue of the region of HER3 corresponding to positions 260 to 279 of SEQ ID NO:1.

The region of a peptide/polypeptide to which an antibody/antigen-binding moiety binds can be determined by the skilled person using various methods well known in the art, including X-ray crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In some embodiments the antigen-binding moiety is capable of binding the same region of HER3, or an overlapping region of HER3, to the region of HER3 which is bound by an antigen-binding molecule comprising the VH and VL sequences of one of antibody clones 10D1_c89, 10D1, 10D1_c75, 10D1_c76, 10D1_c77, 10D1_c78v1, 10D1_c78v2, 10D1_11B, 10D1_c85v1, 10D1_c85v2, 10D1_c85o1, 10D1_c85o2, 10D1_c87, 10D1_c90, 10D1_c91, 10D1_c92 and 10D1_c93 described herein. In some embodiments the antigen-binding moiety is capable of binding the same region of HER3, or an overlapping region of HER3, to the region of HER3 which is bound by an antigen-binding molecule comprising the VH and VL sequences of antibody clone 10D1_c89.

In some embodiments, the antigen-binding moiety is capable of binding to a polypeptide comprising, or consisting of, the amino acid sequence of one of SEQ ID NOs:1, 3, 4, 6 or 8. In some embodiments, the antigen-binding moiety is capable of binding to a polypeptide comprising, or consisting of, the amino acid sequence of SEQ ID NO:9. In some embodiments, the antigen-binding moiety is capable of binding to a polypeptide comprising, or consisting of, the amino acid sequence of SEQ ID NO:16. In some embodiments, the antigen-binding moiety is capable of binding to a peptide/polypeptide comprising, or consisting of, the amino acid sequence of SEQ ID NO:77. In some embodiments, the antigen-binding moiety is capable of binding to a peptide/polypeptide comprising, or consisting of, the amino acid sequences of SEQ ID NOs:78 and 79. In some embodiments, the antigen-binding moiety is capable of binding to a peptide/polypeptide comprising, or consisting of, the amino acid sequence of SEQ ID NO:78. In some embodiments, the antigen-binding moiety is capable of binding to a peptide/polypeptide comprising, or consisting of, the amino acid sequence of SEQ ID NO:79.

In some embodiments, the antigen-binding moiety is not capable of binding to a peptide consisting of the amino acid sequence corresponding to positions 260 to 279 of SEQ ID NO:1.

The ability of an antigen-binding moiety to bind to a given peptide/polypeptide can be analysed by methods well known to the skilled person, including analysis by ELISA, immunoblot (e.g. western blot), immunoprecipitation, Surface Plasmon Resonance (SPR; see e.g. Hearty et al., Methods Mol Biol (2012)907:411-442) or Bio-Layer Interferometry (see e.g. Lad et al., (2015) J Biomol Screen 20(4): 498-507).

In embodiments where the antigen binding moiety is capable of binding to a peptide/polypeptide comprising a reference amino acid sequence, the peptide/polypeptide may comprise one or more additional amino acids at one or both ends of the reference amino acid sequence. In some embodiments the peptide/polypeptide comprises e.g. 1-5, 1-10, 1-20, 1-30, 1-40, 1-50, 5-10, 5-20, 5-30, 5-40, 5-50, 10-20, 10-30, 10-40, 10-50, 20-30, 20-40 or 20-50 additional amino acids at one or both ends of the reference amino acid sequence.

In some embodiments the additional amino acid(s) provided at one or both ends (i.e. the N-terminal and C-terminal ends) of the reference sequence correspond to the positions at the ends of the reference sequence in the context of the amino acid sequence of HER3.

In some embodiments the antigen-binding moiety is capable of binding to a peptide/polypeptide which is bound by an antibody comprising the VH and VL sequences of one of antibody clones 10D1_c89, 10D1, 10D1_c75, 10D1_c76, 10D1_c77, 10D1_c78v1, 10D1_c78v2, 10D1_11B, 10D1_c85v1, 10D1_c85v2, 10D1_c85o1, 10D1_c85o2, 10D1_c87, 10D1_c90, 10D1_c91, 10D1_c92 and 10D1_c93 described herein. In some embodiments the antigen-binding moiety is capable of binding to a peptide/polypeptide which is bound by an antibody comprising the VH and VL sequences of antibody clone 10D1_c89.

In some embodiments, the antigen-binding moiety comprises the CDRs of, or comprises the VH and VL of, a HER3-binding antibody clone selected from 10D1_c89, 10D1, 10D1_c75, 10D1_c76, 10D1_c77, 10D1_c78v1, 10D1_c78v2, 10D1_11B, 10D1_c85v1, 10D1_c85v2, 10D1_c85o1, 10D1_c85o2, 10D1_c87, 10D1_c90, 10D1_c91, 10D1_c92 and 10D1_c93.

In some embodiments, the antigen-binding moiety comprises:
(4) a VH region incorporating the following CDRs:
   HC-CDR1 having the amino acid sequence of SEQ ID NO:40
   HC-CDR2 having the amino acid sequence of SEQ ID NO:43
   HC-CDR3 having the amino acid sequence of SEQ ID NO:48,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:66
   LC-CDR2 having the amino acid sequence of SEQ ID NO:69
   LC-CDR3 having the amino acid sequence of SEQ ID NO:74;
   or a variant thereof in which one or two or three amino acids in one or more of LC-CDR1, LC-CDR2 or LC-CDR3 are substituted with another amino acid.
(2) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(3) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:64
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(4) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs: LC-CDR1 having the amino acid sequence of SEQ ID NO:65 LC-CDR2 having the amino acid sequence of SEQ ID NO:67 LC-CDR3 having the amino acid sequence of SEQ ID NO:71;
   or a variant thereof in which one or two or three amino acids in one or more of LC-CDR1, LC-CDR2 or LC-CDR3 are substituted with another amino acid.
(5) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(6) a VH region incorporating the following CDRs:
   HC-CDR1 having the amino acid sequence of SEQ ID NO:39
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(7) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
or a variant thereof in which one or two or three amino acids in one or more of LC-CDR1, LC-CDR2 or LC-CDR3 are substituted with another amino acid.
(8) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:46,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(9) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:47,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(10) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:72;
   or a variant thereof in which one or two or three amino acids in one or more of LC-CDR1, LC-CDR2 or LC-CDR3 are substituted with another amino acid.
(11) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:73;
   or a variant thereof in which one or two or three amino acids in one or more of LC-CDR1, LC-CDR2 or LC-CDR3 are substituted with another amino acid.

In some embodiments, the antigen-binding moiety comprises:
(12) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:21; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:49.
(13) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:50.
(14) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:51.
(15) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:24; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.
(16) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:25; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(17) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(18) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(19) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:28; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(20) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:29; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(21) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:55.
(22) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:56.
(23) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:57.
(24) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:33; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:58.
(25) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:34; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:59.
(26) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:35; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:60.
(27) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:36; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:61.
(28) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:37; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:62.

In some embodiments, an antigen-binding moiety comprises, or consists of:
(29) (i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:75; and
   (ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:76.
(30) (i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:80; and
   (ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:76.
(31) (i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:81; and
   (ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:76.
(32) (i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:82; and
   (ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:76.
(33) (i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:91; and
   (ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:76.

In some embodiments, the antigen-binding moieties of the present disclosure comprise an Fc region.

As used herein, an ‘Fc region’ refers to a polypeptide complex formed by interaction between two polypeptides, each polypeptide comprising the CH2-CH3 region of an immunoglobulin (Ig) heavy chain constant sequence.

Herein, a ‘CH2 domain’ refers to an amino acid sequence corresponding to the CH2 domain of an immunoglobulin (Ig). The CH2 domain is the region of an Ig formed by positions 231 to 340 of the immunoglobulin constant domain, according to the EU numbering system described in Edelman et al., Proc Natl Acad Sci USA (1969) 63(1): 78-85. A ‘CH3 domain’ refers to an amino acid sequence corresponding to the CH3 domain of an immunoglobulin (Ig). The CH3 domain is the region of an Ig formed by positions 341 to 447 of the immunoglobulin constant domain, according to the EU numbering system described in Edelman et al., Proc Natl Acad Sci USA (1969) 63(1): 78-85. A ‘CH2-CH3 region’ refers to an amino acid sequence corresponding to the CH2 and CH3 domains of an immunoglobulin (Ig). The CH2-CH3 region is the region of an Ig formed by positions 231 to 447 of the immunoglobulin constant domain, according to the EU numbering system described in Edelman et al., Proc Natl Acad Sci USA (1969) 63(1): 78-85.

In some embodiments, a CH2 domain, CH3 domain and/or a CH2-CH3 region according to the present disclosure corresponds to the CH2 domain/CH3 domain/CH2-CH3 region of an IgG (e.g. IgG1, IgG2, IgG3, IgG4), IgA (e.g. IgA1, IgA2), IgD, IgE or IgM. In some embodiments, the CH2 domain, CH3 domain and/or a CH2-CH3 region corresponds to the CH2 domain/CH3 domain/CH2-CH3 region of a human IgG (e.g. hIgG1, hIgG2, hIgG3, hIgG4), hIgA (e.g. hIgA1, hIgA2), hIgD, hIgE or hIgM. In some embodiments, the CH2 domain, CH3 domain and/or a CH2-CH3 region corresponds to the CH2 domain/CH3 domain/CH2-CH3 region of a human IgG1 allotype (e.g. G1 m1, G1m2, G1m3 or G1ml7).

Fc regions provide for interaction with Fc receptors and other molecules of the immune system to bring about functional effects. Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

The CH2-CH3 region sequence of the human IgG1 G1 m1 allotype is shown in SEQ ID NO:83. The CH2-CH3 region sequence of the human IgG1 G1m3 allotype is shown in SEQ ID NO:84. The CH2-CH3 region sequence of human IgG2 is shown in SEQ ID NO:85. The CH2-CH3 region sequence of human IgG3 is shown in SEQ ID NO:86. The CH2-CH3 region sequence of human IgG4 is shown in SEQ ID NO:87.

In some embodiments, an Fc region according to the present disclosure comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or ≥100% sequence identity, to the amino acid sequence of SEQ ID NO:83, 84, 85, 86 or 87. In some embodiments, a reference Fc region according to the present disclosure comprises two polypeptides, wherein each polypeptide comprises a CH2-CH3 region comprising or consisting of the amino acid sequence of SEQ ID NO:83, 84, 85, 86 or 87.

Modifications to antibody Fc regions that influence Fc-mediated functions are known in the art, such as those described e.g. in Wang et al., Protein Cell (2018) 9(1):63-73, which is hereby incorporated by reference in its entirety. Exemplary Fc region modifications known to influence antibody effector function are summarised in Table 1 of Wang et al., Protein Cell (2018) 9(1):63-73. In some embodiments, the antigen-binding molecule of the present disclosure comprises an Fc region comprising modification to increase or reduce an Fc-mediated function as compared to an antigen-binding molecule comprising the corresponding unmodified Fc region. Where an Fc region/CH2/CH3 is described as comprising modification(s) ‘corresponding to’ reference substitution(s), equivalent substitution(s) in the homologous Fc/CH2/CH3 are contemplated. By way of illustration, L234A/L235A substitutions in human IgG1 (numbered according to the EU numbering system as described in Kabat et al., Sequences of Proteins of Immunological Interest, 5^{th} Ed. Public Health Service, National Institutes of Health, Bethesda, MD, 1991) correspond to L to A substitutions at positions 117 and 118 of the mouse Ig gamma-2A chain C region (UniProtKB: P01863-1, v1). Where an Fc region is described as comprising a modification, the modification may be present in one or both of the polypeptide chains which together form the Fc region.

In some embodiments, the antigen-binding molecule of the present disclosure comprises an Fc region comprising modification. In some embodiments, the antigen-binding molecule of the present disclosure comprises an Fc region comprising modification in one or more of the CH2 and/or CH3 regions.

In some embodiments, the Fc region comprises modification to increase an Fc-mediated function. In some embodiments, the Fc region comprises modification to increase ADCC. In some embodiments, the Fc region comprises modification to increase ADCP. In some embodiments, the Fc region comprises modification to increase CDC. An antigen-binding molecule comprising an Fc region comprising modification to increase an Fc-mediated function (e.g. ADCC, ADCP, CDC) induces an increased level of the relevant effector function as compared to an antigen-binding molecule comprising the corresponding unmodified Fc region. In some embodiments, the Fc region comprises modification to increase binding to an Fc receptor. In some embodiments, the Fc region comprises modification to increase binding to an Fcγ receptor. In some embodiments, the Fc region comprises modification to increase binding to one or more of FcγRI, FcγRIIa, FcγRIIb, FcγRIIc, FcγRIIIa and FcγRIIIb. In some embodiments, the Fc region comprises modification to increase binding to FcγRIIIa. In some embodiments, the Fc region comprises modification to increase binding to FcγRIIa. In some embodiments, the Fc region comprises modification to increase binding to FcγRIIb. In some embodiments, the Fc region comprises modification to increase binding to FcRn. In some embodiments, the Fc region comprises modification to increase binding to a complement protein. In some embodiments, the Fc region comprises modification to increase binding to C1q. In some embodiments, the Fc region comprises modification to promote hexamerisation of the antigen-binding molecule. In some embodiments, the Fc region comprises modification to increase antigen-binding molecule half-life. In some embodiments, the Fc region comprises modification to increase co-engagement.

In some embodiments, the Fc region comprises modification corresponding to the combination of substitutions F243L/R292P/Y300LV3051/P396L as described in Stavenhagen et al. Cancer Res. (2007)67:8882-8890. In some embodiments, the Fc region comprises modification corresponding to the combination of substitutions S239D/1332E or S239D/1332E/A330L as described in Lazar et al., Proc Natl Acad Sci USA. (2006)103:4005-4010. In some embodiments, the Fc region comprises modification corresponding to the combination of substitutions S298A/E333A/K334A as described in Shields et al., J Biol Chem. (2001) 276:6591-6604. In some embodiments, the Fc region comprises modification to one of heavy chain polypeptides corresponding to the combination of substitutions L234Y/L235Q/G236W/S239M/H268D/D270E/S298A, and modification to the other heavy chain polypeptide corresponding to the combination of substitutions D270E/K326D/A330M/K334E, as described in Mimoto et al., Mabs. (2013): 5:229-236. In some embodiments, the Fc region comprises modification corresponding to the combination of substitutions G236A/S239D/1332E as described in Richards et al., Mol Cancer Ther. (2008) 7:2517-2527.

In some embodiments, the Fc region comprises modification corresponding to the combination of substitutions K326W/E333S as described in Idusogie et al. J Immunol. (2001) 166(4):2571-5. In some embodiments, the Fc region comprises modification corresponding to the combination of substitutions S267E/H268F/S324T as described in Moore et al. Mabs. (2010) 2(2):181-9. In some embodiments, the Fc region comprises modification corresponding to the combination of substitutions described in Natsume et al., Cancer Res. (2008) 68(10):3863-72. In some embodiments, the Fc region comprises modification corresponding to the combination of substitutions E345R/E430G/S440Y as described in Diebolder et al. Science (2014) 343(6176):1260-3.

In some embodiments, the Fc region comprises modification corresponding to the combination of substitutions M252Y/S254T/T256E as described in Dall'Acqua et al. J Immunol. (2002) 169:5171-5180.

In some embodiments, the Fc region comprises modification corresponding to the combination of substitutions M428L/N434S as described in Zalevsky et al. Nat Biotechnol. (2010) 28:157-159.

In some embodiments, the Fc region comprises modification corresponding to the combination of substitutions S267E/L328F as described in Chu et al., Mol Immunol. (2008) 45:3926-3933. In some embodiments, the Fc region comprises modification corresponding to the combination of substitutions N325S/L328F as described in Shang et al. Biol Chem. (2014) 289:15309-15318.

In some embodiments, the Fc region comprises modification to reduce/prevent an Fc-mediated function. In some embodiments, the Fc region comprises modification to reduce/prevent ADCC. In some embodiments, the Fc region comprises modification to reduce/prevent ADCP. In some embodiments, the Fc region comprises modification to reduce/prevent CDC. An antigen-binding molecule comprising an Fc region comprising modification to reduce/prevent an Fc-mediated function (e.g. ADCC, ADCP, CDC) induces a reduced level of the relevant effector function as compared to an antigen-binding molecule comprising the corresponding unmodified Fc region. In some embodiments, the Fc region comprises modification to reduce/prevent binding to an Fc receptor. In some embodiments, the Fc region comprises modification to reduce/prevent binding to an Fcγ receptor. In some embodiments, the Fc region comprises modification to reduce/prevent binding to one or more of FcγRI, FcγRIIa, FcγRIIb, FcγRIIc, FcγRIIIa and FcγRIIIb. In some embodiments, the Fc region comprises modification to reduce/prevent binding to FcγRIIIa. In some embodiments, the Fc region comprises modification to reduce/prevent binding to FcγRIIa. In some embodiments, the Fc region comprises modification to reduce/prevent binding to FcγRIIb. In some embodiments, the Fc region comprises modification to reduce/prevent binding to a complement protein. In some embodiments, the Fc region comprises modification to reduce/prevent binding to C1q. In some embodiments, the Fc region comprises modification to reduce/prevent glycosylation of the amino acid residue corresponding to N297.

In some embodiments, the Fc region is not able to induce one or more Fc-mediated functions (i.e. lacks the ability to elicit the relevant Fc-mediated function(s)). Accordingly, antigen-binding molecules comprising such Fc regions also lack the ability to induce the relevant function(s). Such antigen-binding molecules may be described as being devoid of the relevant function(s). In some embodiments, the Fc region is not able to induce ADCC. In some embodiments, the Fc region is not able to induce ADCP. In some embodiments, the Fc region is not able to induce CDC. In some embodiments, the Fc region is not able to induce ADCC and/or is not able to induce ADCP and/or is not able to induce CDC. In some embodiments, the Fc region is not able to bind to an Fc receptor. In some embodiments, the Fc region is not able to bind to an Fcγ receptor. In some embodiments, the Fc region is not able to bind to one or more of FcγRI, FcγRIIa, FcγRIIb, FcγRIIc, FcγRIIIa and FcγRIIIb. In some embodiments, the Fc region is not able to bind to FcγRIIIa. In some embodiments, the Fc region is not able to bind to FcγRIIa. In some embodiments, the Fc region is not able to bind to FcγRIIb. In some embodiments, the Fc region is not able to bind to FcRn. In some embodiments, the Fc region is not able to bind to a complement protein. In some embodiments, the Fc region is not able to bind to C1q.

In some embodiments, the Fc region comprises modification corresponding to N297A or N297Q or N297G as described in Leabman et al., Mabs. (2013) 5:896-903. In some embodiments, the Fc region comprises modification corresponding to L235E as described in Alegre et al., J Immunol. (1992) 148:3461-3468. In some embodiments, the Fc region comprises modification corresponding to the combination of substitutions L234A/L235A or F234A/L235A as described in Xu et al., Cell Immunol. (2000) 200:16-26. In some embodiments, the Fc region comprises modification corresponding to P329A or P329G as described in Schlothauer et al., Protein Engineering, Design and Selection (2016), 29(10):457-466. In some embodiments, the Fc region comprises modification corresponding to the combination of substitutions L234A/L235A/P329G as described in Lo et al. J. Biol. Chem (2017) 292(9):3900-3908. In some embodiments, the Fc region comprises modification corresponding to the combination of substitutions described in Rother et al., Nat Biotechnol. (2007) 25:1256-1264. In some embodiments, the Fc region comprises modification corresponding to the combination of substitutions S228P/L235E as described in Newman et al., Clin. Immunol. (2001) 98:164-174. In some embodiments, the Fc region comprises modification corresponding to the combination of substitutions H268Q/V309L/A330S/P331S as described in An et al., Mabs. (2009) 1:572-579. In some embodiments, the Fc region comprises modification corresponding to the combination of substitutions V234A/G237A/P238S/H268A/V309L/A330S/P331S as described in Vafa et al., Methods. (2014) 65:114-126. In some embodiments, the Fc region comprises modification corresponding to the combination of substitutions L234A/L235E/G237A/A330S/P331S as described in US 2015/0044231 A1.

The combination of substitutions ‘L234A/L235A’ and corresponding substitutions (such as e.g. F234A/L235A in human IgG4) are known to disrupt binding of Fc to Fcγ receptors and inhibit ADCC, ADCP, and also to reduce C1q binding and thus CDC (Schlothauer et al., Protein Engineering, Design and Selection (2016), 29(10):457-466, hereby incorporated by reference in entirety). The substitutions ‘P329G’ and ‘P329A’ reduce C1q binding (and thereby CDC). Substitution of ‘N297’ with ‘A’, ‘G’ or ‘Q’ is known to eliminate glycosylation, and thereby reduce Fc binding to C1q and Fcγ receptors, and thus CDC and ADCC. Lo et al. J. Biol. Chem (2017) 292(9):3900-3908 (hereby incorporated by reference in its entirety) reports that the combination of substitutions L234A/L235A/P329G eliminated complement binding and fixation as well as Fcγ receptor dependent, antibody-dependent, cell-mediated cytotoxicity in both murine IgG2a and human IgG1.

The combination of substitutions L234A/L235E/G237A/A330S/P331S in IgG1 Fc is disclosed in US 2015/0044231 A1 to abolish induction of phagocytosis, ADCC and CDC.

In some embodiments, the Fc region comprises modification corresponding to the substitution S228P as described in Silva et al., J Biol Chem. (2015) 290(9):5462-5469. The substitution S228P in IgG4 Fc reduces Fab-arm exchange (Fab arm exchange can be undesirable).

In some embodiments, the Fc domain comprises a CH2-CH3 region comprising an amino acid difference at one or more of the following positions, relative to the amino acid sequence of a CH2-CH3 region of a reference Fc domain: 234, 235, 252, 254 or 256 (according to the EU numbering system). In some embodiments, the Fc domain comprises a CH2-CH3 region comprising amino acid differences at positions 234 and 235, relative to the amino acid sequence of a CH2-CH3 region of a reference Fc domain. In some embodiments, the Fc domain comprises a CH2-CH3 region comprising amino acid differences at positions 252, 254 and 256, relative to the amino acid sequence of a CH2-CH3 region of a reference Fc domain. In some embodiments, the Fc domain comprises a CH2-CH3 region comprising amino acid differences at positions 234, 235, 252, 254 and 256, relative to the amino acid sequence of a CH2-CH3 region of a reference Fc domain.

In some embodiments, the Fc domain comprises a CH2-CH3 region comprising one or more of the following specified amino acid residues: A234, A235, Y252, T254 or E256 (according to the EU numbering system).

In some embodiments, the Fc domain comprises a CH2-CH3 region comprising Y252, T254 and E256. These so called ‘YTE’ modifications located at the CH2-CH3 interface of the Fc domain have been shown to increase the binding affinity at pH 6.0 to the MHC Class I neonatal Fc receptor (FcRn), localised within the acidic endosomes of endothelial and haematopoietic cells, which increases efficient recycling of administered mAb and longer half-life in the plasma.

Previous studies have shown that Fcγ receptor interactions lead to uptake of ADCs by normal (i.e. non-cancerous) cells, and can lead to severe cytopenia (e.g. leukopenia)—see Zhao et al., Mol Cancer Ther (2017) 16(9): 1866-1876.

In some embodiments, the Fc domain comprises a CH2-CH3 region comprising A234 and A235. These so called ‘LALA’ modifications are known to suppress the interaction of the Fc-domain with Fcγ receptors expressed by hematopoietic cells, and potentially reduce Fc-Fcγ receptor interaction-mediated toxicities.

In some embodiments, the Fc region of the HER3-binding moiety according to the present disclosure comprises a CH2 domain comprising A234 and A235 (EU numbering). In some embodiments, the Fc region of the HER3-binding moiety comprises a CH2 domain comprising Y252, T254 and E256 (EU numbering).

In some embodiments, the Fc region of the HER3-binding moiety comprises a CH2 domain comprising A234, A235, Y252, T254 and E256 (EU numbering).

In some embodiments, the Fc domain comprises a CH2-CH3 region comprising A234, A235, Y252, T254 and E256.

In some embodiments, the Fc domain comprises a CH2-CH3 region comprising one or more of the following amino acid substitutions, relative to the amino acid sequence of a CH2-CH3 region of the reference Fc domain: L234A, L235A, M252Y, S254T or T256E (according to the EU numbering system). In some embodiments, the Fc domain comprises a CH2-CH3 region comprising L234A and L235A, relative to the amino acid sequence of a CH2-CH3 region of the reference Fc domain. In some embodiments, the Fc domain comprises a CH2-CH3 region comprising M252Y, S254T and T256E, relative to the amino acid sequence of a CH2-CH3 region of the reference Fc domain. In some embodiments, the Fc domain comprises a CH2-CH3 region comprising L234A, L235A, M252Y, S254T and T256E relative to the amino acid sequence of a CH2-CH3 region of the reference Fc domain.

In some embodiments, an Fc region according to the present disclosure comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or ≥100% sequence identity, to the amino acid sequence of SEQ ID NO:88, 89 or 90.

In some embodiments, an antigen-binding moiety according to the present disclosure comprises an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or ≥100% sequence identity, to the amino acid sequence of SEQ ID NO:94, 95 or 96.

An antigen-binding molecule according to the present disclosure may comprise a HER3-binding moiety according to any embodiment described hereinabove, and a linker-payload moiety comprising exatecan or a derivative thereof, as described hereinbelow. In preferred embodiments, the HER3-binding moiety is selected from one of (1) to (33) above.

### Linker-Payload Moiety

The antigen-binding molecules of the present disclosure comprise linker-payload moiety. As used herein, a linker-payload moiety refers to a moiety comprising a payload moiety, and a linker moiety for linking the payload moiety to the antigen-binding moiety (in the case of the present disclosure, the HER3-binding moiety).

The antigen-binding molecules of the present disclosure comprise a HER3-binding moiety and a linker-payload moiety comprising exatecan or a derivative thereof. That is, the payload moiety of the antigen-binding molecule of the present disclosure is exatecan or a derivative thereof.

Exatecan (also known as (1S,9S)-1-Amino-9-ethyl-5-fluoro-9-hydroxy-4-methyl-1,2,3,9,12,15-hexahydro-10H,13H-benzo[de]pyrano[3′,4′: 6,7]indolizino[1,2-b]quinoline-10,13-dione; DX-8951; DrugBank Acc. No. DB12185) is a DNA topoisomerase I inhibitor derivative of camptothecin, and has the structure:

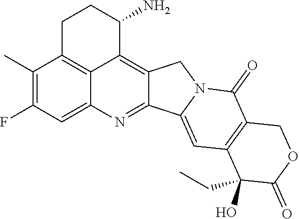 [SMILES: CC[C@@]1(O)C(=O)OCc2c1cc1n(c2=O)Cc2c-1nc1cc(F)c(C)c3c1c2[C@@H](N)CC3]

Exatecan is described e.g. in Takiguchi et al., Jpn J Cancer Res. (1997) 88(8):760-769, which is hereby incorporated by reference in its entirety. Exatecan and exatecan derivates have been investigated as a payload for antibody-drug conjugates—see e.g. Nakada et al., Bioorg. Med. Chem. Lett. (2016) 26:1542-1545, which is hereby incorporated by reference in its entirety.

The linker-payload moiety of the present disclosure comprises exatecan or a derivative thereof. In some embodiments, the linker-payload moiety comprises exatecan or an exatecan derivative described in WO 2022/058395 A1, which is hereby incorporated by reference in its entirety.

In some embodiments, the linker-payload moiety comprises exatecan, N-glycyl-exatecan or deruxtecan. In some embodiments, the antigen-binding molecule or linker-payload moiety of the present disclosure does not comprise deruxtecan. In some embodiments, the linker-payload moiety comprises exatecan or N-glycyl-exatecan. In some embodiments, the linker-payload moiety comprises exatecan.

The linker-payload moiety comprises a linker moiety. As used herein, a linker moiety refers to a moiety that connects two or more elements of a compound. Linker moieties for conjugating payload moieties to antigen-binding moieties are described e.g. in Su et al., Acta Pharmaceutica Sinica B (2021) 11(12):3889-3907 and Tsuchikama and An, Protein Cell. (2018) 9(1):33-46, both of which are hereby incorporated by reference in their entirety.

In some embodiments, the linker-payload moiety comprises a cleavable linker moiety. In some embodiments, the linker-payload moiety or linker moiety comprises a lysosomal protease-cleavable dipeptide. In some embodiments, the linker-payload moiety or linker moiety comprises a cathepsin (e.g. cathepsin B, K and/or L)-cleavable moiety. In some embodiments, the linker-payload moiety or linker moiety comprises a cathepsin B-cleavable moiety.

In some embodiments, the linker-payload moiety or linker moiety comprises the dipeptide valine-alanine or valine-citrulline. In some embodiments, the linker-payload moiety or linker moiety comprises the dipeptide valine-alanine.

In some embodiments, the linker-payload moiety or linker moiety comprises a self-immolative moiety. In some embodiments, the linker-payload moiety or linker moiety comprises a p-aminobenzyl carbamate (PABC) group.

In some embodiments, the linker-payload moiety or linker moiety comprises structure (D):

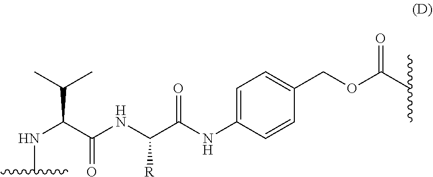

wherein R is selected from CH₃ and (CH₂)₃NHCOCNH₂.

In some embodiments, the linker-payload moiety comprises spacer moiety. As used herein, a spacer moiety refers to a moiety that spaces (i.e. provides distance between) and covalently links together two (or more) parts of a linker moiety. Spacer moieties include moieties comprising or consisting of: a polyethylene glycol (PEG) moiety, a polar acyl sulfamide moiety, a polar carbamoyl sulfamide moiety and/or a HydraSpace moiety.

In some embodiments, the linker-payload moiety or spacer moiety comprises a polyethylene glycol (PEG) moiety. In some embodiments, the linker-payload or spacer moiety comprises structure (G):

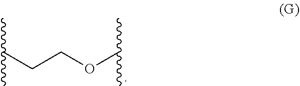 [SMILES: *CCO*]

In some embodiments, the linker-payload moiety or spacer moiety comprises a polar acyl sulfamide moiety or a polar carbamoyl sulfamide moiety. In some embodiments, the linker-payload moiety or spacer moiety comprises one or more HydraSpace moieties. As used herein, a HydraSpace moiety refers to a moiety having the structure (H):

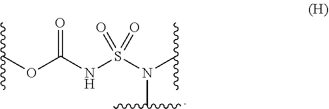 [SMILES: *OC(=O)NS(=O)(=O)N(*)*]

HydraSpace moieties are described e.g. in Verkade et al., Antibodies (Basel) (2018) 7(1):12 and WO 2016/053107 A1, both of which are hereby incorporated by reference in their entirety. In some embodiments, the linker-payload moiety comprises a structure of a compound described in WO 2016/053107 A1.

In some embodiments, the linker-payload moiety or spacer moiety comprises structure I:

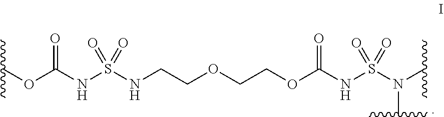 [SMILES: *OC(=O)NS(=O)(=O)NCCOCCOC(=O)NS(=O)(=O)N(*)*]

In preferred embodiments, the linker-payload moiety of the antigen-binding molecule of the present disclosure is a linker-payload described in WO 2022/058395 A1, which is incorporated by reference in its entirety.

In some embodiments, the antigen-binding linker-payload moiety comprises or consists of structure (I):

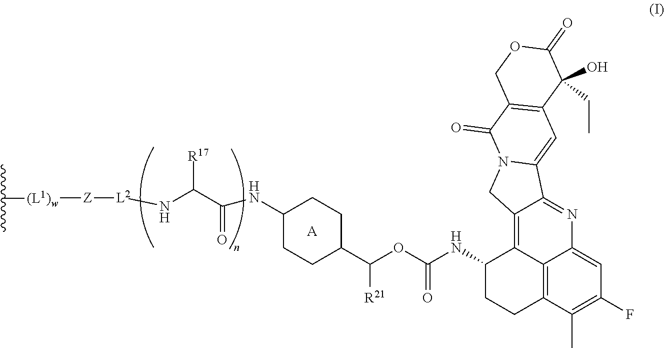

wherein:
L¹ and L² are linker moieties;
w is 0 or 1;
Z is a connecting group obtained by a metal-free click reaction or by thiol ligation;
each R¹⁷ is individually an amino acid side chain;
n is an integer in the range of 1-5;
A is a 5- or 6-membered aromatic or heteroaromatic ring;
x is an integer in the range of 1-8; and R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR²³ wherein R²³ is independently selected from the group consisting of hydrogen and C₁-C₄ alkyl groups.

In some embodiments, the linker-payload moiety comprises structure (J):

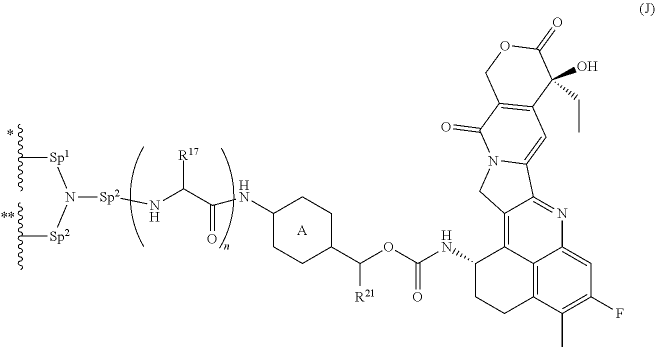

wherein:
the wavy bond labeled with * is connected to Z, and the wavy bond labeled with ** is connected to NH;
Sp¹ and Sp² are each individually spacer moieties; and
n, A R¹⁷ and R²¹ are as defined in structure (1) above.

That is, in some embodiments, L² of structure (1) comprises a branching moiety, preferably via a nitrogen atom, such that two payload moieties are connected to a single moiety Z. A ‘branching moiety’ refers to a moiety that is embedded in a linker connecting three moieties. In other words, the branching moiety comprises at least three bonds to other moieties, typically one bond to the HER-3-binding moiety, connecting to Z, one bond to the payload moiety and one bond to a second payload moiety. The branching moiety is preferably embedded in linker L².

In some embodiments, each occurrence of Sp² is the same. In some embodiments, each occurrence of (NH—CR¹⁷—CO)ₙ is the same. In some embodiments, each occurrence of A is the same. In some embodiments, each occurrence of R²¹ is the same.

In some embodiments, L² of structure (1) has structure (K):

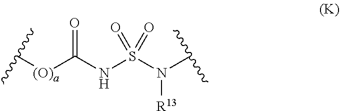

The wavy lines represent the connection to the remainder of the compound, typically to Z and to (NH—CR¹⁷—CO)ₙ, optionally via a spacer. Preferably, the (O)ₐC(O) moiety is connected to Z and the NR¹³ moiety to (NH—CR¹⁷—CO)ₙ. In case linker L² comprises a spacer moiety Sp¹, it is preferred that the sulfamide group according to structure (K) is comprised in spacer moiety Sp¹.

Therefore in some embodiments L² comprises structure (K), wherein:
a=0 or 1, and
R¹³ is selected from the group consisting of C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of C(O)X connected to N via a spacer moiety.

In some embodiments, L² comprises two groups of structure (K).

In some embodiments, linker L², preferably spacer Sp¹, comprises structure (L):

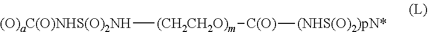

wherein: a is 0 or 1 and m is an integer in the range of 1-10, and p is 0 or 1. N* may represent a branching moiety.

In some embodiments, each occurrence of (NH—CR¹⁷—CO)ₙ is selected from Val-Cit, Val-Ala, Val-Lys, Val-Arg, AcLys-Val-Cit, AcLys-Val-Ala, Glu-Val-Ala, Asp-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn and Lys. In some embodiments, each occurrence of (NH—CR¹⁷—CO)ₙ is selected from Val-Cit, Val-Ala, Glu-Val-Ala, Val-Lys, Phe-Cit, Phe-Ala, Phe-Lys, Ala-Ala-Asn.

In some embodiments, each occurrence of (NH—CR¹⁷—CO)ₙ is Val-Cit or Val-Ala.

In some embodiments, Z of structure (I) has a structure selected from structure (Z1) to (Z21):

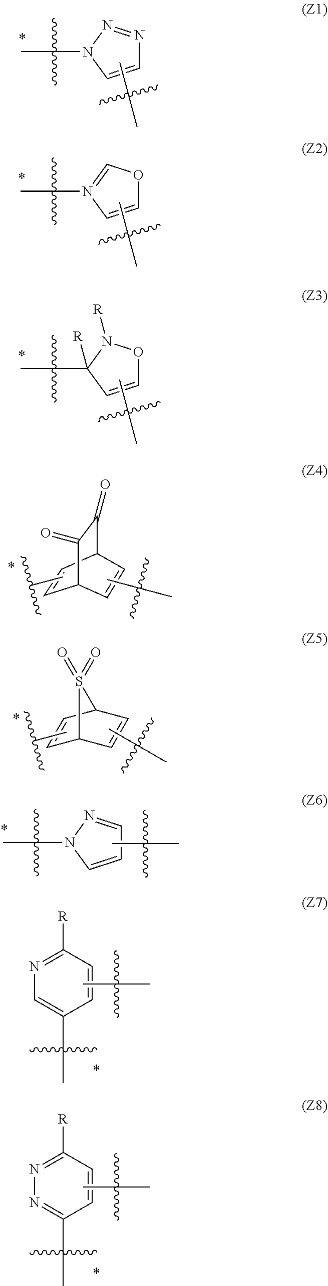

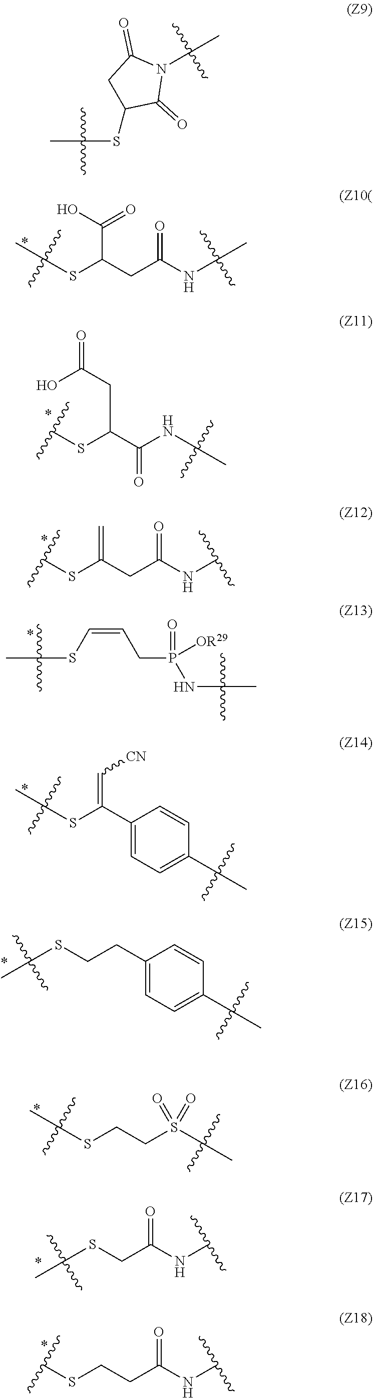

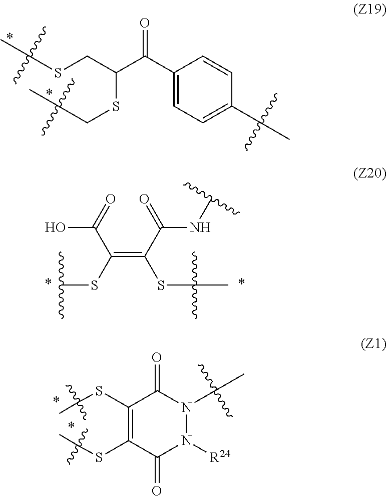

wherein:
the wavy bond labelled with * is connected to the HER3-binding moiety, optionally via L¹, and the other wavy bond is connected to L²;
functional groups R in (Z3), (Z7) and (Z8) are selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which may optionally be substituted and may optionally be interrupted by one or more heteroatoms selected from O, S and NR³², wherein R³² is independently selected from the group consisting of hydrogen and C₁-C₄ alkyl groups;
R²⁴ is H or C₁₋₁₂ alkyl, preferably H or C₁₋₆ alkyl;
R²⁹ is C₁₋₁₂ alkyl, preferably C₁₋₄ alkyl, most preferably ethyl.

In some embodiments, Z of structure (1) is according to structure (Z22):

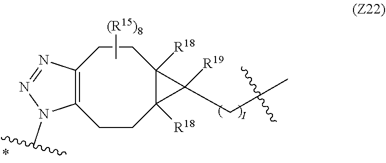

wherein:
the wavy bond labelled with * is connected to the HER3-binding moiety, optionally via L¹, and the other wavy bond is connected to L²;
R¹⁵ is independently selected from the group consisting of hydrogen, halogen, −OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H;
R¹⁸ is independently selected from the group consisting of hydrogen, C₁-C₆ alkyl groups, most preferably both R¹⁸ are H;
R¹⁹ is H;
I is 0 or 1, more preferably I is 1.

### Particular Exemplary Embodiments

In some embodiments, the antigen-binding molecule of the present disclosure comprises structure (L):

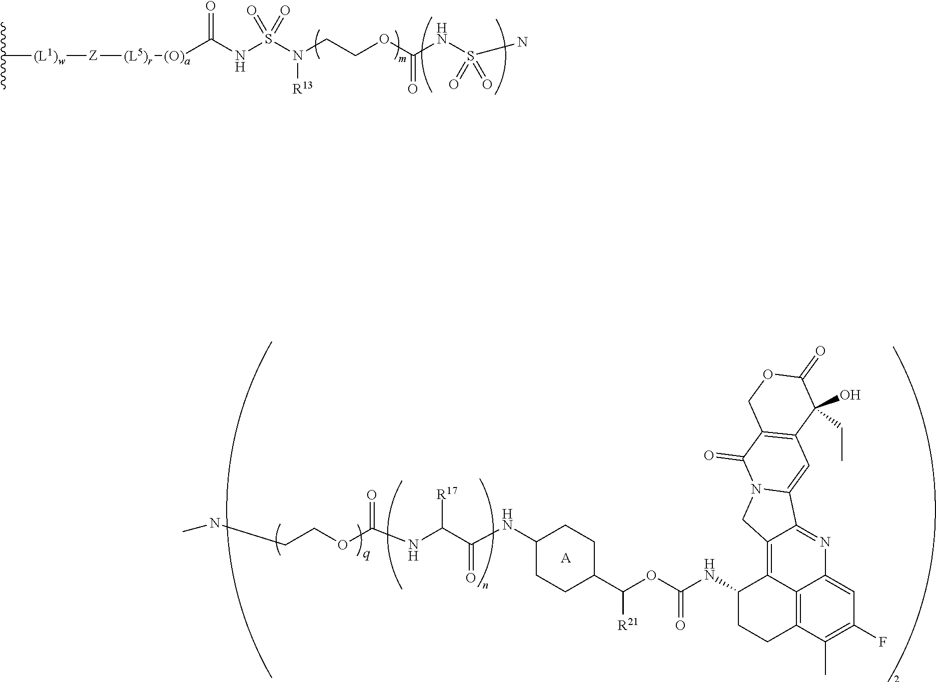

wherein:
L¹, Z, A, R²¹, n, R¹⁷ and x are as defined in structure (1) above;
a and R¹³ are as defined in structure (K) above;
L⁵ is a linker moiety;
r is 0 or 1;
m is an integer in the range of 1-10;
q is an integer in the range of 0-10; and
p is 0 or 1.

In some embodiments, the antigen-binding molecule of the present disclosure comprises structure (M):

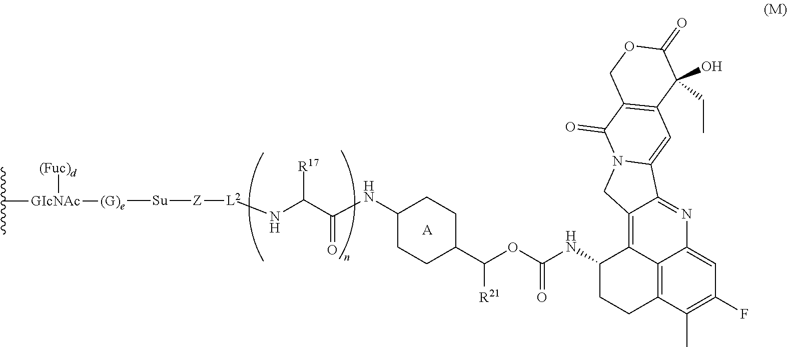

wherein Z, L², R¹⁷, A, R²¹, n and x are as defined in structure (1) above;
e is an integer in the range of 0-20;
Su is a monosaccharide;
G is a monosaccharide moiety;
GlcNAc is an N-acetylglucosamine moiety;
Fuc is a fucose moiety; and
d is 0 or 1.

In some embodiments, the antigen-binding molecule of the present disclosure comprises structure (N):

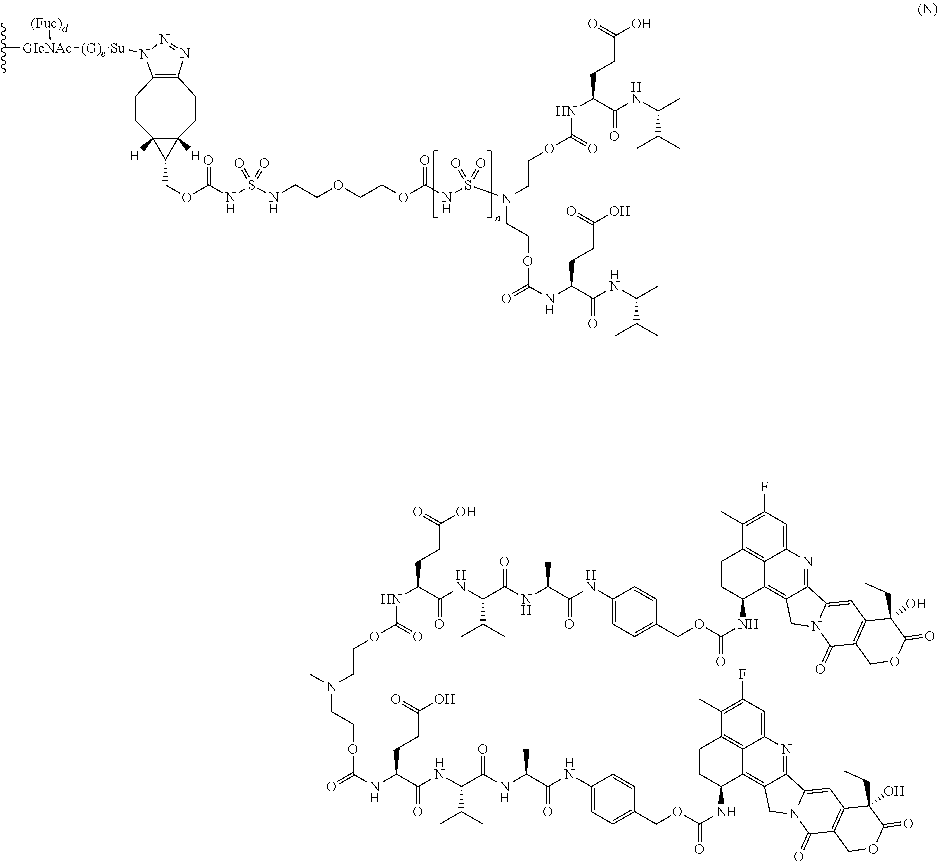

wherein:
e, Su, G, GlcNAc, Fuc and d are as defined in structure (M) above; and
n is 0 or 1.

In some embodiments, the antigen-binding molecule of the present disclosure comprises SYNtecan E. That is, in some embodiments, the antigen-binding molecules of the present disclosure comprise a HER3-binding moiety and SYNtecan E.

As used herein, ‘SYNtecan E’ refers to a moiety consisting of the following structure:

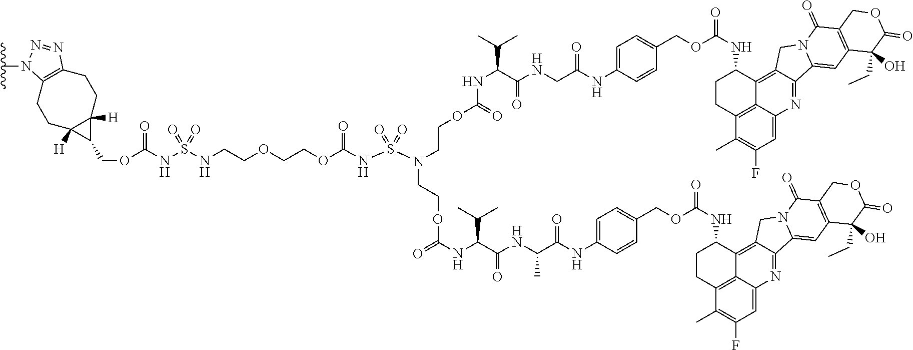 [SMILES: *n1nnc2c1CC[C@@H]1[C@H](CC2)[C@@H]1COC(=O)NS(=O)(=O)NCCOCCOC(=O)NS(=O)(=O)N(CCOC(=O)N[C@H](C(=O)NCC(=O)Nc1ccc(COC(=O)N[C@H]2CCc3c(C)c(F)cc4nc5c(c2c34)Cn2c-5cc3c(c2=O)COC(=O)[C@]3(O)CC)cc1)C(C)C)CCOC(=O)N[C@H](C(=O)N[C@@H](C)C(=O)Nc1ccc(COC(=O)N[C@H]2CCc3c(C)c(F)cc4nc5c(c2c34)Cn2c-5cc3c(c2=O)COC(=O)[C@]3(O)CC)cc1)C(C)C]

### Functional Properties of the Antigen-Binding Molecules

The antigen-binding molecules described herein may be characterised by reference to certain functional properties. In some embodiments, an antigen-binding molecule described herein may possess one or more of the following properties:
binds to cells expressing HER3;
binds to cells expressing HER3 in the presence of NRG1;
inhibits proliferation of HER3-expressing cells;
increases killing of cells expressing HER3;
inhibits tumor growth and/or reduces tumor size/volume (e.g. of a HER3-expressing cancer); increases survival of subjects having a cancer (e.g. a HER3-expressing cancer);
does not display substantial Fc-mediated binding to PBMCs (e.g. human PBMCs);
does not display substantial binding to human Fcγ receptors (e.g. FcγRIA, FcγRIIA and/or FcγRIIIA);
is internalized into cells expressing HER3;
does not display substantial macropinocytosis-dependent uptake by human megakaryocytes;
does not induce substantial leukopenia;
does not induce substantial neutropenia;
does not induce substantial thrombocytopenia;
does not induce substantial anemia;
does not induce substantial interstitial lung disease;
does not substantially impair hepatic function;
does not substantially impair renal function; and/or does not substantially impair pulmonary function.

It will be appreciated that a given antigen-binding molecule may display more than one of the properties recited in the preceding paragraph. A given antigen-binding molecule may be evaluated for the properties recited in the preceding paragraph using suitable assays. For example, the assays may be e.g. in vitro assays, optionally cell-based assays or cell-free assays. In some embodiments, the assays may be e.g. in vivo assays, i.e. performed in non-human animals. In some embodiments, the assays may be e.g. ex vivo assays, i.e. performed using cells/tissue/an organ obtained from a subject.

Where assays are cell-based assays, they may comprise treating cells with an antigen-binding molecule in order to determine whether the antigen-binding molecule displays one or more of the recited properties. Assays may employ species labelled with detectable entities in order to facilitate their detection. Assays may comprise evaluating the recited properties following treatment of cells separately with a range of quantities/concentrations of a given antigen-binding molecule (e.g. a dilution series).

Analysis of the results of such assays may comprise determining the concentration at which 50% of the maximal level of the relevant activity is attained. The concentration of a given agent at which 50% of the maximal level of the relevant activity is attained may be referred to as the ‘half-maximal effective concentration’ of the agent in relation to the relevant activity, which may also be referred to as the ‘EC₅₀’. Depending on the property, the EC₅₀ may also be referred to as the ‘half-maximal inhibitory concentration’ or ‘IC₅₀’, this being the concentration of the agent at which 50% of the maximal level of inhibition of a given property is observed.

In some embodiments, the antigen-binding molecule of the present disclosure binds to HER3 in a region which is accessible to an antigen-binding molecule (i.e., an extracellular antigen-binding molecule) when HER3 is expressed at the cell surface (i.e. in or at the cell membrane). In some embodiments, the antigen-binding molecule binds to HER3 expressed at the cell surface of a cell expressing HER3. In some embodiments, the antigen-binding molecule binds to HER3-expressing cells (e.g. H358, T47D or OVCAR8, HCT116 or DU145 cells).

The ability of an antigen-binding molecule to bind to a given cell type can be analysed by contacting cells with the antigen-binding molecule, and detecting antigen-binding molecule bound to the cells, e.g. after a washing step to remove unbound antigen-binding molecule. The ability of an antigen-binding molecule to bind to HER3-expressing cells can be analysed by methods such as flow cytometry and immunofluorescence microscopy. For example, the ability of an antigen-binding molecule to bind to HER3-expressing cells can be evaluated as described in Example 3.1 herein.

In some embodiments, the antigen-binding molecule described herein binds to cells expressing human HER3 with an EC₅₀ of 10 nM or less, preferably one of ≤5 nM, ≤4 nM, ≤3 nM, ≤2 nM, ≤1 nM, ≤900 pM, ≤800 pM, ≤700 pM, ×600 pM or ≤500 pM, e.g. as determined by analysis as described in Example 3.1.

In some embodiments, the antigen-binding molecule is capable of binding to HER3 and/or HER3-expressing cells in the presence and/or absence of a ligand for HER3 (e.g. NRG, NRG1 and/or NRG2). In some embodiments, the antigen-binding molecule is capable of binding to HER3 and/or HER3-expressing cells independently of a ligand for HER3 (e.g. NRG, NRG1 and/or NRG2). In some embodiments, the antigen-binding molecule does not compete with a ligand for HER3 (e.g. NRG, NRG1 and/or NRG2) for binding to HER3 and/or HER3-expressing cells. In some embodiments, the antigen-binding molecule does not bind to HER3 at the ligand binding site. The ability of an antigen-binding molecule to bind to HER3-expressing cells can be evaluated as described in Example 3.2 herein.

In some embodiments, the antigen-binding molecule inhibits proliferation of HER3-expressing cells (e.g. HER3-expressing cancer cells). The ability of an antigen-binding molecule to inhibit proliferation of a given cell type can be analysed by contacting cells with the antigen-binding molecule in the presence of a ligand for HER3 (e.g. NRG1), and subsequently evaluating proliferation of the cells (i.e. after a period of time sufficient for an effect on cell proliferation to be observed). Cell proliferation can be evaluated e.g. by detecting changes in number of cells over time, or by in vitro analysis of incorporation of ³H-thymidine or by CFSE dilution assay, e.g. as described in Fulcher and Wong, Immunol Cell Biol (1999) 77(6): 559-564, hereby incorporated by reference in entirety. For example, the ability of an antigen-binding molecule to inhibit proliferation of HER3-expressing cells can be evaluated as described in Example 3.4 herein. In this example, Cell counting kit—8 (CCK8) was used to measure cell proliferation. CCK8 contains a tetrazolium salt that is reduced by cellular dehydrogenases to an orange formazan product, which is soluble in tissue culture medium. The amount of formazan produced is directly proportional to the number of living cells, and is measured by absorbance at 460 nm (Li et al., Cell Prolif. (2021) 54(3):e12986; Zhang et al., Genome Biol. (2021) 22(1):41).

In some embodiments, the antigen-binding molecule of the present invention is capable of inhibiting proliferation of HER3-expressing cells to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 0.01 times the level of proliferation of HER3-expressing cells observed in the absence of the antigen-binding molecule (or in the presence of an appropriate control antigen-binding molecule known not to influence proliferation of HER3-expressing cells), in a given assay.

In some embodiments, the antigen-binding molecule described herein inhibits proliferation of cells expressing human HER3 with an IC₅₀ of 100 nM or less, preferably one of ≤50 nM, ≤40 nM, ≤30 nM, ≤20 nM, ≤10 nM, ≤5 nM, ≤4 nM, ≤3 nM, ≤2 nM, ≤1 nM, ≤900 pM, ≤800 pM, ≤700 pM, ≤600 pM or ≤500 pM, e.g. as determined by analysis as described in Example 3.4.

In some embodiments, the antigen-binding molecule according to the present disclosure potentiates (i.e. upregulates, enhances) cell killing of cells comprising/expressing HER3.

In some embodiments, an antigen-binding molecule according to the present disclosure may inhibit growth or reduce metastasis of a cancer comprising cells comprising/expressing HER3. In some embodiments, an antigen-binding molecule may potentiate (i.e. upregulate, enhance) cell killing of cells comprising/expressing HER3. In some embodiments, an antigen-binding molecule may inhibit growth of cells of a cancer, or may inhibit growth of a tumor, comprising cells comprising/expressing HER3. In some embodiments, an antigen-binding molecule may inhibit metastasis of a cancer/tumor comprising cells comprising/expressing HER3.

Cell killing can be investigated, for example, using any of the methods reviewed in Zaritskaya et al., Expert Rev Vaccines (2011), 9(6):601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells.

In some embodiments an antigen-binding molecule according to the present disclosure is capable of reducing the number/proportion of cells expressing HER3. In some embodiments, an antigen-binding molecule according to the present disclosure is capable of depleting/enhancing depletion of such cells.

In some embodiments, an antigen-binding molecule of the present disclosure displays anticancer activity. In some embodiments, the antigen-binding molecule increases killing of cancer cells. In some embodiments, the antigen-binding molecule causes a reduction in the number of cancer cells in vivo, e.g. as compared to an appropriate control condition. The cancer may be a cancer as described herein, e.g. a cancer expressing/overexpressing HER3.

In some embodiments, an antigen-binding molecule according to the present disclosure reduces/inhibits growth of a cancer and/or of a tumor of a cancer. In some embodiments, an antigen-binding molecule reduces tissue invasion by cells of a cancer. In some embodiments, an antigen-binding molecule reduces metastasis of a cancer. In some embodiments, an antigen-binding molecule displays anticancer activity. In some embodiments, an antigen-binding molecule reduces the growth/proliferation of cancer cells. In some embodiments, an antigen-binding molecule reduces the survival of cancer cells. In some embodiments, an antigen-binding molecule increases the killing of cancer cells. In some embodiments, an antigen-binding molecule of the present disclosure causes a reduction in the number of cancer cells e.g. in vivo. The cancer may be a cancer comprising cells expressing HER3.

An antigen-binding molecule of the present disclosure may be analysed for the properties described in the preceding paragraph in appropriate assays. Such assays include e.g. in vivo models. Antigen-binding molecules may be evaluated for such properties in experiments performed essentially as described in Example 4.1 herein.

In some embodiments, administration of an antigen-binding molecule according to the present disclosure may cause one or more of: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of one or more symptoms of the cancer, a reduction in the number of cancer cells, a reduction in the cancer burden, a reduction in tumour size/volume, and/or an increase in survival of subjects having the cancer (e.g. progression free survival or overall survival), e.g. as determined in an appropriate model.

It will be appreciated that the properties recited in the preceding paragraph are evaluated after a period of time sufficient for an effect associated with treatment using the antigen-binding molecule to be observed. Tumor growth may be monitored by investigating tumor volume over time. Tumor growth may be evaluated by measuring tumor volume (e.g. in mm³) over time.

In some embodiments, an antigen-binding molecule of the present disclosure is capable of reducing tumor size/volume (e.g. the mean tumor size/volume for the treatment group in an in vivo model, e.g. of a HER3-expressing cancer) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the tumor size/volume observed at the same time point in the absence of treatment with the antigen-binding molecule (or following treatment with an appropriate control antigen-binding molecule known not to influence tumor growth), in a given assay. In some embodiments, evaluation of tumor size/volume for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first dose of the antigen-binding molecule, in the relevant model.

In some embodiments, an antigen-binding molecule of the present disclosure achieves a level of tumor growth inhibition (e.g. expressed as % tumor growth inhibition, e.g. calculated relative to tumor growth observed on treatment with an appropriate control antigen-binding molecule) which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of tumor growth inhibition observed at the same time point in the absence of treatment with the antigen-binding molecule (or following treatment with an appropriate control antigen-binding molecule known not to influence tumor growth), in a given assay. In some embodiments, evaluation of tumor growth inhibition for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first dose of the antigen-binding molecule, in the relevant model.

In some embodiments, an antigen-binding molecule of the present disclosure is capable of increasing median survival of subjects having a cancer (e.g. in an in vivo model, e.g. of a HER3-expressing cancer) to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the median survival observed in the absence of treatment with the antigen-binding molecule (or following treatment with an appropriate control antigen-binding molecule known not to influence survival of subjects having the cancer), in a given assay. Median survival may be expressed in days from the start of the experiment, for subjects in the relevant treatment groups.

In some embodiments, the antigen-binding molecule of the present disclosure does not display substantial Fc-mediated binding to PBMCs, e.g. human PBMCs. In some embodiments, the antigen-binding molecule of the present disclosure does not display substantial binding to human Fcγ receptors (e.g. FcγRIA, FcγRIIA and/or FcγRIIIA). Such binding may be determined by, for example, surface plasmon resonance or fluorescence techniques as described herein.

In some embodiments, the antigen-binding molecule of the present disclosure is capable of being internalized into cells expressing HER3, for example into the lysosomes of said cells. Such internalization may be determined by, for example, fluorescence-based imaging techniques as described herein.

In some embodiments, the antigen-binding molecule of the present disclosure does not display substantial macropinocytosis-dependent uptake by megakaryocytes. Macropinocytosis-dependent uptake may be determined by, for example, fluorescence-based live cell imaging techniques as described herein.

In some embodiments, the antigen-binding molecule of the present disclosure does not induce substantial leukopenia (i.e. reduction in the number/proportion of white blood cells in the peripheral blood) following administration to a subject. In some embodiments, the antigen-binding molecule of the present disclosure does not induce substantial neutropenia (i.e. reduction in the number/proportion of neutrophils in the peripheral blood) following administration to a subject. In some embodiments, the antigen-binding molecule of the present disclosure does not induce substantial thrombocytopenia (i.e. reduction in the number/proportion of platelets in the peripheral blood) following administration to a subject. In some embodiments, the antigen-binding molecule of the present disclosure does not induce substantial anemia (i.e. reduction in the number/proportion of red blood cells in the peripheral blood) following administration to a subject.

An antigen-binding molecule can be evaluated in order to determine whether it induces leukopenia, neutropenia, thrombocytopenia and/or anemia by measuring the number/proportion of white blood cells/neutrophils/platelets/red blood cells in the peripheral blood following administration of the antigen-binding molecule to a subject. Such evaluation may comprise collecting a blood sample from the subject following administration of the antigen-binding molecule to the subject. It will be appreciated that evaluation is performed after a sufficient period of time for leukopenia/neutropenia/thrombocytopenia/anemia to be observed. Measurement of the number/proportion of the given cell type may be performed by any suitable means, e.g. by flow cytometry following antibody-based labelling of cellular biomarkers capable of distinguishing blood cells of different type.

An antigen-binding molecule that does not substantially induce leukopenia/neutropenia/thrombocytopenia/anemia may be an antigen-binding molecule for which no substantial reduction in the number/proportion of white blood cells/neutrophils/platelets/red blood cells in the peripheral blood is observed following administration of the antigen-binding molecule, as compared to the number/proportion of white blood cells/neutrophils/platelets/red blood cells in the peripheral blood of a subject not treated with the antigen-binding molecule, or in the peripheral blood of a subject treated with an appropriate control antigen-binding molecule known not to induce leukopenia/neutropenia/thrombocytopenia/anemia.

In some embodiments, the number/proportion of white blood cells/neutrophils/platelets/red blood cells in the peripheral blood of a subject following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the number/proportion of such cells observed in the peripheral blood of a subject that has not been administered the antigen-binding molecule, or in the peripheral blood of a subject that has been administered an appropriate control antigen-binding molecule known not to induce leukopenia/neutropenia/thrombocytopenia/anemia. In some embodiments, the number/proportion of white blood cells/neutrophils/platelets/red blood cells in the peripheral blood of a subject following administration of an antigen-binding molecule according to the present disclosure is ≥0.5 times, e.g. one of ≥0.55 times, ≥0.6 times, ≥0.65 times, ≥0.7 times, ≥0.75 times, ≥0.8 times, ≥0.85 times, ≥0.9 times or ≥0.95 times the number/proportion of such cells observed in the peripheral blood of a subject that has not been administered the antigen-binding molecule, or in the peripheral blood of a subject that has been administered an appropriate control antigen-binding molecule known not to induce leukopenia/neutropenia/thrombocytopenia/anemia.

In some embodiments, the antigen-binding molecule of the present disclosure does not induce substantial interstitial lung disease following administration to a subject.

An antigen-binding molecule can be evaluated in order to determine whether it induces interstitial lung disease by evaluating a subject for one or more correlates of interstitial lung disease (e.g. fibrosis of tissue of the lung, extracellular matrix deposition in tissue of the lung, expression of profibroinflammatory genes in tissue of the lung) following administration of the antigen-binding molecule to the subject. Such evaluation may comprise collecting a sample of lung tissue following administration of the antigen-binding molecule to the subject. It will be appreciated that evaluation is performed after a sufficient period of time for interstitial lung disease to have been induced by administration of the antigen-binding molecule.

An antigen-binding molecule that does not substantially induce interstitial lung disease may be an antigen-binding molecule for which no substantial increase in the level of a correlate of interstitial lung disease is observed following administration of the antigen-binding molecule, as compared to the level of the relevant correlate in a subject not treated with the antigen-binding molecule, or the level of the relevant correlate in a subject treated with an appropriate control antigen-binding molecule known not to induce interstitial lung disease.

In some embodiments, the level of a correlate of interstitial lung disease following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the level of the relevant correlate in a subject that has not been administered the antigen-binding molecule, or in a subject that has been treated with an appropriate control antigen-binding molecule known not to induce interstitial lung disease.

In some embodiments, the antigen-binding molecule of the present disclosure does not substantially impair hepatic function following administration to a subject. In some embodiments, the antigen-binding molecule does not display substantial hepatotoxicity. In some embodiments, the antigen-binding molecule does not cause substantial damage to liver cells (e.g. hepatocytes) and/or hepatic tissue. In some embodiments, the antigen-binding molecule of the present disclosure does not substantially impair renal function following administration to a subject. In some embodiments, the antigen-binding molecule does not display substantial nephrotoxicity. In some embodiments, the antigen-binding molecule does not cause substantial damage to kidney cells and/or renal tissue. In some embodiments, the antigen-binding molecule of the present disclosure does not substantially impair lung function following administration to a subject. In some embodiments, the antigen-binding molecule does not display substantial pulmonary toxicity. In some embodiments, the antigen-binding molecule does not cause substantial damage to lung cells and/or pulmonary tissue.

An antigen-binding molecule can be evaluated in order to determine whether it impairs hepatic/renal/lung function by measuring correlates of hepatic/renal/lung function following administration of the antigen-binding molecule to a subject. Such evaluation may comprise collecting a blood sample from the subject following administration of the antigen-binding molecule to the subject. It will be appreciated that evaluation is performed after a sufficient period of time for impairment of hepatic/renal/lung function to be observed. Impairment of hepatic function may be determined by detection of an increase in the level of a liver transaminase (such as aspartate transaminase (AST) and/or alanine transaminase (ALT)) in the peripheral blood, relative to the level observed in the peripheral blood of a subject not treated with the antigen-binding molecule, or in the peripheral blood of a subject treated with an appropriate control antigen-binding molecule known not to impair hepatic function. Impairment of renal function may be determined by detection of an increase in the level/concentration of blood urea nitrogen (BUN) and/or creatinine in the peripheral blood, relative to the level observed in the peripheral blood of a subject not treated with the antigen-binding molecule, or in the peripheral blood of a subject treated with an appropriate control antigen-binding molecule known not to impair renal function. Lung function may be evaluated e.g. by spirometry, plethysmography and/or by analysis of blood oxygen levels. Impairment of lung function may be determined by detection of a reduction in one or more of the following, relative to the level displayed by a subject not treated with the antigen-binding molecule, or a subject treated with an appropriate control antigen-binding molecule known not to impair lung function: blood oxygen level, tidal volume (TV), minute volume (MV), vital capacity (VC), functional residual capacity (FRC), total lung capacity, forced vital capacity (FVC), forced expiratory volume (FEV), forced expiratory flow (FEF) and/or peak expiratory flow rate (PEFR).

In some embodiments, the level of a correlate of impairment of hepatic function (e.g. AST and/or ALT) in the peripheral blood of a subject following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the level observed in the peripheral blood of a subject that has not been administered the antigen-binding molecule, or in the peripheral blood of a subject that has been administered an appropriate control antigen-binding molecule known not to impair hepatic function. In some embodiments, the level of a correlate of impairment of hepatic function (e.g. AST and/or ALT) in the peripheral blood of a subject following administration of an antigen-binding molecule according to the present disclosure is ≤2 times, e.g. one of ≤1.9 times, ≤1.8 times, ≤1.7 times, ≤1.6 times, ≤1.5 times, ≤1.4 times, ≤1.3 times, ≤1.2 times or ≤1.1 times the level observed in the peripheral blood of a subject that has not been administered the antigen-binding molecule, or in the peripheral blood of a subject that has been administered an appropriate control antigen-binding molecule known not to impair hepatic function.

In some embodiments, the level/concentration of a correlate of impairment of renal function (e.g. BUN and/or creatinine) in the peripheral blood of a subject following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the level/concentration observed in the peripheral blood of a subject that has not been administered the antigen-binding molecule, or in the peripheral blood of a subject that has been administered an appropriate control antigen-binding molecule known not to impair renal function. In some embodiments, the level/concentration of a correlate of impairment of renal function (e.g. BUN and/or creatinine) in the peripheral blood of a subject following administration of an antigen-binding molecule according to the present disclosure is ≤2 times, e.g. one of ≤1.9 times, ≤1.8 times, ≤1.7 times, ≤1.6 times, ≤1.5 times, ≤1.4 times, ≤1.3 times, ≤1.2 times or ≤1.1 times the level observed in the peripheral blood of a subject that has not been administered the antigen-binding molecule, or in the peripheral blood of a subject that has been administered an appropriate control antigen-binding molecule known not to impair renal function.

In some embodiments, the level of a correlate of lung function in a subject following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the level of the correlate observed in a subject that has not been administered the antigen-binding molecule, or in a subject that has been administered an appropriate control antigen-binding molecule known not to impair lung function.

In some embodiments, an antigen-binding molecule according to the present disclosure possesses one or more novel, similar or improved functional properties as compared to a known anti-HER3 antibody-drug conjugate. In some embodiments, an antigen-binding molecule possesses one or more novel, similar or improved functional properties as compared to Patritumab-DXd (U3-1402). Patritumab-DXd (also known as U3-1402; FDA UNII: 3XP17EG4W8) is described e.g. in Proposed INN: List 121, WHO Drug Information (2019) 33(2):314-316. It comprises the HER3-binding monoclonal antibody Patritumab, conjugated via a cleavable tetrapeptide maleimide-Gly-Gly-Phe-Gly linker to the DNA topoisomerase I inhibitor deruxtecan (DX-8951), with a drug-to-antibody ratio (DAR) of 8.

In some embodiments, an antigen-binding molecule described herein may display one or more of the following:
similar or increased binding to cells expressing HER3, as compared to binding to such cells displayed by Patritumab-DXd;
increased binding to cells expressing HER3 in the presence of NRG1, as compared to binding to such cells displayed by Patritumab-DXd;
similar or increased inhibition of proliferation of HER3-expressing cells, as compared to inhibition of proliferation of such cells displayed by Patritumab-DXd;
similar or increased killing of cells expressing HER3, as compared to killing of such cells displayed by Patritumab-DXd;
similar or increased inhibition of tumor growth and/or reduction of tumor size/volume (e.g. of a HER3-expressing cancer), as compared to tumor growth inhibition/tumor size/volume reduction displayed by Patritumab-DXd; similar or increased survival of subjects having a cancer (e.g. a HER3-expressing cancer) following treatment with the antigen-binding molecule, as compared to survival observed following treatment with Patritumab-DXd;
similar or decreased Fc-mediated binding to human PBMCs, as compared to Fc-mediated binding displayed by Patritumab-DXd;
similar or increased internalization into cells expressing HER3, as compared to internalization displayed by Patritumab-DXd;
similar or decreased macropinocytosis-dependent uptake by megakaryocytes, as compared to macropinocytosis-dependent uptake by Patritumab-DXd;
similar or reduced toxicity, as compared to Patritumab-DXd;
similar or reduced induction of leukopenia, as compared to Patritumab-DXd;
similar or reduced induction of neutropenia, as compared to Patritumab-DXd;
similar or reduced induction of anemia, as compared to Patritumab-DXd;
similar or reduced induction of thrombocytopenia, as compared to Patritumab-DXd;
similar or reduced induction of interstitial lung disease, as compared to Patritumab-DXd; similar or reduced impairment of hepatic function, as compared to Patritumab-DXd;
similar or reduced impairment of renal function, as compared to Patritumab-DXd; and/or similar or reduced impairment of pulmonary function, as compared to Patritumab-DXd.

In accordance with the preceding paragraph, a level of a given property/outcome which is ‘similar to’ a reference level may be ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the reference level. In some embodiments, a level of a given property/outcome which is ‘increased’ relative to a reference level may be greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times or ≥5 times the reference level.

It will be appreciated that for the purposes of such evaluations, equivalent amounts of the antigen-binding molecule and Patritumab-DXd may be compared. In some embodiments, equivalent amounts of the respective payloads are compared. That is, in some embodiments, for the purposes of such evaluations, amounts of the antigen-binding molecule and Patritumab-DXd are compared such that the amount of exatecan and deruxtecan is equivalent.

In some embodiments, the antigen-binding molecule of the present disclosure binds to HER3-expressing cells (e.g. in the presence of NRG1) with an EC₅₀ which is similar to or less than the EC₅₀ with which Patritumab-DXd binds to the relevant cells, in a given assay. In some embodiments, the EC₅₀ for binding of the antigen-binding molecule to HER3-expressing cells is ≥0.5 times and ≤2 times, e.g. one of 0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the EC₅₀ for binding of Patritumab-DXd to cells of the same type, as determined in the same assay. In some embodiments, the EC₅₀ for binding of the antigen-binding molecule to HER3-expressing cells is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 0.01 times the EC₅₀ for binding of Patritumab-DXd to cells of the same type, as determined in the same assay.

In some embodiments, the antigen-binding molecule of the present disclosure inhibits the proliferation of HER3-expressing cells (e.g. HER3-expressing cancer cells) with an IC₅₀ which is similar to or less than the IC₅₀ with which Patritumab-DXd inhibits proliferation of the relevant cells, in a given assay. In some embodiments, the antigen-binding molecule inhibits the proliferation of HER3-expressing cells (e.g. HER3-expressing cancer cells) with an IC₅₀ that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and 1.1 times the IC₅₀ for inhibition of proliferation of cells of the same type by Patritumab-DXd, as determined in the same assay. In some embodiments, the IC₅₀ for inhibition of the proliferation of HER3-expressing cells (e.g. HER3-expressing cancer cells) for the antigen-binding molecule is less than 1 times, e.g. 0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 0.01 times the IC₅₀ for inhibition of proliferation of cells of the same type by Patritumab-DXd, as determined in the same assay.

In some embodiments, the antigen-binding molecule of the present disclosure increases the killing of cells expressing HER3 to a level that is similar to or greater than the level of killing of the relevant cells displayed by Patritumab-DXd, in a given assay. In some embodiments, the antigen-binding molecule increases the killing of HER3-expressing cells (e.g. HER3-expressing cancer cells) to a level that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the level of cell killing of the relevant cells displayed by Patritumab-DXd, as determined in the same assay. In some embodiments, the antigen-binding molecule increases the killing of HER3-expressing cells (e.g. HER3-expressing cancer cells) to a level that is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of cell killing of the relevant cells displayed by Patritumab-DXd, as determined in the same assay.

In some embodiments, the antigen-binding molecule of the present disclosure reduces tumor size/volume to a level that is similar to or greater than the level of reduction in tumor size/volume observed following treatment with Patritumab-DXd, in a given assay. In some embodiments, the antigen-binding molecule is capable of reducing tumor size/volume (e.g. the mean tumor size/volume for the treatment group in an in vivo model, e.g. of a HER3-expressing cancer) to a level that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the tumor size/volume observed at the same time point following treatment with Patritumab-DXd, as determined in the same assay. In some embodiments, the antigen-binding molecule is capable of reducing tumor size/volume (e.g. the mean tumor size/volume for the treatment group in an in vivo model, e.g. of a HER3-expressing cancer) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 0.01 times the tumor size/volume observed at the same time point following treatment with Patritumab-DXd, as determined in the same assay. In some embodiments, evaluation of tumor size/volume for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or 100 days following administration of the first dose of the relevant antigen-binding molecule, in the relevant model.

In some embodiments, the antigen-binding molecule of the present disclosure achieves a level of tumor growth inhibition that is similar to or greater than the tumor growth inhibition observed following treatment with Patritumab-DXd, in a given assay. In some embodiments, the antigen-binding molecule achieves a level of tumor growth inhibition (e.g. expressed as % tumor growth inhibition, e.g. calculated relative to tumor growth observed on treatment with an appropriate control antigen-binding molecule) which is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the level of tumor growth inhibition observed at the same time point following treatment with Patritumab-DXd, as determined in the same assay. In some embodiments, the antigen-binding molecule achieves a level of tumor growth inhibition (e.g. expressed as % tumor growth inhibition, e.g. calculated relative to tumor growth observed on treatment with an appropriate control antigen-binding molecule) which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or 10 times the level of tumor growth inhibition observed at the same time point following treatment with Patritumab-DXd, as determined in the same assay. In some embodiments, evaluation of tumor size/volume for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or 100 days following administration of the first dose of the relevant antigen-binding molecule, in the relevant model.

In some embodiments, the antigen-binding molecule of the present disclosure achieves an increase in median survival of subjects having a cancer (e.g. in an in vivo model, e.g. of a HER3-expressing cancer) that is similar to or greater than the increase in median survival observed following treatment with Patritumab-DXd, in a given assay. In some embodiments, the antigen-binding molecule achieves an increase in median survival of subjects having a cancer (e.g. in an in vivo model, e.g. of a HER3-expressing cancer) that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the level of increase in median survival observed following treatment with Patritumab-DXd, as determined in the same assay. In some embodiments, the antigen-binding molecule achieves an increase in median survival of subjects having a cancer (e.g. in an in vivo model, e.g. of a HER3-expressing cancer) that is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or 10 times the median survival observed following treatment with Patritumab-DXd, as determined in the same assay. Median survival may be expressed in days from the start of the experiment, for subjects in the relevant treatment groups.

In some embodiments, the antigen-binding molecule of the present disclosure displays similar or decreased Fc-mediated binding to human PBMCs, as compared to Fc-mediated binding to human PBMCs displayed by Patritumab-DXd. In some embodiments, the antigen-binding molecule of the present disclosure binds to PBMCs, e.g. human PBMCs, with a KD value that is similar to or higher than the KD value with which Patritumab-DXd binds to the relevant cells, in a given assay. In some embodiments, the KD for binding of the antigen-binding molecule to PBMCs is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the KD for binding of Patritumab-DXd to cells of the same type, as determined in the same assay. In some embodiments, the KD for binding of the antigen-binding molecule to PBMCs is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or 10 times the KD for binding of Patritumab-DXd to PBMCs, as determined in the same assay. In some embodiments, the antigen-binding molecule demonstrates up to 95%, up to 94%, up to 93%, up to 92%, up to 91%, 90%, up to 89%, up to 88%, up to 87%, up to 86%, up to 85%, up to 84%, up to 83%, up to 82%, up to 81%, up to 80%, up to 79%, up to 78%, up to 77%, up to 76%, up to 75%, up to 74%, up to 73%, up to 72%, up to 71%, up to 70%, up to 69%, up to 68%, up to 67%, up to 66%, up to 65%, up to 64%, up to 63%, up to 62%, up to 61%, up to 60%, up to 59%, up to 58%, up to 57%, up to 56%, up to 55%, up to 54%, up to 53%, up to 52%, up to 51%, up to 50%, up to 49%, up to 48%, up to 47%, up to 46%, up to 45%, up to 44%, up to 43%, up to 42%, up to 41%, up to 40%, up to 39%, up to 38%, up to 37%, up to 36%, up to 35%, up to 34%, up to 33%, up to 32%, up to 31%, up to 30%, up to 29%, up to 28%, up to 27%, up to 26%, up to 25%, up to 24%, up to 23%, up to 22%, up to 21%, up to 20%, up to 19%, up to 18%, up to 17%, up to 16%, up to 15%, up to 14%, up to 13%, up to 12%, up to 11%, up to 10%, up to 9%, up to 8%, up to 7%, up to 6%, or up to 5% less binding to PBMCs, as compared to the binding of Patritumab-DXd to cells of the same type, as determined in the same assay.

In some embodiments, the antigen-binding molecule of the present disclosure displays similar or increased internalization into cells expressing HER3, as compared to internalization into said cells displayed by Patritumab-DXd. In some embodiments, the antigen-binding molecule is internalized into cells expressing HER3 to a level that is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the level of internalization of Patritumab-DXd into the relevant cells, as determined in the same assay. In some embodiments, the antigen-binding molecule is internalized into cells expressing HER3 to a level that is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or 10 times the level of internalization of Patritumab-DXd into the relevant cells, as determined in the same assay.

In some embodiments, the antigen-binding molecule of the present disclosure displays similar or decreased macropinocytosis-dependent uptake by megakaryocytes, as compared to macropinocytosis-dependent uptake by Patritumab-DXd. In some embodiments, the antigen-binding molecule demonstrates up to 95%, up to 94%, up to 93%, up to 92%, up to 91%, 90%, up to 89%, up to 88%, up to 87%, up to 86%, up to 85%, up to 84%, up to 83%, up to 82%, up to 81%, up to 80%, up to 79%, up to 78%, up to 77%, up to 76%, up to 75%, up to 74%, up to 73%, up to 72%, up to 71%, up to 70%, up to 69%, up to 68%, up to 67%, up to 66%, up to 65%, up to 64%, up to 63%, up to 62%, up to 61%, up to 60%, up to 59%, up to 58%, up to 57%, up to 56%, up to 55%, up to 54%, up to 53%, up to 52%, up to 51%, up to 50%, up to 49%, up to 48%, up to 47%, up to 46%, up to 45%, up to 44%, up to 43%, up to 42%, up to 41%, up to 40%, up to 39%, up to 38%, up to 37%, up to 36%, up to 35%, up to 34%, up to 33%, up to 32%, up to 31%, up to 30%, up to 29%, up to 28%, up to 27%, up to 26%, up to 25%, up to 24%, up to 23%, up to 22%, up to 21%, up to 20%, up to 19%, up to 18%, up to 17%, up to 16%, up to 15%, up to 14%, up to 13%, up to 12%, up to 11%, up to 10%, up to 9%, up to 8%, up to 7%, up to 6%, or up to 5% less macropinocytosis-dependent uptake by megakaryocytes, as compared to the macropinocytosis-dependent uptake of Patritumab-DXd by megakaryocytes, as determined in the same assay.

In some embodiments, the antigen-binding molecule of the present disclosure displays similar or reduced toxicity to subjects administered the antigen-binding molecule as compared to subjects administered Patritumab-DXd. In some embodiments, the antigen-binding molecule has a similar or improved toxicological profile as compared to Patritumab-DXd.

In some embodiments, the number/proportion of white blood cells/neutrophils/platelets/red blood cells in the peripheral blood of a subject following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and 1.4 times, ≥0.85 times and ≤1.3 times, ≥0.9 times and ≤1.2 times or 0.95 times and ≤1.1 times the number/proportion of such cells observed following treatment with Patritumab-DXd, as determined in the same assay. In some embodiments, the number/proportion of white blood cells/neutrophils/platelets/red blood cells in the peripheral blood of a subject following administration of an antigen-binding molecule according to the present disclosure is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the number/proportion of such cells observed following treatment with Patritumab-DXd, as determined in the same assay.

In some embodiments, the level of a correlate of interstitial lung disease following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the level of the relevant correlate in a subject that has been administered Patritumab-DXd, as determined in the same assay. In some embodiments, the level of a correlate of interstitial lung disease following administration of an antigen-binding molecule according to the present disclosure is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 0.01 times the level of the relevant correlate in a subject that has been administered Patritumab-DXd, as determined in the same assay.

In some embodiments, the level of a correlate of impairment of hepatic function (e.g. AST and/or ALT) in the peripheral blood of a subject following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the level observed in the peripheral blood of a subject following treatment with Patritumab-DXd, as determined in the same assay. In some embodiments, the level of a correlate of impairment of hepatic function (e.g. AST and/or ALT) in the peripheral blood of a subject following administration of an antigen-binding molecule according to the present disclosure is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the peripheral blood of a subject following treatment with Patritumab-DXd, as determined in the same assay.

In some embodiments, the level/concentration of a correlate of impairment of renal function (e.g. BUN and/or creatinine) in the peripheral blood of a subject following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or 0.95 times and ≤1.1 times the level/concentration observed in the peripheral blood of a subject following treatment with Patritumab-DXd, as determined in the same assay. In some embodiments, the level/concentration of a correlate of impairment of renal function (e.g. BUN and/or creatinine) in the peripheral blood of a subject following administration of an antigen-binding molecule according to the present disclosure is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 0.01 times the level/concentration observed in the peripheral blood of a subject following treatment with Patritumab-DXd, as determined in the same assay.

In some embodiments, the level of a correlate of lung function in a subject following administration of an antigen-binding molecule according to the present disclosure is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times or ≥0.95 times and ≤1.1 times the level of the relevant correlate in a subject that has been administered Patritumab-DXd, as determined in the same assay. In some embodiments, the level of a correlate of lung function in a subject following administration of an antigen-binding molecule according to the present disclosure is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or 10 times the level of the relevant correlate in a subject that has been administered Patritumab-DXd, as determined in the same assay.

### Producing the Antigen-Binding Molecules

Antigen-binding molecules according to the present disclosure may be prepared according to methods for the production of antibody-drug conjugates known to the skilled person.

Antigen-binding moieties according to the present disclosure may be prepared by chemical synthesis, e.g. liquid or solid phase synthesis. For example, peptides/polypeptides can be synthesised using the methods described in, for example, Chandrudu et al., Molecules (2013), 18: 4373-4388, which is hereby incorporated by reference in its entirety.

Alternatively, antigen-binding moieties according to the present disclosure may be produced by recombinant expression. Molecular biology techniques suitable for recombinant production of polypeptides are well known in the art, such as those set out in Green and Sambrook, Molecular Cloning: A Laboratory Manual (4^{th} Edition), Cold Spring Harbor Press, 2012, and in Nat Methods. (2008); 5(2): 135-146 both of which are hereby incorporated by reference in their entirety. Methods for the recombinant production of antigen-binding polypeptides are also described in Frenzel et al., Front Immunol. (2013); 4: 217 and Kunert and Reinhart, Appl Microbiol Biotechnol. (2016) 100: 3451-3461, both of which are hereby incorporated by reference in their entirety.

In some cases, the antigen-binding moieties of the present disclosure are comprised of more than one polypeptide chain. In such cases, production of the antigen-binding moiety may comprise transcription and translation of more than one polypeptide, and subsequent association of the polypeptide chains to form the antigen-binding moiety.

For recombinant production according to the present disclosure, any cell suitable for the expression of polypeptides may be used. The cell may be a prokaryote or eukaryote. In some embodiments, the cell is a prokaryotic cell, such as a cell of archaea or bacteria. In some embodiments, the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove. In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according to a system described in Zemella et al. Chembiochem (2015) 16(17): 2420-2431, which is hereby incorporated by reference in its entirety.

Production of antigen-binding moieties may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4^{th} Edition, incorporated by reference herein above). Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide(s), the polypeptide(s) of interest may be isolated. Any suitable method for separating proteins from cells known in the art may be used. In order to isolate the polypeptide, it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins. Other methods for distinguishing different proteins are known in the art, for example ion exchange chromatography and size chromatography. These may be used as an alternative to precipitation or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

Antigen-binding moieties according to the present disclosure may be conjugated to linker-payload moieties according to the present disclosure for the production of antigen-binding molecules according to the present disclosure by any suitable techniques, which are well known to the skilled person and routinely employed in the art. Such methods are described e.g. in Chudasama et al., Nature Chemistry, (2016), 8:114-119, Baah et al., Molecules. (2021) 26(10): 2943, and Walsh et al., Chem. Soc. Rev. (2021) 50:1305-1353, all of which are hereby incorporated by reference in their entirety.

Conjugation of antigen-binding moieties and linker-payload moieties and the purification of antigen-binding molecules produced by such conjugation can be performed e.g. as described in in Beck et al., (2017) Nat Rev Drug Discov 16: 315-337; Peters and Brown Biosci Rep (2015) 35: art:e00225; McCombs and Owen, The AAPS Journal (2015) 17: 339-351; Jackson, Org Process Res Dev (2016) 20: 852-866; or Olivier and Hurvitz, Antibody-Drug Conjugates: Fundamentals, Drug Development, and Clinical, (2016) Wiley. In preferred embodiments, antigen-binding molecules according to the present disclosure may be generated using conjugation employing metal-free click chemistry.

In some embodiments, the linker-payload moiety is conjugated to a polypeptide of the antigen-binding moiety. In some embodiments, the linker-payload moiety is conjugated to more than one polypeptide (e.g. two polypeptides) of the antigen-binding moiety.

In some embodiments, the linker-payload moiety is conjugated to the CH2 region of an antigen-binding moiety according to the present disclosure. In some embodiments, the linker-payload moiety is conjugated to each CH2 region of an antigen-binding moiety. In some embodiments, the linker-payload moiety is conjugated to the CH2-CH3 region of an antigen-binding moiety. In some embodiments, the linker-payload moiety is conjugated to each CH2-CH3 region of an antigen-binding moiety. In some embodiments, the linker-payload moiety is conjugated to the Fc region of an antigen-binding moiety. In some embodiments, the linker-payload moiety is conjugated each CH2-CH3 region of an Fc region of an antigen-binding moiety.

In some embodiments, the linker-payload moiety is conjugated to the antigen-binding moiety via linkage at N297 (EU numbering) of the Fc region/CH2-CH3-region/CH2 region. In some embodiments, the linker-payload moiety is conjugated via linkage to an N-glycan at N297 (EU numbering) of the Fc region/CH2-CH3-region/CH2 region.

In some embodiments, conjugation is performed by reaction of an azido group on the antigen-binding moiety with a bicyclo[6.1.0]nonyne (BCN) moiety on the linker-payload moiety.

In some embodiments, the antigen-binding moiety comprises a carbohydrate moiety comprising an azido group. In some embodiments, the antigen-binding moiety comprises a glycan (e.g. an N-glycan) comprising an azido group. In some embodiments, the antigen-binding moiety comprises a glycan (e.g. an N-glycan) comprising a 6-azido-6-deoxy-N-acetylgalactosamine (i.e. 6-azido-GalNAc) moiety. In some embodiments, the antigen-binding moiety comprises an CH2/CH2-CH3/Fc region comprising an N-glycan comprising a 6-azido-GalNAc residue. In some embodiments, the antigen-binding moiety comprises an CH2/CH2-CH3/Fc region comprising an N-glycan at N297 (EU numbering) comprising a 6-azido-GalNAc residue.

In some embodiments, an antigen-binding molecule according to the present disclosure is produced by a method employing GlycoConnect technology, which is described e.g. in van Geel et al., Bioconjug Chem. (2015) 26(11):2233-2242 and WO 2021/015622 A1, both of which are hereby incorporated by reference in their entirety. In some embodiments, an antigen-binding molecule according to the present disclosure is produced essentially as described in van Geel et al., Bioconjug Chem. (2015) 26(11):2233-2242.

In preferred embodiments, an antigen-binding molecule according to the present disclosure can be produced as described in WO 2022/058395 A1, which is incorporated by reference hereinabove. In particular, an antigen-binding molecule according to the present disclosure can be produced as described in paragraphs [0137] to [0174] of WO 2022/058395 A1, which are specifically incorporated by reference. In accordance with such methods described in WO 2022/058395 A1, it will be appreciated that ‘AB’ as referred to in paragraphs [0137] to [0174] of WO 2022/058395 A1 is an antigen-binding moiety as described herein.

In some embodiments, the method comprises contacting an antigen-binding moiety comprising an azido moiety with a compound having structure (F):

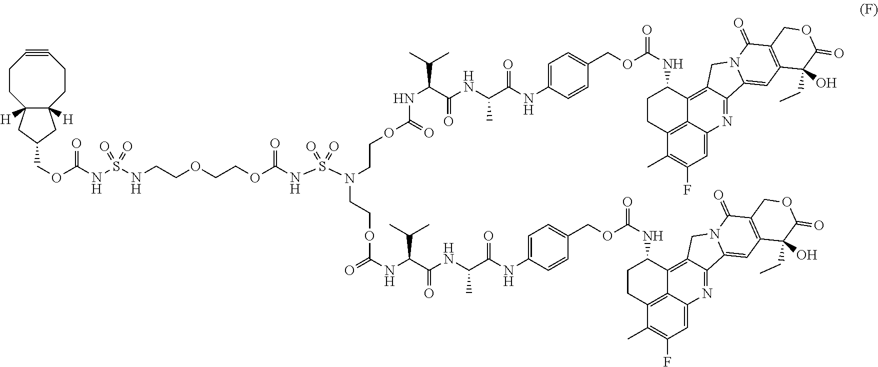 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)OCCN(CCOC(=O)N[C@H](C(=O)N[C@@H](C)C(=O)Nc2ccc(COC(=O)N[C@H]4CCc5c(C)c(F)cc6nc7c(c4c56)Cn4c-7cc5c(c4=O)COC(=O)[C@]5(O)CC)cc2)C(C)C)S(=O)(=O)NC(=O)OCCOCCNS(=O)(=O)NC(=O)OC[C@H]2C[C@H]4CCC#CCC[C@H]4C2)C(C)C)cc1)CC3]

In some embodiments, the method comprises:
(i) contacting an antigen-binding moiety according to the present disclosure comprising a CH2 region/CH2-CH3 region/Fc region with an endoglycosidase, a galactosyltransferase, and a 6-azido-6-deoxy-N-acetylgalactosamine moiety donor, and
(ii) contacting the product of step (i) with a compound having structure (F).

In some embodiments, the endoglycosidase is an endoglycosidase described in WO 2017/137459 A1 (hereby incorporated by reference in its entirety), e.g. EndoSH, EndoS2 or EndoS as described in WO 2017/137459 A1.

In some embodiments, the galactosyltransferase is a galactosyltransferase described in Boeggeman et al., Protein Expression and Purification (2003), 30(2):219-229 (hereby incorporated by reference in its entirety), e.g. β1,4-Galactosyltransferase (EC 2.4.1.38), or β1,4-Galactosyltransferase comprising the amino acid substitution Y289L.

It will be appreciated that a ‘6-azido-6-deoxy-N-acetylgalactosamine moiety donor’ refers to a compound capable of serving as a donor for a 6-azido-6-deoxy-N-acetylgalactosamine moiety. In the context of the present disclosure, the 6-azido-6-deoxy-N-acetylgalactosamine moiety donor serves as a donor for producing an antigen-binding molecule comprising an a 6-azido-6-deoxy-N-acetylgalactosamine moiety, for subsequent conjugation of a linker-payload moiety (e.g. via reaction with a linker-payload moiety comprising a BCN moiety). In some embodiments, the 6-azido-6-deoxy-N-acetylgalactosamine moiety donor is 6-azido-6-deoxy-UDP-N-acetylgalactosamine (i.e. UDP-6-azido-GalNAc) (see e.g. Mayer et al., Bioorg Med Chem Lett (2011) 21(4):1199-201).

In some embodiments, the method further comprises purifying/isolating the antigen-binding molecule (i.e. from unreacted precursors and/or by-products). In some embodiments, the antigen-binding molecule may be purified/isolated by chromatography, e.g. size-exclusion chromatography.

In some embodiments, an antigen-binding molecule according to the present disclosure is produced essentially as described in Example 2 herein. In some embodiments, conjugation of an antigen-binding moiety according to the present disclosure with a linker-payload moiety according to the present disclosure is performed essentially as described in Example 2.3 herein.

### Compositions

The present disclosure provides a composition comprising an antigen-binding molecule according to the present disclosure.

The antigen-binding molecules described herein may be formulated as pharmaceutical compositions or medicaments for clinical use and may comprise a pharmaceutically-acceptable carrier, diluent, excipient or adjuvant. Thus, the present disclosure provides a pharmaceutical composition/medicament comprising an antigen-binding molecule described herein.

The pharmaceutical compositions/medicaments of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23^{rd} Edition (2020), Academic Press.

Pharmaceutical compositions and medicaments of the present disclosure may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the antigen-binding molecule provided in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the pharmaceutical compositions/medicament is formulated for injection or infusion, e.g. into a blood vessel, tissue/organ of interest, or a tumor.

The present disclosure also provides methods for the production of pharmaceutically useful compositions, such methods of production may comprise one or more steps selected from:
producing an antigen-binding molecule described herein,
isolating/purifying an antigen-binding molecule described herein; and/or
mixing an antigen-binding molecule described herein with a pharmaceutically-acceptable carrier, adjuvant, excipient or diluent.

For example, a further aspect of the present disclosure relates to a method of formulating or producing a medicament or pharmaceutical composition for use in the treatment of a disease/condition (e.g. a disease/condition described herein), the method comprising formulating a pharmaceutical composition or medicament by mixing an antigen-binding molecule described herein with a pharmaceutically-acceptable carrier, adjuvant, excipient or diluent.

### Therapeutic and Prophylactic Applications

The antigen-binding molecules and compositions described herein find use in therapeutic and prophylactic intervention for disease, e.g. cancers.

It will be appreciated that the antigen-binding molecules and compositions of the present disclosure may be used for the treatment/prevention of any disease/condition that would derive therapeutic or prophylactic benefit from a reduction in the level/activity of HER3, or a reduction in the number or activity of cells comprising/expressing HER3.

For example, the disease/condition may be a disease/condition in which HER3, or cells expressing/overexpressing HER3 are pathologically-implicated, e.g. a disease/condition in which an increased level/activity of HER3, or an increase in the number/proportion of cells comprising/expressing HER3 is positively associated with the onset, development or progression of the disease/condition, and/or severity of one or more symptoms of the disease/condition. In some embodiments, an increased level/activity of HER3, or an increase in the number/proportion of cells comprising/expressing HER3 may be a risk factor for the onset, development or progression of the disease/condition.

The present disclosure provides an antigen-binding molecule or composition described herein for use in a method of medical treatment or prophylaxis. Also provided is an antigen-binding molecule or composition described herein for use in a method of treating or preventing a cancer (e.g. a cancer described herein). Also provided is the use of an antigen-binding molecule or composition described herein in the manufacture of a medicament for treating or preventing a cancer (e.g. a cancer described herein). Also provided is a method of treating or preventing a cancer (e.g. a cancer described herein) in a subject, comprising administering to a subject a therapeutically or prophylactically effective amount of an antigen-binding molecule or composition described herein.

The methods may be effective to reduce the development or progression of a cancer, alleviation of the symptoms of a cancer or reduction in the pathology of a cancer. The methods may be effective to prevent progression of the cancer, e.g. to prevent worsening of, or to slow the rate of development of, the cancer. In some embodiments, the methods may lead to an improvement in the cancer, e.g. a reduction in the symptoms of the cancer or reduction in some other correlate of the severity/activity of the cancer. In some embodiments, the methods may prevent development of the cancer to a later stage (e.g. a chronic stage or metastasis).

As used herein, a ‘cancer’ may be or comprise any unwanted cell proliferation (or any disease manifesting itself by unwanted cell proliferation), neoplasm or tumor. The cancer may be benign or malignant. The cancer may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, biliary tract, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue; examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

In some embodiments, the cancer to be treated/prevented comprises cells expressing an EGFR family member (e.g. HER3, EGFR, HER2 or HER4), and/or cells expressing a ligand for an EGFR family member. In some embodiments, the cancer to be treated/prevented comprises cells expressing a mutant or wildtype version of an EGFR family member (e.g. HER3, EGFR, HER2 or HER4). In some embodiments, the cancer to be treated/prevented is a cancer which is positive for an EGFR family member. In some embodiments, the cancer comprises cells that overexpress an EGFR family member and/or a ligand for an EGFR family member. Overexpression can be determined by detection of a level of expression which is greater than the level of expression by equivalent non-cancerous cells/non-tumor tissue.

Expression may be determined by any suitable means. Expression may be gene expression or protein expression. Gene expression can be determined e.g. by detection of mRNA encoding HER3, for example by quantitative real-time PCR (qRT-PCR). Protein expression can be determined e.g. by antibody-based methods, for example by western blot, immunohistochemistry, immunocytochemistry, flow cytometry, or ELISA.

In some embodiments the cancer is a cancer in which HER3 is pathologically-implicated. That is, in some embodiments the cancer is a cancer which is caused or exacerbated by the expression of HER3, a cancer for which expression of HER3 is a risk factor and/or a cancer for which expression of HER3 is positively associated with onset, development, progression, severity or metastasis of the cancer. The cancer may be characterised by expression of HER3, e.g. the cancer may comprise cells (e.g. cells of tumor tissue) expressing HER3. Such cancers may be referred to as being positive for HER3. A cancer which is ‘positive’ for HER3 may be a cancer comprising cells expressing HER3 (e.g. at the cell surface). A cancer which is ‘positive’ for HER3 may overexpress HER3.

In some embodiments, the cancer to be treated/prevented comprises cells harbouring a genetic variant (e.g. a mutation) which causes increased (gene and/or protein) expression and/or activity of HER3, relative to comparable cells harbouring a reference allele not comprising the genetic variant (e.g. a non-mutated, or ‘wildtype’ allele). The genetic variant may be or comprise insertion, deletion, substitution to, or larger-scale translocation/rearrangement of, the nucleotide sequence relative to the reference allele.

A mutation ‘resulting in’ increased expression of HER3 may be known or predicted to cause, or may be associated with, increased gene/protein expression of HER3. A mutation ‘resulting in’ increased activity of HER3 may be known or predicted to cause, or may be associated with, increased HER3-mediated signalling and/or EGFR-mediated signalling. Mutations resulting in increased expression and/or activity of HER3 may be referred to as ‘activating’ mutations.

A mutation which causes increased expression of HER3 may result in gene or protein expression of HER3 which is not expressed by, and/or not encoded by genomic nucleic acid of, an equivalent cell not harbouring the mutation. That is, the HER3 may be a neoantigen arising as a result of the mutation, and thus ‘increased expression’ may be from no expression.

A mutation which causes increased expression of HER3 may result in increased gene or protein expression of HER3 which is expressed by, and/or which is encoded by genomic nucleic acid of, an equivalent cell not comprising the mutation. By way of illustration, a cell may comprise a mutation resulting in an increase in the level of transcription of nucleic acid encoding HER3 relative to the level of transcription of nucleic acid encoding HER3 by an equivalent cell not comprising the mutation.

In some embodiments, a mutation which causes increased expression of HER3 may cause an increase in gene expression of HER3 relative to an equivalent cell not comprising the mutation. In some embodiments, a mutation which causes increased expression of HER3 may cause an increase in protein expression of HER3 relative to an equivalent cell not comprising the mutation.

In some embodiments, a mutation which causes increased expression of HER3 may cause an increase in the level of HER3 on or at the cell surface of a cell comprising the mutation, relative to an equivalent cell not comprising the mutation.

Cells having increased expression of HER3 relative to the level of expression of HER3 by a reference cell (e.g. as a result of mutation) may be described as ‘overexpressing’ HER3, or having ‘upregulated expression’ of HER3. For example, a cancer comprising cells harbouring a mutation resulting in increased expression of HER3 relative to equivalent cells lacking the mutation may be described as a cancer comprising cells displaying overexpression/upregulated expression of HER3. In some embodiments, the reference cell lacking the mutation may be a non-cancerous cell (e.g. of equivalent cell type) or a cancerous cell (e.g. of equivalent cancer type).

A mutation which causes increased activity of HER3 may result in an increase in HER3-mediated signalling relative to the level of HER3-mediated signalling by an equivalent cell not comprising the mutation.

In some embodiments, a cancer to be treated/prevented in accordance with the present disclosure may be characterised by an increase in the expression and/or activity of HER3 (i.e. gene and/or protein expression) in an organ/tissue/subject affected by the disease/condition e.g. as compared to normal organ/tissue/subject (i.e. in the absence of the disease/condition). In some embodiments, cells and/or a tumor of a cancer to be treated/prevented may be characterised by an increase in the expression and/or activity of HER3, e.g. as compared to the level of expression and/or activity observed in equivalent non-cancerous cells/non-tumor tissue.

A HER3-overexpressing cancer may overexpress HER3 as a consequence of amplification of the HER3 gene.

In some embodiments, a cancer to be treated/prevented in accordance with the present disclosure is a HER3-amplified cancer.

HER3 amplification can be identified using techniques well known in the art, such as in situ hybridisation.

For example, HER3 amplification can be evaluated by fluorescence in situ hybridisation, e.g. as described in Chung et al., J Gynecol Oncol. (2019) 30(5): e75. HER3-amplified cancers may comprise a ratio of 12q13.2 to chromosome 12 centromere ≥2, as determined by in situ hybridisation.

HER3 and its association with and role in cancer is reviewed e.g. in Mishra, et al., Oncol Rev. (2018) 12(1): 355, Karachaliou et al., BioDrugs. (2017) 31(1):63-73 and Zhang et al., Acta Biochimica et Biophysica Sinica (2016) 48(1): 39-48, all of which are hereby incorporated by reference in their entirety. Mishra, et al., Oncol Rev. (2018) 12(1): 355 also describes intervention targeting HER3 for the treatment of cancer, including monoclonal anti-HER3 antibody therapy.

In some embodiments, the cancer to be treated/prevented comprises cells expressing a ligand for HER3 (e.g. NRG1 and/or NRG2). In some embodiments, the cancer to be treated/prevented comprises cells expressing a level of expression of NRG1 and/or NRG2 which is greater than the level of expression by equivalent non-cancerous cells/non-tumor tissue. The cancer may be described as comprising cells that overexpress NRG1 and/or NRG2.

HER3-binding antigen-binding molecules described herein bind to HER3 with extremely high affinity when HER3 is bound by NRG (i.e. when HER3 is provided in the ‘open’ conformation), and also when HER3 is not bound by NRG (i.e. when HER3 is provided in the ‘closed’ conformation). Thus, they are particularly useful for the treatment/prevention of cancers characterised by HER3 ligand expression/overexpression, for example cancers/tumors comprising cells expressing/overexpressing a ligand for HER3.

In some embodiments, the cancer to be treated/prevented comprises cells harbouring a genetic variant (e.g. a mutation) which causes increased (gene and/or protein) expression of a ligand for HER3, relative to comparable cells harbouring a reference allele not comprising the genetic variant (e.g. a non-mutated, or ‘wildtype’ allele). The genetic variant may be or comprise insertion, deletion, substitution to, or larger-scale translocation/rearrangement of, the nucleotide sequence relative to the reference allele.

A mutation ‘resulting in’ increased expression of a ligand for HER3 may be known or predicted to cause, or may be associated with, increased gene/protein expression of a ligand for HER3. Mutations resulting in increased expression of a ligand for HER3 may be referred to as ‘activating’ mutations.

A mutation which causes increased expression of a ligand for HER3 may result in gene or protein expression of a ligand for HER3 which is not expressed by, and/or not encoded by genomic nucleic acid of, an equivalent cell not harbouring the mutation. That is, the ligand for HER3 may be a neoantigen arising as a result of the mutation, and thus ‘increased expression’ may be from no expression. By way of illustration, a cell comprising CD74-NRG1 gene fusion displays increased expression of the CD74-NRG1 fusion polypeptide encoded by the gene fusion relative to cells lacking the CD74-NRG1 gene fusion.

A mutation which causes increased expression of a ligand for HER3 may result in increased gene or protein expression of a ligand for HER3 which is expressed by, and/or which is encoded by genomic nucleic acid of, an equivalent cell not comprising the mutation. By way of illustration, a cell may comprise a mutation resulting in an increase in the level of transcription of nucleic acid encoding NRG1 relative to level of transcription of nucleic acid encoding NRG1 by an equivalent cell not comprising the mutation.

In some embodiments, a mutation which causes increased expression of a ligand for HER3 may cause an increase in gene expression of a ligand for HER3 relative to an equivalent cell not comprising the mutation. In some embodiments, a mutation which causes increased expression of a ligand for HER3 may cause an increase in protein expression of a ligand for HER3 relative to an equivalent cell not comprising the mutation.

In some embodiments, a mutation which causes increased expression of a ligand for HER3 may cause an increase in the level of a ligand for HER3 on or at the cell surface of a cell comprising the mutation, relative to an equivalent cell not comprising the mutation. In some embodiments, a mutation which causes increased expression of a ligand for HER3 may cause an increase in the level of a secretion of a ligand for HER3 from a cell comprising the mutation, relative to an equivalent cell not comprising the mutation.

Cells having increased expression of a ligand for HER3 relative to the level of expression of the ligand for HER3 by a reference cell (e.g. as a result of mutation) may be described as ‘overexpressing’ the ligand for HER3, or having ‘upregulated expression’ of the ligand for HER3. For example, a cancer comprising cells harbouring a mutation resulting in increased expression of a ligand for HER3 relative to equivalent cells lacking the mutation may be described as a cancer comprising cells displaying overexpression/upregulated expression of the ligand for HER3. In some embodiments, the reference cell lacking the mutation may be a non-cancerous cell (e.g. of equivalent cell type) or a cancerous cell (e.g. of equivalent cancer type).

Herein, a ‘ligand for HER3’ is generally intended to refer to a molecule capable of binding to HER3 through the ligand binding region of HER3 formed by domains I and III of HER3. In some embodiments, a ligand for HER3 binds to HER3 via interaction with domains I and/or III of HER3. Exemplary ligands for HER3 include neuregulins such as NRG1 and NRG2, which bind to HER3 via interaction between their EGF-like domains and the ligand binding region of HER3.

The HER3 ligand is preferably able to bind and trigger signalling through the HER3 receptor and/or receptor complexes comprising HER3. As will be clear from the present disclosure, receptor complexes comprising HER3 may further comprise an interaction partner for HER3 as described herein, e.g. HER3, HER2, EGFR, HER4, HGFR, IGF1R and/or cMet.

In some embodiments the ligand for HER3 is able to bind to HER3 receptor/receptor complex expressed by a cell other than the cell having increased expression of the HER3 ligand. For example, in some embodiments the ligand for HER3 is able to bind to a HER3-expressing cancer cell.

In some embodiments the ligand for HER3 is able to bind to HER3 receptor/receptor complex expressed by the cell having increased expression of the HER3 ligand.

In some embodiments the cancer to be treated/prevented comprises (i) cells expressing HER3, and (ii) cells expressing a ligand for HER3 (e.g. having increased expression of a ligand for HER3, e.g. as a consequence of mutation resulting in increased expression of a ligand for HER3).

In some embodiments the cancer to be treated/prevented comprises cells which (i) express HER3 and (ii) which also express a ligand for HER3 (e.g. which have increased expression of a ligand for HER3, e.g. as a consequence of mutation resulting in increased expression of a ligand for HER3).

In some embodiments, the ligand for HER3 comprises, or consists of, the amino acid sequence of a HER3-binding region of a ligand for HER3, or an amino acid sequence derived from a HER3-binding region of a ligand for HER3. An amino acid sequence which is derived from a HER3-binding region of a ligand for HER3 may comprise at least 60% (e.g. 70%, 75%, 80%, 85%, 90%, 91%, 92%, 93%, 94%, 95%, 96%, 97%, 98%, 99% or 100%) amino acid sequence identity to the amino acid sequence from which it is derived.

In some embodiments, the ligand for HER3 comprises an EGF-like domain capable of binding to HER3, or a HER3-binding fragment thereof. In some embodiments, a HER3-binding EGF-like domain/fragment is, or is derived from, an EGF family member (e.g. heparin-binding EGF-like growth factor (HB-EGF), transforming growth factor-α (TGF-α), amphiregulin (AR), epiregulin (EPR), epigen, betacellulin (BTC), NRG1, NRG2, NRG3 or NRG4).

Exemplary ligands for HER3 include neuregulins (NRGs). Neuregulins include NRG1 (including alpha, alpha2b, and alpha3 isoforms thereof), NRG2, NRG3 and NRG4. In some embodiments, an NRG is selected from NRG1, NRG2, NRG3 and NRG4. In some embodiments, an NRG is selected from NRG1 and NRG2.

The EGF-like domain of human NRG1, through which it binds to HER3, is formed by positions 178-222 of UniProt:Q02297-1. The EGF-like domain of human NRG2 is formed by positions 341-382 of UniProt:O14511-1. The EGF-like domain of human NRG3 is formed by positions 286-329 of UniProt:B9EGV5-1. The EGF-like domain of human NRG4 is formed by positions 5-46 of UniProt:Q8WWG1-1. In some embodiments, an EGF-like domain/fragment comprises, or consists of, an amino acid sequence having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG (NRG1, NRG2, NRG3 or NRG4).

In some embodiments a ligand for HER3 is not an EGFR family protein (e.g. HER3, HER2, EGFR, HER4, HGFR, IGF1R, cMet).

In some embodiments, the mutation resulting in increased expression of a ligand for HER3 is an NRG gene fusion. In some embodiments, the ligand for HER3 is the product of (i.e. a polypeptide encoded by) an NRG gene fusion. In some embodiments the cancer comprises cells having an NRG gene fusion. As used herein, an ‘NRG gene fusion’ refers to a genetic variant encoding a polypeptide comprising (i) an amino acid sequence of an NRG protein (e.g. NRG1, NRG2, NRG3 or NRG4; e.g. NRG1 or NRG2), and (ii) an amino acid sequence of a protein other than the NRG protein.

It will be appreciated that an NRG gene fusion preferably encodes a HER3 ligand as described herein. In some embodiments, an NRG gene fusion encodes a polypeptide comprising a HER3-binding region of an NRG protein. In some embodiments, an NRG gene fusion encodes a polypeptide comprising the EGF-like domain of an NRG protein, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG protein.

In some embodiments, an NRG gene fusion encodes a fusion polypeptide comprising a transmembrane domain. In some embodiments, an NRG gene fusion encodes a fusion polypeptide comprising the transmembrane domain of a protein other than the NRG protein.

In some embodiments, an NRG gene fusion is an NRG1 gene fusion. In some embodiments, the NRG1 gene fusion encodes a polypeptide comprising the EGF-like domain of NRG1, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of NRG1.

NRG1 gene fusions are described e.g. in WO 2021/048274 A1, WO 2018/182422 A1, WO 2019/051155 A1, Dhanasekaran et al., Nat Commun. (2014) 5: 5893, Drilon et al., Cancer Discov. (2018) 8(6):686-695, Nagasaka et al., Journal of Thoracic Oncology (2019) 14(8):1354-1359 and Jonna et al., Clin Cancer Res. (2019) 25(16):4966-4972, all of which are hereby incorporated by reference in their entirety. The diversity of NRG1 gene fusions may result from NRG1 being located on chromosome 8, which is particularly susceptible to genomic translocation events (Adelaide et al., Genes Chromosomes Cancer. (2003)37(4):333-45).

In some embodiments, an NRG1 gene fusion is selected from CLU-NRG1, CD74-NRG1, DOC4-NRG1, SLC3A2-NRG1, RBPMS-NRG1, WRN-NRG1, SDC4-NRG1, RAB21L1-NRG1, VAMP2-NRG1, KIF13B-NRG1, THAP7-NRG1, SMAD4-NRG1, MDK-NRG1, TNC-NRG1, DIP2B-NRG1, MRPL13-NRG1, PARP8-NRG1, ROCK1-NRG1, DPYSL2-NRG1, ATP1B1-NRG1, CDH6-NRG1, APP-NRG1, AKAP13-NRG1, THBS1-NRG1, FOXA1-NRG1, PDE7A-NRG1, RAB31L1-NRG1, CDK1-NRG1, BMPRIB-NRG1, TNFRSF10B-NRG1, and MCPH1-NRG1. In some embodiments, an NRG1 gene fusion is CLU-NRG1. CD74-NRG1 gene fusion is described e.g. in Fernandez-Cuesta et al. Cancer Discov. (2014) 4:415-22 and Nakaoku et al., Clin Cancer Res (2014) 20:3087-93. DOC4-NRG1 gene fusion is described e.g. in Liu et al., Oncogene. (1999) 18(50):7110-4 and Wang et al., Oncogene. (1999) 18(41):5718-21. SLC3A2-NRG1 gene fusion is described e.g. in Nakaoku et al., Clin Cancer Res (2014) 20:3087-93, Shin et al., Oncotarget (2016) 7:69450-65 and Shin et al., Mol Cancer Ther. (2018) 17(9):2024-2033. RBPMS-NRG1, WRN-NRG1, RAB21L1-NRG1 and SDC4-NRG1 gene fusions are described e.g. in Dhanasekaran et al., Nat Commun. (2014) 5: 5893. VAMP2-NRG1 gene fusion is described e.g. in Jung et al., J Thorac Oncol. (2015) 10(7):1107-11 and Shim et al., J Thorac Oncol. (2015) 10(8):1156-62. KlF13B-NRG1 gene fusion is described e.g. in Xia et al., Int J Surg Pathol. (2017) 25(3):238-240. SMAD4-NRG1, AKAP13-NRG1, THBS1-NRG1, FOXA1-NRG1, PDE7A-NRG1, RAB31L1-NRG1 and THAP7-NRG1 gene fusions are described e.g. in Drilon et al., Cancer Discov. (2018) 8(6):686-695. MDK-NRG1, TNC-NRG1, DIP2B-NRG1, MRPL13-NRG1, PARP8-NRG1, ROCK1-NRG1 and DPYSL2-NRG1 gene fusions are described e.g. in Jonna et al., Clin Cancer Res. (2019) 25(16):4966-4972. ATP1B1-NRG1 gene fusion is described e.g. in Drilon et al., Cancer Discov. (2018) 8(6):686-695 and Jones et al., Annals of Oncology (2017)28:3092-3097. CLU-NRG1 gene fusion is described e.g. in Drilon et al., Cancer Discov. (2018) 8(6):686-695 and Nagasaka et al., Journal of Thoracic Oncology (2019) 14(8):1354-1359.

In some embodiments, an NRG gene fusion is an NRG2 gene fusion. In some embodiments, the NRG2 gene fusion encodes a polypeptide comprising the EGF-like domain of NRG2, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of NRG2.

NRG2 gene fusions include SLC12A2-NRG2 described e.g. in WO 2021/048274 A1, WO 2015/093557 A1, and ZNF208-NRG2 described in Dupain et al., Mol Ther. (2019) 27(1):200-218.

A cancer comprising cells having a mutation which results in increased expression of a ligand for HER3 (e.g. comprising cells having an NRG gene fusion, e.g. an NRG1 gene fusion or an NRG2 gene fusion) can be any cancer described herein. In some embodiments, such cancer may be of tissues/cells derived from the lung, breast, head, neck, kidney, ovary, pancreas, prostate, uterus, gallbladder, biliary tract, colon, rectum, bladder, soft tissue or nasopharynx.

In some embodiments, a cancer comprising cells having a mutation which results in increased expression of a ligand for HER3 (e.g. comprising cells having an NRG gene fusion, e.g. an NRG1 gene fusion or an NRG2 gene fusion) is selected from: lung cancer, non-small cell lung cancer, lung adenocarcinoma, invasive mucinous lung adenocarcinoma, lung squamous cell carcinoma, breast cancer, breast carcinoma, breast invasive carcinoma, head and neck cancer, head and neck squamous cell carcinoma, renal cancer, renal clear cell carcinoma, ovarian cancer, ovarian serous cystadenocarcinoma, pancreatic cancer, pancreatic adenocarcinoma, pancreatic ductal adenocarcinoma, prostate cancer, prostate adenocarcinoma, endometrial cancer, uterine carcinosarcoma, gallbladder cancer, biliary tract cancer, cholangiocarcinoma, colorectal cancer, metastatic colorectal cancer, bladder cancer, urothelial bladder cancer, sarcoma, soft tissue sarcoma, neuroendocrine tumor, neuroendocrine tumor of the nasopharynx and homologous recombination deficiency (HRD) cancer.

In some embodiments, the cancer to be treated/prevented is lung cancer (e.g. non-small cell lung cancer, lung adenocarcinoma, invasive mucinous lung adenocarcinoma or lung squamous cell carcinoma) comprising cells having an NRG1 gene fusion.

It will be appreciated that in embodiments herein, cancers comprising cells having specified characteristics may be or comprise tumors comprising cells having those characteristics.

As is common in the art, a cancer/tumor comprising cells having specified characteristics may be referred to herein simply as a cancer/tumor having those characteristics. By way of illustration, a cancer/tumor comprising cells having an NRG1 gene fusion may be referred to simply as ‘a cancer/tumor comprising NRG1 gene fusion’, or ‘an NRG1 gene fusion cancer/tumor’.

In some embodiments, the cancer to be treated/prevented comprises mutation conferring resistance to treatment with an inhibitor of BRAF. In some embodiments, the mutation is mutation at BRAF V600. In some embodiments, the mutation is BRAF V600E or V600K. The cancer may be thyroid or colon cancer, e.g. RAS wildtype colorectal cancer. In some embodiments, the cancer to be treated/prevented comprises mutation conferring resistance to treatment with an inhibitor of BRAF (e.g. mutation at BRAF V600), and the treatment comprises administration of vemurafenib or darafenib.

In some embodiments, the cancer may be a relapsed cancer. As used herein, a ‘relapsed’ cancer refers to a cancer which responded to a treatment (e.g. a first line therapy for the cancer), but which has subsequently re-emerged/progressed, e.g. after a period of remission. For example, a relapsed cancer may be a cancer whose growth/progression was inhibited by a treatment (e.g. a first line therapy for the cancer), and which has subsequently grown/progressed.

In some embodiments, the cancer may be a refractory cancer. As used herein, a ‘refractory’ cancer refers to a cancer which has not responded to a treatment (e.g. a first line therapy for the cancer). For example, a refractory cancer may be a cancer whose growth/progression was not inhibited by a treatment (e.g. a first line therapy for the cancer). In some embodiments a refractory cancer may be a cancer for which a subject receiving treatment for the cancer did not display a partial or complete response to the treatment.

In some embodiments, a cancer is selected from: a cancer comprising cells expressing/overexpressing an EGFR family member (e.g. HER3, EGFR, HER2 or HER4), a cancer comprising cells expressing/overexpressing HER3, a cancer comprising cells having a mutation resulting in increased expression of a ligand for HER3, a cancer comprising cells having an NRG gene fusion, a solid tumor, a hematological cancer, a squamous cell cancer, breast cancer, breast carcinoma, breast invasive carcinoma, ductal carcinoma, metastatic breast cancer, triple-negative breast cancer, HER2-positive breast cancer, HER2-negative breast cancer, hormone receptor-positive breast cancer, HER2-negative/hormone receptor-positive breast cancer (e.g. a cancer expressing estrogen receptor (ER) and/or progesterone receptor (PR)), gastric cancer, gastric carcinoma, gastric adenocarcinoma, gastrointestinal adenocarcinoma, colorectal cancer, metastatic colorectal cancer, colon cancer, colorectal carcinoma, colorectal adenocarcinoma, colon adenocarcinoma, head and neck cancer, head and neck squamous cell carcinoma (HNSCC), lung cancer, non-small cell lung cancer, lung adenocarcinoma, invasive mucinous lung adenocarcinoma, lung squamous cell carcinoma (LUSC), ovarian cancer, ovarian carcinoma, ovarian serous adenocarcinoma, ovarian serous cystadenocarcinoma, fallopian tube cancer, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, oral cavity cancer, oropharyngeal cancer, esophageal cancer, esophageal squamous cell carcinoma (ESCC), esophageal adenocarcinoma, liver cancer, hepatocellular carcinoma, cholangiocarcinoma, gallbladder cancer, biliary tract cancer, uterine cancer, endometrial cancer, uterine corpus endometrial carcinoma, uterine carcinosarcoma, thyroid cancer, thyroid carcinoma, pheochromocytoma, paraganglioma, bladder cancer, bladder urothelial carcinoma, prostate cancer, prostate adenocarcinoma, castration-resistant prostate cancer, metastatic prostate cancer, metastatic castration-resistant prostate cancer, retinoblastoma, sarcoma, soft tissue sarcoma, peritoneal cancer, thymoma, neuroendocrine tumor, neuroendocrine tumor of the nasopharynx and homologous recombination deficiency (HRD) cancer (e.g. HRD ovarian cancer, HRD breast cancer, HRD prostate cancer or HRD pancreatic cancer).

In some embodiments, a cancer is selected from: colorectal cancer, colorectal carcinoma, prostate cancer, prostate carcinoma, lung cancer, lung adenocarcinoma, breast cancer, breast carcinoma, breast invasive carcinoma, ovarian cancer, ovarian adenocarcinoma, ovarian serous adenocarcinoma, gastric cancer and melanoma.

Treatment of a cancer in accordance with the methods of the present disclosure achieves one or more of the following treatment effects: reduces the number of cancer cells in the subject, reduces the size of a cancerous tumor/lesion in the subject, inhibits (e.g. prevents or slows) growth of cancer cells in the subject, inhibits (e.g. prevents or slows) growth of a cancerous tumor/lesion in the subject, inhibits (e.g. prevents or slows) the development/progression of a cancer (e.g. to a later stage, or metastasis), reduces the severity of symptoms of a cancer in the subject, increases survival of the subject (e.g. progression free survival or overall survival), reduces a correlate of the number or activity of cancer cells in the subject, and/or reduces cancer burden in the subject.

Subjects may be evaluated in accordance with the Revised Criteria for Response Assessment: The Lugano Classification (described e.g. in Cheson et al., J Clin Oncol (2014) 32: 3059-3068, incorporated by reference hereinabove) in order to determine their response to treatment. In some embodiments, treatment of a subject in accordance with the methods of the present disclosure achieves one of the following: complete response, partial response, or stable disease.

Prevention may refer to prevention of development of a cancer, and/or prevention of worsening of a cancer, e.g. prevention of progression of a cancer, e.g. to a later stage (e.g. metastasis).

In some embodiments, administration of an antigen-binding molecule/composition according to the present disclosure may be associated with one or more of: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of one or more symptoms of the cancer, a reduction in the number of cancer cells, a reduction in the cancer burden, a reduction in tumour size/volume, and/or an increase in survival of subjects having the cancer (e.g. progression free survival or overall survival).

In accordance with various aspects of the present disclosure, a method of treating and/or preventing a cancer according to the present disclosure may comprise inhibiting the growth of a tumor, reducing the size/volume of a tumor and/or increasing the survival of a subject having the cancer.

In accordance with various aspects of the present disclosure, methods are provided which are for, or which comprise (e.g. in the context of treatment/prevention of a cancer, e.g. a cancer described herein), one or more of the following:
binding to cells expressing HER3;
inhibiting the proliferation of HER3-expressing cells;
killing cells expressing HER3;
inhibiting tumor growth and/or reducing tumor size/volume, e.g. of a HER3-expressing cancer; and/or
increasing the survival of subjects having a cancer, e.g. a HER3-expressing cancer.

Also provided are antigen-binding molecules and compositions according to the present disclosure for use in such methods, and the use of antigen-binding molecules and compositions according to the present disclosure in manufacture of compositions (e.g. medicaments) for use in such methods. It will be appreciated that the methods typically comprise administering an antigen-binding molecule according to the present disclosure to a subject.

Similarly, one or more of the following may be observed in a subject following therapeutic or prophylactic intervention in accordance with the present disclosure (e.g. compared to the level/number/proportion etc. prior to intervention):
inhibition of proliferation of HER3-expressing cells;
killing of cells expressing HER3;
inhibition of tumor growth and/or reduction of tumor size/volume, e.g. of a HER3-expressing cancer; and/or
increased survival of a subject having a cancer, e.g. a HER3-expressing cancer.

In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

Administration of the antigen-binding molecules and compositions of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (Ed. A. Adejare), 23^{rd} Edition (2020), Academic Press.

Administration of the antigen-binding molecules and compositions of the present disclosure may be e.g. parenteral, systemic, topical, intracavitary, intravascular, intravenous, intra-arterial, intramuscular, intrathecal, intraocular, intraconjunctival, intratumoral, subcutaneous, intradermal, oral or transdermal. Administration may be by injection, infusion or ingestion.

In some aspects and embodiments, articles of the present disclosure may be administered to a tissue/organ of interest (e.g. a tissue/organ affected by the disease/condition affected by the condition (e.g. a tissue/organ in which symptoms of the disease/condition manifest). In some aspects and embodiments, articles of the present disclosure may be administered to the blood (i.e. intravenous/intra-arterial administration) by injection or infusion (e.g. via cannula), or may be administered subcutaneously or orally. In some aspects and embodiments, articles of the present disclosure may be administered to a tumor.

In some embodiments, therapeutic or prophylactic intervention according to the present disclosure may further comprise administering another agent for the treatment/prevention of the relevant disease/condition. Administration of antigen-binding molecules and compositions described herein may be alone or in combination with other treatments, either simultaneously or sequentially dependent upon the condition to be treated. Simultaneous administration refers to administration with another therapeutic agent together, for example as a pharmaceutical composition containing both agents (combined preparation), or immediately after each other (e.g. within 1, 4, 6, 8 or 12 hours) and optionally via the same route of administration (e.g. to the same tissue, artery, vein or other blood vessel). Sequential administration refers to administration of one agent followed after a given time interval by separate administration of another agent. It is not required that the two agents are administered by the same route, although this is the case in some embodiments. The time interval may be any time interval.

Multiple doses of the antigen-binding molecules and compositions may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or31 days, or1, 2, 3, 4, 5, or 6 months. By way of example, doses may be given once every 7, 14, 21 or 28 days (plus or minus 3, 2, or 1 days).

### Methods of Detection

The present disclosure also provides the articles of the present disclosure for use in methods for detecting, localising or imaging HER3, or cells expressing HER3. The present disclosure provides an antigen-binding molecule or composition described herein for use in a method of diagnosis or prognosis of a disease/condition, e.g. a disease/condition described herein.

The antigen-binding molecules described herein may be used in methods that involve detecting binding of the antigen-binding molecule to HER3. Such methods may involve detection of the bound complex of the antigen-binding molecule and HER3. It will be appreciated that the HER3 may be HER3 expressed by a cell, e.g. in or at the cell surface of a cell expressing HER3.

As such, a method is provided, comprising contacting a sample containing, or suspected to contain, HER3, and detecting the formation of a complex of the antigen-binding molecule and HER3. Also provided is a method comprising contacting a sample containing, or suspected to contain, a cell expressing HER3, and detecting the formation of a complex of the antigen-binding molecule and a cell expressing HER3.

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, 75icrop-detectable label, radiolabel, chemical, nucleic acid or enzymatic label as described herein. Detection techniques are well known to those of skill in the art and can be selected to correspond with the labelling agent.

Methods comprising detecting HER3, or cells expressing HER3, include methods for diagnosing/prognosing a disease/condition described herein.

Methods of this kind may be performed in vitro on a patient sample, or following processing of a patient sample. Once the sample is collected, the patient is not required to be present for the in vitro method to be performed, and therefore the method may be one which is not practised on the human or animal body. In some embodiments, the method is performed in vivo.

Such methods may involve detecting or quantifying HER3 and/or cells expressing HER3, e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Detection in a sample may be used for the purpose of diagnosis of a disease/condition (e.g. a cancer), predisposition to a disease/condition, or for providing a prognosis (prognosticating) for a disease/condition, e.g. a disease/condition described herein. The diagnosis or prognosis may relate to an existing (previously diagnosed) disease/condition.

A sample may be taken from any tissue or bodily fluid. The sample may comprise or may be derived from: a tumor/biopsy thereof; a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition). In some embodiments, the sample may be obtained or derived from a tumor.

A subject may be selected for diagnostic/prognostic evaluation based on the presence of symptoms indicative of a disease/condition described herein, or based on the subject being considered to be at risk of developing a disease/condition described herein.

The present disclosure also provides methods for selecting/stratifying a subject for treatment with a HER3-targeted agent. In some embodiments a subject is selected for treatment/prevention in accordance with the methods of the present disclosure, or is identified as a subject which would benefit from such treatment/prevention, based on detection/quantification of HER3, or cells expressing HER3, e.g. in a sample obtained from the individual.

### Subjects

The subject in accordance with aspects described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer, e.g. a cancer described herein), may be suspected of having such a disease/condition, or may be at risk of developing/contracting such a disease/condition.

In some embodiments, the subject to be treated according to a therapeutic or prophylactic method of the present disclosure herein is a subject having, or at risk of developing, a cancer, e.g. a cancer described herein. In embodiments according to the present disclosure, a subject may be selected for treatment according to the methods based on characterisation for certain markers of such disease/condition.

In some embodiments, a patient may be selected for treatment described herein based on the detection of a cancer expressing/overexpressing HER3, e.g. in a sample obtained from the subject (e.g. a biopsy, e.g. of a tumor).

### Kits

The present disclosure also provides kits of parts. A kit according to the present disclosure may comprise components for performing a method described herein, in whole or in part.

The kit may have at least one container having a predetermined quantity of an antigen-binding molecule or composition described herein.

In some aspects of the present disclosure a kit of parts is provided. In some embodiments, the kit may comprise an antigen-binding molecule or composition described herein, and which may be provided in a predetermined quantity.

The kit may provide an antigen-binding molecule or composition described herein together with instructions for administration to a patient in order to treat a specified disease/condition (e.g. a disease/condition described herein, e.g. a cancer).

The kit may provide an antigen-binding moiety according to the disclosure, and a linker-payload moiety according to the present disclosure. The kit may further comprise reagents for conjugating the antigen-binding moiety and the linker-payload moiety.

The kit may further comprise reagents, buffers and/or standards required for execution of a method according to the present disclosure. Kits according to the present disclosure may include instructions for use, e.g. in the form of an instruction booklet or leaflet. The instructions may include a protocol for performing any one or more of the methods described herein.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Soding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

### Sequences

| SEQ | | |
|---|---|---|
| ID NO: | DESCRIPTION | SEQUENCE |
| 1 | Human HER3 | MRANDALQVLGLLFSLARGSEVGNSQAVCPGTLNGLSVTGDAENQYQTLYKLYERCEVVMGNLEIVLTGHN |
| | isoform 1 | ADLSFLQWIREVTGYVLVAMNEFSTLPLPNLRVVRGTQVYDGKFAIFVMLNYNTNSSHALRQLRLTQLTEILS |
| | (UniProt: | GGVYIEKNDKLCHMDTIDWRDIVRDRDAEIVVKDNGRSCPPCHEVCKGRCWGPGSEDCQTLTKTICAPQCN |
| | P21860-1, v1) | GHCFGPNPNQCCHDECAGGCSGPQDTDCFACRHFNDSGACVPRCPQPLVYNKLTFQLEPNPHTKYQYGG |
| | | VCVASCPHNFVVDQTSCVRACPPDKMEVDKNGLKMCEPCGGLCPKACEGTGSGSRFQTVDSSNIDGFVN |
| | | CTKILGNLDFLITGLNGDPWHKIPALDPEKLNVFRTVREITGYLNIQSWPPHMHNFSVFSNLTTIGGRSLYNRG |
| | | FSLLIMKNLNVTSLGFRSLKEISAGRIYISANRQLCYHHSLNWTKVLRGPTEERLDIKHNRPRRDCVAEGKVC |
| | | DPLCSSGGCWGPGPGQCLSCRNYSRGGVCVTHCNFLNGEPREFAHEAECFSCHPECQPMEGTATCNGS |
| | | GSDTCAQCAHFRDGPHCVSSCPHGVLGAKGPIYKYPDVQNECRPCHENCTQGCKGPELQDCLGQTLVLIG |
| | | KTHLTMALTVIAGLVVIFMMLGGTFLYWRGRRIQNKRAMRRYLERGESIEPLDPSEKANKVLARIFKETELRK |
| | | LKVLGSGVFGTVHKGVWIPEGESIKIPVCIKVIEDKSGRQSFQAVTDHMLAIGSLDHAHIVRLLGLCPGSSLQL |
| | | VTQYLPLGSLLDHVRQHRGALGPQLLLNWGVQIAKGMYYLEEHGMVHRNLAARNVLLKSPSQVQVADFGV |
| | | ADLLPPDDKQLLYSEAKTPIKWMALESIHFGKYTHQSDVWSYGVTVWELMTFGAEPYAGLRLAEVPDLLEK |
| | | GERLAQPQICTIDVYMVMVKCWMIDENIRPTFKELANEFTRMARDPPRYLVIKRESGPGIAPGPEPHGLTNK |
| | | KLEEVELEPELDLDLDLEAEEDNLATTTLGSALSLPVGTLNRPRGSQSLLSPSSGYMPMNQGNLGESCQES |
| | | AVSGSSERCPRPVSLHPMPRGCLASESSEGHVTGSEAELQEKVSMCRSRSRSRSPRPRGDSAYHSQRHS |
| | | LLTPVTPLSPPGLEEEDVNGYVMPDTHLKGTPSSREGTLSSVGLSSVLGTEEEDEDEEYEYMNRRRRHSPP |
| | | HPPRPSSLEELGYEYMDVGSDLSASLGSTQSCPLHPVPIMPTAGTTPDEDYEYMNRQRDGGGPGGDYAA |
| | | MGACPASEQGYEEMRAFQGPGHQAPHVHYARLKTLRSLEATDSAFDNPDYWHSRLFPKANAQRT |
| 2 | Human HER3 | MRANDALQVLGLLFSLARGSEVGNSQAVCPGTLNGLSVTGDAENQYQTLYKLYERCEVVMGNLEIVLTGHN |
| | isoform 2 | ADLSFLQWIREVTGYVLVAMNEFSTLPLPNLRVVRGTQVYDGKFAIFVMLNYNTNSSHALRQLRLTQLTGQF |
| | (UniProt: | PMVPSGLTPQPAQDWYLLDDDPRLLTLSASSKVPVTLAAV |
| | P21860-2) | |
| 3 | Human HER3 | MRANDALQVLGLLFSLARGSEVGNSQAVCPGTLNGLSVTGDAENQYQTLYKLYERCEVVMGNLEIVLTGHN |
| | isoform 3 | ADLSFLQWIREVTGYVLVAMNEFSTLPLPNLRVVRGTQVYDGKFAIFVMLNYNTNSSHALRQLRLTQLTEILS |
| | (UniProt: | GGVYIEKNDKLCHMDTIDWRDIVRDRDAEIVVKDNGRSCPPCHEVCKGRCWGPGSEDCQTLTKTICAPQCN |
| | P21860-3) | GHCFGPNPNQCCHDECAGGCSGPQDTDCFACRHFNDSGACVPRCPQPLVYNKLTFQLEPNPHTKYQYGG |
| | | VCVASCPHNFVVDQTSCVRACPPDKMEVDKNGLKMCEPCGGLCPKAF |
| 4 | Human HER3 | MGNLEIVLTGHNADLSFLQWIREVTGYVLVAMNEFSTLPLPNLRVVRGTQVYDGKFAIFVMLNYNTNSSHAL |
| | isoform 4 | RQLRLTQLTEILSGGVYIEKNDKLCHMDTIDWRDIVRDRDAEIVVKDNGRSCPPCHEVCKGRCWGPGSEDC |
| | (UniProt: | QTLTKTICAPQCNGHCFGPNPNQCCHDECAGGCSGPQDTDCFACRHFNDSGACVPRCPQPLVYNKLTFQL |
| | P21860-4) | EPNPHTKYQYGGVCVASCPHNFVVDQTSCVRACPPDKMEVDKNGLKMCEPCGGLCPKACEGTGSGSRFQ |
| | | TVDSSNIDGFVNCTKILGNLDFLITGLNGDPWHKIPALDPEKLNVFRTVREITGYLNIQSWPPHMHNFSVFSNL |
| | | TTIGGRSLYNRGFSLLIMKNLNVTSLGFRSLKEISAGRIYISANRQLCYHHSLNWTKVLRGPTEERLDIKHNRP |
| | | RRDCVAEGKVCDPLCSSGGCWGPGPGQCLSCRNYSRGGVCVTHCNFLNGEPREFAHEAECFSCHPECQ |
| | | PMEGTATCNGSGSDTCAQCAHFRDGPHCVSSCPHGVLGAKGPIYKYPDVQNECRPCHENCTQGCKGPEL |
| | | QDCLGQTLVLIGKTHLTMALTVIAGLVVIFMMLGGTFLYWRGRRIQNKRAMRRYLERGESIEPLDPSEKANKV |
| | | LARIFKETELRKLKVLGSGVFGTVHKGVWIPEGESIKIPVCIKVIEDKSGRQSFQAVTDHMLAIGSLDHAHIVRL |
| | | LGLCPGSSLQLVTQYLPLGSLLDHVRQHRGALGPQLLLNWGVQIAKGMYYLEEHGMVHRNLAARNVLLKSP |
| | | SQVQVADFGVADLLPPDDKQLLYSEAKTPIKWMALESIHFGKYTHQSDVWSYGVTVWELMTFGAEPYAGLR |
| | | LAEVPDLLEKGERLAQPQICTIDVYMVMVKCWMIDENIRPTFKELANEFTRMARDPPRYLVIKRESGPGIAPG |
| | | PEPHGLTNKKLEEVELEPELDLDLDLEAEEDNLATTTLGSALSLPVGTLNRPRGSQSLLSPSSGYMPMNQGN |
| | | LGESCQESAVSGSSERCPRPVSLHPMPRGCLASESSEGHVTGSEAELQEKVSMCRSRSRSRSPRPRGDS |
| | | AYHSQRHSLLTPVTPLSPPGLEEEDVNGYVMPDTHLKGTPSSREGTLSSVGLSSVLGTEEEDEDEEYEYMN |
| | | RRRRHSPPHPPRPSSLEELGYEYMDVGSDLSASLGSTQSCPLHPVPIMPTAGTTPDEDYEYMNRQRDGGG |
| | | PGGDYAAMGACPASEQGYEEMRAFQGPGHQAPHVHYARLKTLRSLEATDSAFDNPDYWHSRLFPKANAQ |
| | | RT |
| 5 | Human HER3 | MALTVIAGLVVIFMMLGGTFLYWRGRRIQNKRAMRRYLERGESIEPLDPSEKANKVLARIFKETELRKLKVLG |
| | isoform 5 | SGVFGTVHKGVWIPEGESIKIPVCIKVIEDKSGRQSFQAVTDHMLAIGSLDHAHIVRLLGLCPGSSLQLVTQYL |
| | (UniProt: | PLGSLLDHVRQHRGALGPQLLLNWGVQIAKGMYYLEEHGMVHRNLAARNVLLKSPSQVQVADFGVADLLP |
| | P21860-5) | PDDKQLLYSEAKTPIKWMALESIHFGKYTHQSDVWSYGVTVWELMTFGAEPYAGLRLAEVPDLLEKGERLA |
| | | QPQICTIDVYMVMVKCWMIDENIRPTFKELANEFTRMARDPPRYLVIKRESGPGIAPGPEPHGLTNKKLEEVE |
| | | LEPELDLDLDLEAEEDNLATTTLGSALSLPVGTLNRPRGSQSLLSPSSGYMPMNQGNLGESCQESAVSGSS |
| | | ERCPRPVSLHPMPRGCLASESSEGHVTGSEAELQEKVSMCRSRSRSRSPRPRGDSAYHSQRHSLLTPVTP |
| | | LSPPGLEEEDVNGYVMPDTHLKGTPSSREGTLSSVGLSSVLGTEEEDEDEEYEYMNRRRRHSPPHPPRPS |
| | | SLEELGYEYMDVGSDLSASLGSTQSCPLHPVPIMPTAGTTPDEDYEYMNRQRDGGGPGGDYAAMGACPA |
| | | SEQGYEEMRAFQGPGHQAPHVHYARLKTLRSLEATDSAFDNPDYWHSRLFPKANAQRT |
| 6 | Mature human | SEVGNSQAVCPGTLNGLSVTGDAENQYQTLYKLYERCEVVMGNLEIVLTGHNADLSFLQWIREVTGYVLVA |
| | HER3 isoform 1 | MNEFSTLPLPNLRVVRGTQVYDGKFAIFVMLNYNTNSSHALRQLRLTQLTEILSGGVYIEKNDKLCHMDTIDW |
| | (UniProt: | RDIVRDRDAEIVVKDNGRSCPPCHEVCKGRCWGPGSEDCQTLTKTICAPQCNGHCFGPNPNQCCHDECA |
| | P21860-1, v1 | GGCSGPQDTDCFACRHFNDSGACVPRCPQPLVYNKLTFQLEPNPHTKYQYGGVCVASCPHNFVVDQTSC |
| | positions 20 to | VRACPPDKMEVDKNGLKMCEPCGGLCPKACEGTGSGSRFQTVDSSNIDGFVNCTKILGNLDFLITGLNGDP |
| | 1342) | WHKIPALDPEKLNVFRTVREITGYLNIQSWPPHMHNFSVFSNLTTIGGRSLYNRGFSLLIMKNLNVTSLGFRS |
| | | LKEISAGRIYISANRQLCYHHSLNWTKVLRGPTEERLDIKHNRPRRDCVAEGKVCDPLCSSGGCWGPGPGQ |
| | | CLSCRNYSRGGVCVTHCNFLNGEPREFAHEAECFSCHPECQPMEGTATCNGSGSDTCAQCAHFRDGPHC |
| | | VSSCPHGVLGAKGPIYKYPDVQNECRPCHENCTQGCKGPELQDCLGQTLVLIGKTHLTMALTVIAGLVVIFM |
| | | MLGGTFLYWRGRRIQNKRAMRRYLERGESIEPLDPSEKANKVLARIFKETELRKLKVLGSGVFGTVHKGVWI |
| | | PEGESIKIPVCIKVIEDKSGRQSFQAVTDHMLAIGSLDHAHIVRLLGLCPGSSLQLVTQYLPLGSLLDHVRQHR |
| | | GALGPQLLLNWGVQIAKGMYYLEEHGMVHRNLAARNVLLKSPSQVQVADFGVADLLPPDDKQLLYSEAKTP |
| | | IKWMALESIHFGKYTHQSDVWSYGVTVWELMTFGAEPYAGLRLAEVPDLLEKGERLAQPQICTIDVYMVMV |
| | | KCWMIDENIRPTFKELANEFTRMARDPPRYLVIKRESGPGIAPGPEPHGLTNKKLEEVELEPELDLDLDLEAE |
| | | EDNLATTTLGSALSLPVGTLNRPRGSQSLLSPSSGYMPMNQGNLGESCQESAVSGSSERCPRPVSLHPMP |
| | | RGCLASESSEGHVTGSEAELQEKVSMCRSRSRSRSPRPRGDSAYHSQRHSLLTPVTPLSPPGLEEEDVNG |
| | | YVMPDTHLKGTPSSREGTLSSVGLSSVLGTEEEDEDEEYEYMNRRRRHSPPHPPRPSSLEELGYEYMDVG |
| | | SDLSASLGSTQSCPLHPVPIMPTAGTTPDEDYEYMNRQRDGGGPGGDYAAMGACPASEQGYEEMRAFQG |
| | | PGHQAPHVHYARLKTLRSLEATDSAFDNPDYWHSRLFPKANAQRT |
| 7 | Mature human | SEVGNSQAVCPGTLNGLSVTGDAENQYQTLYKLYERCEVVMGNLEIVLTGHNADLSFLQWIREVTGYVLVA |
| | HER3 isoform 2 | MNEFSTLPLPNLRVVRGTQVYDGKFAIFVMLNYNTNSSHALRQLRLTQLTGQFPMVPSGLTPQPAQDWYLL |
| | (UniProt: | DDDPRLLTLSASSKVPVTLAAV |
| | P21860-2 | |
| | positions 20 to | |
| | 183) | |
| 8 | Mature human | SEVGNSQAVCPGTLNGLSVTGDAENQYQTLYKLYERCEVVMGNLEIVLTGHNADLSFLQWIREVTGYVLVA |
| | HER3 isoform 3 | MNEFSTLPLPNLRVVRGTQVYDGKFAIFVMLNYNTNSSHALRQLRLTQLTEILSGGVYIEKNDKLCHMDTIDW |
| | (UniProt: | RDIVRDRDAEIVVKDNGRSCPPCHEVCKGRCWGPGSEDCQTLTKTICAPQCNGHCFGPNPNQCCHDECA |
| | P21860-3 | GGCSGPQDTDCFACRHFNDSGACVPRCPQPLVYNKLTFQLEPNPHTKYQYGGVCVASCPHNFVVDQTSC |
| | positions 20 to | VRACPPDKMEVDKNGLKMCEPCGGLCPKAF |
| | 331) | |
| 9 | Human HER3 | SEVGNSQAVCPGTLNGLSVTGDAENQYQTLYKLYERCEVVMGNLEIVLTGHNADLSFLQWIREVTGYVLVA |
| | isoform 1 | MNEFSTLPLPNLRVVRGTQVYDGKFAIFVMLNYNTNSSHALRQLRLTQLTEILSGGVYIEKNDKLCHMDTIDW |
| | extracellular | RDIVRDRDAEIVVKDNGRSCPPCHEVCKGRCWGPGSEDCQTLTKTICAPQCNGHCFGPNPNQCCHDECA |
| | region (UniProt: | GGCSGPQDTDCFACRHFNDSGACVPRCPQPLVYNKLTFQLEPNPHTKYQYGGVCVASCPHNFVVDQTSC |
| | P21860-1, v1 | VRACPPDKMEVDKNGLKMCEPCGGLCPKACEGTGSGSRFQTVDSSNIDGFVNCTKILGNLDFLITGLNGDP |
| | positions 20 to | WHKIPALDPEKLNVFRTVREITGYLNIQSWPPHMHNFSVFSNLTTIGGRSLYNRGFSLLIMKNLNVTSLGFRS |
| | 643) | LKEISAGRIYISANRQLCYHHSLNWTKVLRGPTEERLDIKHNRPRRDCVAEGKVCDPLCSSGGCWGPGPGQ |
| | | CLSCRNYSRGGVCVTHCNFLNGEPREFAHEAECFSCHPECQPMEGTATCNGSGSDTCAQCAHFRDGPHC |
| | | VSSCPHGVLGAKGPIYKYPDVQNECRPCHENCTQGCKGPELQDCLGQTLVLIGKTHLT |
| 10 | Human HER3 | MALTVIAGLVVIFMMLGGTFL |
| | isoform 1 | |
| | transmembrane | |
| | domain (UniProt: | |
| | P21860-1, v1 | |
| | positions 644 to | |
| | 664) | |
| 11 | Human HER3 | YWRGRRIQNKRAMRRYLERGESIEPLDPSEKANKVLARIFKETELRKLKVLGSGVFGTVHKGVWIPEGESIKI |
| | isoform 1 | PVCIKVIEDKSGRQSFQAVTDHMLAIGSLDHAHIVRLLGLCPGSSLQLVTQYLPLGSLLDHVRQHRGALGPQL |
| | cytoplasmic | LLNWGVQIAKGMYYLEEHGMVHRNLAARNVLLKSPSQVQVADFGVADLLPPDDKQLLYSEAKTPIKWMALE |
| | domain (UniProt: | SIHFGKYTHQSDVWSYGVTVWELMTFGAEPYAGLRLAEVPDLLEKGERLAQPQICTIDVYMVMVKCWMIDE |
| | P21860-1, v1 | NIRPTFKELANEFTRMARDPPRYLVIKRESGPGIAPGPEPHGLTNKKLEEVELEPELDLDLDLEAEEDNLATTT |
| | positions 665 to | LGSALSLPVGTLNRPRGSQSLLSPSSGYMPMNQGNLGESCQESAVSGSSERCPRPVSLHPMPRGCLASES |
| | 1342) | SEGHVTGSEAELQEKVSMCRSRSRSRSPRPRGDSAYHSQRHSLLTPVTPLSPPGLEEEDVNGYVMPDTHL |
| | | KGTPSSREGTLSSVGLSSVLGTEEEDEDEEYEYMNRRRRHSPPHPPRPSSLEELGYEYMDVGSDLSASLG |
| | | STQSCPLHPVPIMPTAGTTPDEDYEYMNRQRDGGGPGGDYAAMGACPASEQGYEEMRAFQGPGHQAPH |
| | | VHYARLKTLRSLEATDSAFDNPDYWHSRLFPKANAQRT |
| 12 | Human HER3 | YWRGRRIQNKRAMRRYLERGESIEPLDPSEKANKVLARIFKETE |
| | isoform 1 | |
| | juxtamembrane | |
| | segment | |
| | (UniProt: | |
| | P21860-1, v1 | |
| | positions 665 to | |
| | 708) | |
| 13 | Human HER3 | LRKLKVLGSGVFGTVHKGVWIPEGESIKIPVCIKVIEDKSGRQSFQAVTDHMLAIGSLDH |
| | isoform 1 protein | AHIVRLLGLCPGSSLQLVTQYLPLGSLLDHVRQHRGALGPQLLLNWGVQIAKGMYYLEEH |
| | kinase domain | GMVHRNLAARNVLLKSPSQVQVADFGVADLLPPDDKQLLYSEAKTPIKWMALESIHFGKY |
| | (UniProt: | THQSDVWSYGVTVWELMTFGAEPYAGLRLAEVPDLLEKGERLAQPQICTIDVYMVMVKCW |
| | P21860-1, v1 | MIDENIRPTFKELANEFT |
| | positions 709 to | |
| | 966) | |
| 14 | Human HER3 | RMARDPPRYLVIKRESGPGIAPGPEPHGLTNKKLEEVELEPELDLDLDLEAEEDNLATTTLGSALSLPVGTLN |
| | isoform 1 C | RPRGSQSLLSPSSGYMPMNQGNLGESCQESAVSGSSERCPRPVSLHPMPRGCLASESSEGHVTGSEAEL |
| | terminal | QEKVSMCRSRSRSRSPRPRGDSAYHSQRHSLLTPVTPLSPPGLEEEDVNGYVMPDTHLKGTPSSREGTLS |
| | segment | SVGLSSVLGTEEEDEDEEYEYMNRRRRHSPPHPPRPSSLEELGYEYMDVGSDLSASLGSTQSCPLHPVPIM |
| | (UniProt: | PTAGTTPDEDYEYMNRQRDGGGPGGDYAAMGACPASEQGYEEMRAFQGPGHQAPHVHYARLKTLRSLE |
| | P21860-1, v1 | ATDSAFDNPDYWHSRLFPKANAQRT |
| | positions 967 to | |
| | 1342) | |
| 15 | Human HER3 | SEVGNSQAVCPGTLNGLSVTGDAENQYQTLYKLYERCEVVMGNLEIVLTGHNADLSFLQWIREVTGYVLVA |
| | extracellular | MNEFSTLPLPNLRVVRGTQVYDGKFAIFVMLNYNTNSSHALRQLRLTQLTEILSGGVYIEKNDKLCHMDTIDW |
| | region | RDIVRDRDAEIVVKDNGRSC |
| | subdomain I | |
| | (UniProt: | |
| | P21860-1, v1 | |
| | positions 20 to | |
| | 183) | |
| 16 | Human HER3 | PPCHEVCKGRCWGPGSEDCQTLTKTICAPQCNGHCFGPNPNQCCHDECAGGCSGPQDTDCFACRHFND |
| | extracellular | SGACVPRCPQPLVYNKLTFQLEPNPHTKYQYGGVCVASCPHNFVVDQTSCVRACPPDKMEVDKNGLKMC |
| | region | EPCGGLCPK |
| | subdomain II | |
| | (UniProt: | |
| | P21860-1, v1 | |
| | positions 184 to | |
| | 329) | |
| 17 | Human HER3 | ACEGTGSGSRFQTVDSSNIDGFVNCTKILGNLDFLITGLNGDPWHKIPALDPEKLNVFRTVREITGYLNIQSW |
| | extracellular | PPHMHNFSVFSNLTTIGGRSLYNRGFSLLIMKNLNVTSLGFRSLKEISAGRIYISANRQLCYHHSLNWTKVLR |
| | region | GPTEERLDIKHNRPRRDCVA |
| | subdomain III | |
| | (UniProt: | |
| | P21860-1, v1 | |
| | positions 330 to | |
| | 495) | |
| 18 | Human HER3 | EGKVCDPLCSSGGCWGPGPGQCLSCRNYSRGGVCVTHCNFLNGEPREFAHEAECFSCHPECQPMEGTA |
| | extracellular | TCNGSGSDTCAQCAHFRDGPHCVSSCPHGVLGAKGPIYKYPDVQNECRPCHENCTQGCKGPELQDCLGQ |
| | region | TLVLIGKTHLT |
| | subdomain IV | |
| | (UniProt: | |
| | P21860-1, v1 | |
| | positions 496 to | |
| | 643) | |
| 19 | Human HER3 | QPLVYNKLTFQLEPNPH |
| | extracellular | |
| | region | |
| | subdomain II | |
| | dimerisation loop | |
| | (UniProt: | |
| | P21860-1, v1 | |
| | positions 261 to | |
| | 278) | |
| 20 | Rhesus | MGNLEIVLTGHNADLSFLQWIREVTGYVLVAMNEFSTLPLPNLRVVRGTQVYDGKFAIFVMLNYNTNSSHAL |
| | macaque HER3 | RQLRLTQLTEILSGGVYIEKNDKLCHMDTIDWKDIVRDQDAEIVVKDNGRSCPLCHEVCKGRCWGPGPEDC |
| | (UniProt: | QTLTKTICAPQCNGHCFGPNPNQCCHDECAGGCSGPQDTDCFACRHFNDSGACVPRCPQPLVYNKLTFQL |
| | F7HEH3-1, v2) | EPNPHTKYQYGGVCVASCPHNFVVDQTSCVRACPPDKMEVDKNGLKMCEPCGGLCPKACEGTGSGSRFQ |
| | | TVDSSNIDGFVNCTKILGNLDFLITGLNGDPWHKIPALDPEKLNVFRTVREITGYLNIQSWPPHMYNFSVFSNL |
| | | TTIGGRSLYNRGFSLLIMKNLNVTSLGFRSLKEISAGRIYISANRQLCYHHSLNWTKVLRGPTEERLDIKHNRP |
| | | RRDCVAEGKVCDPLCSSGGCWGPGPGQCLSCRNYSRGGVCVTHCNFLNGEPREFAHEAECFSCHPECQ |
| | | PMEGTATCNGSGSDTCAQCAHFRDGPHCVSSCPHGVLGAKGPIYKYPDVQNECRPCHENCTQGCKGPEL |
| | | QDCLGQTLVLIGKTHLTMALTVIAGLVVIFMMLGGTFLYWRGRRIQNKRAMRRYLERGESIEPLDPSEKANKV |
| | | LARIFKETELRKLKVLGSGVFGTVHKGVWIPEGESIKIPVCIKIIEDKSGRQSFQAVTDHMLAIGSLDHAHIVRLL |
| | | GLCPGSSLQLVTQYLPLGSLLDHVRQHRGALGPQLLLNWGVQIAKGMYYLEEHGMVHRNLAARNVLLKSPS |
| | | QVQVADFGVADLLPPDDKQLLYSEAKTPIKWMALESIHFGKYTHQSDVWSYGVTVWELMTFGAEPYAGLRL |
| | | AEVPDLLEKGERLAQPQICTIDVYMVMVKCWMIDENIRPTFKELANEFTRMARDPPRYLVIKRESGPGIAPGP |
| | | EPHGLTNKKLEEVELEPELDLDLDLEAEEDNLATTTLGSALSLPVGTLNRPRGSQSLLSPSSGYMPMNQGNL |
| | | GEAFQESAVSGSSEWCPRPVSLHPMPRGCLASESSEGHVTGSEAELQEKVSTCRSRSRSRSPRPRGDSA |
| | | YHSQRHSLLTPVTPLSPPGLEEEDVNGYVMPDTHLKGTPSSREGTLSSVGLSSVLGTEEEDEDEEYEYMNR |
| | | RRRHSPPRPPRPSSLEELGYEYMDVGSDLSASLGSTQSCPLHPVPVMPTAGTTPDEDYEYMNRQRGGSG |
| | | PGGDYAAMGACPASEQGYEEMRAFQGPGHQAPHVHYAHLKTLRSLEATDSAFDNPDYWHSRLFPKANAQ |
| | | RT |
| 21 | 10D1 heavy | DVQLQESGPDLVKPSQSLSLTCTVTGYSITSGYSWHWIRQFPGNKLEWMGSIHYSGGTNYNPSLKSRISITR |
| | chain variable | DTSKNQFFLQLNSVTTEDTATYFCARMTTAPRYPFDYWGQGTTLTVSS |
| | region | |
| 22 | 10D1_c75 heavy | DVQLQEWGAGLLKPSETLSLTCAVTGYSITSGYSWHWIRQFPGNGLEWIGSIHYSGGTNYNPTLKSRITISR |
| | chain variable | DTSKNQFSLKLSSVTAADTAVYFCARMTTAPRYPFDYWGQGTLVTVSS |
| | region | |
| 23 | 10D1_c76 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLKLSSVTAADTAVYFCARMTTAPRYPFDYWGQGTLVTVSS |
| | region | |
| 24 | 10D1_c77 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLKLSSVTAADTAVYFCARMTTAPRYPFDYWGQGTLVTVSS |
| | region | |
| 25 | 10D1_c78v1 | DVQLQEWGAGLLKPSETLSLTCAVTGYSITSGYSWHWIRQFPGNGLEWIGSIHYSGGTNYNPSLKSRITISR |
| | heavy chain | DTSKNQFSLKLSSVTAADTAVYFCARMTTAPRYPFDYWGQGTLVTVSS |
| | variable region | |
| 26 | 10D1_c78v2 | DVQLQEWGAGLLKPSETLSLTCAVTGYSITSGYSWHWIRQFPGKGLEWIGSIHYSGGTNYNPSLKSRITISRD |
| | heavy chain | TSKNQFSLKLSSVTAADTAVYFCARMTTAPRYPFDYWGQGTLVTVSS |
| | variable region | |
| 27 | 10D1_11B | DVQLQEWGAGLLKPSETLSLTCAVYGYSITSGYSWHWIRQPPGKGLEWIGSIHYSGGTNYNPSLKSRVTISR |
| | heavy chain | DTSKNQFSLKLSSVTAADTAVYYCARMTTAPRYPFDYWGQGTLVTVSS |
| | variable region | |
| 28 | 10D1_c85v1 | DVQLQEWGAGLLKPSETLSLTCAVTGYSITSGYSWHWIRQFPGNGLEWIGSIRYSGGTNYNPSLKSRITISR |
| | heavy chain | DTSKNQFSLKLGSVTAADTAVYFCARMTTAPRYPFDYWGQGTLVTVSS |
| | variable region | |
| 29 | 10D1_c85v2 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARMTTAPRYPFDYWGQGTLVTVSS |
| | variable region | |
| 30 | 10D1_c85o1 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARETTAPRYPFDYWGQGTLVTVSS |
| | variable region | |
| 31 | 10D1_c85o2 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARGTTAPRYPFDYWGQGTLVTVSS |
| | variable region | |
| 32 | 10D1_c87 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLRLSSVTAADTAVYFCARMTTAPRYPFDYWGQGTLVTVSS |
| | region | |
| 33 | 10D1_c89 heavy | QVQLQESGPGLVKPSQTLSLTCTVSGYSITSGYSWHWIRQHPGKGLEWIGSIRYSGGTDYNPSLKSLVTISA |
| | chain variable | DTSKNQFSLKLSSVTAADTAVYYCARMTTAPWYPFDYWGQGTTVTVSS |
| | region | |
| 34 | 10D1_c90 heavy | QVQLQESGPGLVKPSQTLFLTCTVSGYSITSGYSWHWIRQHPGKGLEWIGSIRYSGGTDYNPSLKSLVTISV |
| | chain variable | DTSKNQFSLKLSSVTAADTAVYYCARMTTAPWYPFDYWGQGTTVTVSS |
| | region | |
| 35 | 10D1_c91 heavy | QVQLQESGPGLVKPSQTLSLTCTVSGYYITSGYSWHWIRQHPGKGLEWIGSIRYSGGTDYNPSLKSLATISA |
| | chain variable | DTSKNQFSLKLSSVTAADTAVYYCARMTTAPWYPFDYWGQGTAVTVSS |
| | region | |
| 36 | 10D1_c92 heavy | DVQLQEWGAGLLKPSETLSLTCAVTGYSITSGYSWHWIRQFPGNGLEWIGSIHYSGGTNYNPTLKSRITISR |
| | chain variable | DTSKNQFSLKLSSVTAADTAVYFCARMTTAPRYPFDYWGQGTLVTVSS |
| | region | |
| 37 | 10D1_c93 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLRLSSVTAADTAVYFCARMTTAPRYPFDYWGQGTLVTVSS |
| | region | |
| 38 | 10D1, | GYSITSGYS |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c85v1, | |
| | 10D1_c85v2, | |
| | 10D1_c85o1, | |
| | 10D1_c85o2, | |
| | 10D1_c87, | |
| | 10D1_c89, | |
| | 10D1_c90, | |
| | 10D1_c92, | |
| | 10D1_c93 heavy | |
| | chain CDR1 | |
| 39 | 10D1_c91 heavy | GYYITSGYS |
| | chain CDR1 | |
| 40 | 10D1 derived | GYX₁ITSGYS |
| | consensus | wherein X₁ = S or Y |
| | heavy chain | |
| | CDR1 | |
| 41 | 10D1, | IHYSGGT |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c87, | |
| | 10D1_c92, | |
| | 10D1_c93 heavy | |
| | chain CDR2 | |
| 42 | 10D1_c85v1, | IRYSGGT |
| | 10D1_c85v2, | |
| | 10D1_c85o1, | |
| | 10D1_c85o2, | |
| | 10D1_c89, | |
| | 10D1_c90, | |
| | 10D1_c91 heavy | |
| | chain CDR2 | |
| 43 | 10D1 derived | IX₂YSGGT |
| | consensus | wherein X₂ = H or R |
| | heavy chain | |
| | CDR2 | |
| 44 | 10D1, | ARMTTAPRYPFDY |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c85v1, | |
| | 10D1_c85v2, | |
| | 10D1_c87, | |
| | 10D1_c92, | |
| | 10D1_c93 heavy | |
| | chain CDR3 | |
| 45 | 10D1_c89, | ARMTTAPWYPFDY |
| | 10D1_c90, | |
| | 10D1_c91 heavy | |
| | chain CDR3 | |
| 46 | 10D1_c85o1 | ARETTAPRYPFDY |
| | heavy chain | |
| | CDR3 | |
| 47 | 10D1_c85o2 | ARGTTAPRYPFDY |
| | heavy chain | |
| | CDR3 | |
| 48 | 10D1 derived | ARX₃TTAPX₄YPFDY |
| | consensus | wherein X₃ = M, E or G; X₄ = R or W |
| | heavy chain | |
| | CDR3 | |
| 49 | 10D1 light chain | DIVMTQSQKFMSTSVGDRVSVTCKASQIVGSNVAWYQQKPGQSPKPLIYSASYRYSGVPDRFTASGSGTDF |
| | variable region | TLTITNVQSEDLAEYFCQQYSSHPLTFGAGTKLELK |
| 50 | 10D1_c75 light | DIVMTQSPSSLSASVGDLVTITCKASQIVGSNVAWYQMKPGKSPKPLIYSASYLYFGVPSRFSGSGSGTDFT |
| | chain variable | LTISSLQPEDVAEYFCQQYSSHPLTFGPGTKVEIK |
| | region | |
| 51 | 10D1_c76 light | DIVMTQSPSSLSASGGDRVTITCKASQIVGYNVAWYQQKPGKSPKPLIYSASYLYSDVPSRFSASGSGTDFT |
| | chain variable | LTISSLQPEDVAEYFCQQYSSHPLTFGPGTKVEIK |
| | region | |
| 52 | 10D1_c77 light | VIVMTQSPSSLSASVGDRVTITCKASQIVGPNVAWYQQKPGKSPKPLIYSASYGYSDVPSRFSGSGSGTDFT |
| | chain variable | LTISSLQPEDVAEYFCQQYSTHPLTFGPGTKVEIK |
| | region | |
| 53 | 10D1_c78v1, | DIVMTQSPSSLSASVGDRVTITCKASQIVGSNVAWYQQKPGKSPKPLIYSASYGYSDVPSRFSGSGSGTDFT |
| | 10D1_c78v2, | LTISSLRPEDVATYYCQQYSSHPLTFGPGTKVEIK |
| | 10D1_11B light | |
| | chain variable | |
| | region | |
| 54 | 10D1_c85v1, | DIVMTQSPSSLSASVGDRVTITCKASQIVGSNVAWYQQKPGKSPKPLIYSARYQYSGVPFRFSGSGSGTDFT |
| | 10D1_c85v2 | LTISSLQPEDVATYYCQQYSSHPLTFGPGTKVEIK |
| | light chain | |
| | variable region | |
| 55 | 10D1_c85o1 | DIVMTQSPSSLSASVGDRVTITCKASQIVGSNVAWYQQKPGKSPKPLIYSARYQYSGVPFRFSGSGSGTDFT |
| | light chain | LTISSLQPEDVATYYCQQYSSHPLTFGPGTKVEIK |
| | variable region | |
| 56 | 10D1_c85o2 | DIVMTQSPSSLSASVGDRVTITCKASQIVGSNVAWYQQKPGKSPKPLIYSARYQYSGVPFRFSGSGSGTDFT |
| | light chain | LTISSLQPEDVATYYCQQYSSHPLTFGPGTKVEIK |
| | variable region | |
| 57 | 10D1_c87 light | DIVMTQSPSSLSASVGDRVTITCKASQIVGSNVAWYQQMPGKSPEPLIYSASYLYSDVPSRFSGSGSGTDFT |
| | chain variable | MTISSLQPEDVATYYCQQYSSHPLTFGPGTKVEIK |
| | region | |
| 58 | 10D1_c89 light | DIQMTQSPSSVSASVGDRVTITCKASQIVGSNVAWYQQKPGKAPEPLIYSASYLYSGVPSRFSGSGSGTDFT |
| | chain variable | LTISSLQPEDFATYYCQQYSSHPLTFGQGTKLEIK |
| | region | |
| 59 | 10D1_c90 light | DIQMTQSPSSVSASVGDRVTFTCKASQIVGSNVAWYQQKPGKAPEPLIYSASYLYSSVPSRFSGSGSGTEF |
| | chain variable | TMTISSLEPEDFATYYCQQYTTHPLTFGPGTKVEIK |
| | region | |
| 60 | 10D1_c91 light | DIQMTQSPSSVSASVGDRVTITCKASQIVGSNVAWYQQKPGKAPMPLIYSASYGYSGVPSRFSGSGSGTDF |
| | chain variable | TLTISSLQPEDFATYYCQQYSSHPLTFGQGTKLEIK |
| | region | |
| 61 | 10D1_c92 light | DIVMTQSPSSLSASVGDLVTITCKASQIVGSNVAWYQMKLGKSPKPLIYSASYLYFGVPSRFSGSGSGTDFTL |
| | chain variable | TISSLQPEDVAEYFCQQYFSHPLTFGPGTKVEIK |
| | region | |
| 62 | 10D1_c93 light | DIVMTQSPSSLSASVGDRVTITCKASQIVGSNVAWYQQKPGKSPKPLIYSASYLYSDVPSRFSGSGSGTDFT |
| | chain variable | MTISSLQPEDVATYYCQQYSSHPLTFGPGTKVEIK |
| | region | |
| 63 | 10D1, | QIVGSN |
| | 10D1_c75, | |
| | 10D1_c78v1, | |
| | 10D1_c78v2, | |
| | 10D1_11B, | |
| | 10D1_c85v1, | |
| | 10D1_c85v2, | |
| | 10D1_c85o1, | |
| | 10D1_c85o2, | |
| | 10D1_c87, | |
| | 10D1_c89, | |
| | 10D1_c90, | |
| | 10D1_c91, | |
| | 10D1_c92, | |
| | 10D1_c93 light | |
| | chain CDR1 | |
| 64 | 10D1_c76 light | QIVGYN |
| | chain CDR1 | |
| 65 | 10D1_c77 light | QIVGPN |
| | chain CDR1 | |
| 66 | 10D1 derived | QIVGX₅N |
| | consensus light | wherein X₅ = S, Y or P |
| | chain CDR1 | |
| 67 | 10D1, | SAS |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c87, | |
| | 10D1_c89, | |
| | 10D1_c90, | |
| | 10D1_c91, | |
| | 10D1_c92, | |
| | 10D1_c93 | |
| | light chain CDR2 | |
| 68 | 10D1_c85v1, | SAR |
| | 10D1_c85v2, | |
| | 10D1_c85o1, | |
| | 10D1_c85o2 | |
| | light chain CDR2 | |
| 69 | 10D1 derived | SAX₆ |
| | consensus light | wherein X₆ = S or R |
| | chain CDR2 | |
| 70 | 10D1, | QQYSSHPLT |
| | 10D1_c75, | |
| | 10D1_c76, | |
| | 10D1_c78v1, | |
| | 10D1_c78v2, | |
| | 10D1_11B, | |
| | 10D1_c85v1, | |
| | 10D1_c85v2, | |
| | 10D1_c85o1, | |
| | 10D1_c85o2, | |
| | 10D1_c87, | |
| | 10D1_c89, | |
| | 10D1_c91, | |
| | 10D1_c93 light | |
| | chain CDR3 | |
| 71 | 10D1_c77 light | QQYSTHPLT |
| | chain CDR3 | |
| 72 | 10D1_c90 light | QQYTTHPLT |
| | chain CDR3 | |
| 73 | 10D1_c92 light | QQYFSHPLT |
| | chain CDR3 | |
| 74 | 10D1 derived | QQYX₇X₈HPLT |
| | consensus light | wherein X₇ = S, T or F; X₈ = S or T |
| | chain CDR3 | |
| 75 | 10D1_c89 hIgG1 | QVQLQESGPGLVKPSQTLSLTCTVSGYSITSGYSWHWIRQHPGKGLEWIGSIRYSGGTDYNPSLKSLVTISA |
| | HC | DTSKNQFSLKLSSVTAADTAVYYCARMTTAPWYPFDYWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPEX₉X₁₀GGPSVFLFPPKPKDTLX₁₁IX₁₂RX₁₃PEVTCVVVDVSHEDPEVKFNWY |
| | | VDGVEVHNAKTKPREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| | | wherein X₉ = L or A; X₁₀ = L or A; X₁₁ = M or Y; X₁₂ = S or T; X₁₃ = T or E. |
| 76 | 10D1_c89 K LC | DIQMTQSPSSVSASVGDRVTITCKASQIVGSNVAWYQQKPGKAPEPLIYSASYLYSGVPSRFSGSGSGTDFT |
| | | LTISSLQPEDFATYYCQQYSSHPLTFGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYACEVTHQGLSSPVTKSFNRGEC |
| 77 | HER3 binding | CFGPNPNQCCHDECAGGC |
| | site for 10D1- | |
| | derived clones | |
| 78 | Binding site | PNPNQ |
| | motif 1 | |
| 79 | Binding site | DECAG |
| | motif 2 | |
| 80 | 10D1_c89 hIgG1 | QVQLQESGPGLVKPSQTLSLTCTVSGYSITSGYSWHWIRQHPGKGLEWIGSIRYSGGTDYNPSLKSLVTISA |
| | HC (mut1) | DTSKNQFSLKLSSVTAADTAVYYCARMTTAPWYPFDYWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPEAAGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGK |
| 81 | 10D1_c89 hIgG1 | QVQLQESGPGLVKPSQTLSLTCTVSGYSITSGYSWHWIRQHPGKGLEWIGSIRYSGGTDYNPSLKSLVTISA |
| | HC (mut2) | DTSKNQFSLKLSSVTAADTAVYYCARMTTAPWYPFDYWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLYITREPEVTCVVVDVSHEDPEVKFNWYVDGVEV |
| | | HNAKTKPREEQYNSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSRWQQGNVFSCSV |
| | | MHEALHNHYTQKSLSLSPGK |
| 82 | 10D1_c89 hIgG1 | QVQLQESGPGLVKPSQTLSLTCTVSGYSITSGYSWHWIRQHPGKGLEWIGSIRYSGGTDYNPSLKSLVTISA |
| | HC (mut1,2) | DTSKNQFSLKLSSVTAADTAVYYCARMTTAPWYPFDYWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPEAAGGPSVFLFPPKPKDTLYITREPEVTCVVVDVSHEDPEVKFNWYVDGVE |
| | | VHNAKTKPREEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGK |
| 83 | Human IgG1 | APELLGGPSVFLFPPKPKDTLMISRTPEVTCVVVDVSHEDPEVKFNWYVDGVEVHNAKTKPREEQYNSTYR |
| | CH2-CH3 region | VVSVLTVLHQDWLNGKEYKCKVSNKALPAPIEKTISKAKGQPREPQVYTLPPSRDELTKNQVSLTCLVKGFY |
| | (G1m1) | PSDIAVEWESNGQPENNYKTTPPVLDSDGSFFLYSKLTVDKSRWQQGNVFSCSVMHEALHNHYTQKSLSL |
| | | SPGK |
| 84 | Human IgG1 | APELLGGPSVFLFPPKPKDTLMISRTPEVTCVVVDVSHEDPEVKFNWYVDGVEVHNAKTKPREEQYNSTYR |
| | CH2-CH3 region | VVSVLTVLHQDWLNGKEYKCKVSNKALPAPIEKTISKAKGQPREPQVYTLPPSREEMTKNQVSLTCLVKGFY |
| | (G1m3) | PSDIAVEWESNGQPENNYKTTPPVLDSDGSFFLYSKLTVDKSRWQQGNVFSCSVMHEALHNHYTQKSLSL |
| | | SPGK |
| 85 | Human IgG2 | APPVAGPSVFLFPPKPKDTLMISRTPEVTCVVVDVSHEDPEVQFNWYVDGVEVHNAKTKPREEQFNSTFRV |
| | CH2-CH3 region | VSVLTWHQDWLNGKEYKCKVSNKGLPAPIEKTISKTKGQPREPQVYTLPPSREEMTKNQVSLTCLVKGFYP |
| | | SDISVEWESNGQPENNYKTTPPMLDSDGSFFLYSKLTVDKSRWQQGNVFSCSVMHEALHNHYTQKSLSLS |
| | | PGK |
| 86 | Human IgG3 | APELLGGPSVFLFPPKPKDTLMISRTPEVTCVVVDVSHEDPEVQFKWYVDGVEVHNAKTKPREEQYNSTFR |
| | CH2-CH3 region | VVSVLTVLHQDWLNGKEYKCKVSNKALPAPIEKTISKTKGQPREPQVYTLPPSREEMTKNQVSLTCLVKGFY |
| | | PSDIAVEWESSGQPENNYNTTPPMLDSDGSFFLYSKLTVDKSRWQQGNIFSCSVMHEALHNRFTQKSLSLS |
| | | PGK |
| 87 | Human IgG4 | APEFLGGPSVFLFPPKPKDTLMISRTPEVTCVVVDVSQEDPEVQFNWYVDGVEVHNAKTKPREEQFNSTYR |
| | CH2-CH3 region | VVSVLTVLHQDWLNGKEYKCKVSNKGLPSSIEKTISKAKGQPREPQVYTLPPSQEEMTKNQVSLTCLVKGFY |
| | | PSDIAVEWESNGQPENNYKTTPPVLDSDGSFFLYSRLTVDKSRWQEGNVFSCSVMHEALHNHYTQKSLSL |
| | | SLGK |
| 88 | Human IgG1 | APEAAGGPSVFLFPPKPKDTLMISRTPEVTCVVVDVSHEDPEVKFNWYVDGVEVHNAKTKPREEQYNSTYR |
| | CH2-CH3 region | VVSVLTVLHQDWLNGKEYKCKVSNKALPAPIEKTISKAKGQPREPQVYTLPPSRDELTKNQVSLTCLVKGFY |
| | (G1m3) mut1 | PSDIAVEWESNGQPENNYKTTPPVLDSDGSFFLYSKLTVDKSRWQQGNVFSCSVMHEALHNHYTQKSLSL |
| | | SPGK |
| 89 | Human IgG1 | APELLGGPSVFLFPPKPKDTLYITREPEVTCVVVDVSHEDPEVKFNWYVDGVEVHNAKTKPREEQYNSTYR |
| | CH2-CH3 region | VVSVLTVLHQDWLNGKEYKCKVSNKALPAPIEKTISKAKGQPREPQVYTLPPSRDELTKNQVSLTCLVKGFY |
| | (G1m3) mut2 | PSDIAVEWESNGQPENNYKTTPPVLDSDGSFFLYSKLTVDKSRWQQGNVFSCSVMHEALHNHYTQKSLSL |
| | | SPGK |
| 90 | Human IgG1 | APEAAGGPSVFLFPPKPKDTLYITREPEVTCVVVDVSHEDPEVKFNWYVDGVEVHNAKTKPREEQYNSTYR |
| | CH2-CH3 region | VVSVLTVLHQDWLNGKEYKCKVSNKALPAPIEKTISKAKGQPREPQVYTLPPSRDELTKNQVSLTCLVKGFY |
| | (G1m3) mut1,2 | PSDIAVEWESNGQPENNYKTTPPVLDSDGSFFLYSKLTVDKSRWQQGNVFSCSVMHEALHNHYTQKSLSL |
| | | SPGK |
| 91 | 10D1_c89 hIgG1 | QVQLQESGPGLVKPSQTLSLTCTVSGYSITSGYSWHWIRQHPGKGLEWIGSIRYSGGTDYNPSLKSLVTISA |
| | HC | DTSKNQFSLKLSSVTAADTAVYYCARMTTAPWYPFDYWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGK |
| 92 | Patritumab HC | QVQLQQWGAGLLKPSETLSLTCAVYGGSFSGYYWSWIRQPPGKGLEWIGEINHSGSTNYNPSLKSRVTISV |
| | | ETSKNQFSLKLSSVTAADTAVYYCARDKWTWYFDLWGRGTLVTVSSASTKGPSVFPLAPSSKSTSGGTAAL |
| | | GCLVKDYFPEPVTVSWNSGALTSGVHTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQPREPQVYTLPPSREE |
| | | MTKNQVSLTCLVKGFYPSDIAVEWESNGQPENNYKTTPPVLDSDGSFFLYSKLTVDKSRWQQGNVFSCSV |
| | | MHEALHNHYTQKSLSLSPGK |
| 93 | Patritumab LC | DIEMTQSPDSLAVSLGERATINCRSSQSVLYSSSNRNYLAWYQQNPGQPPKLLIYWASTRESGVPDRFSGS |
| | | GSGTDFTLTISSLQAEDVAVYYCQQYYSTPRTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPVTKSF |
| | | NRGEC |
| 94 | hIgG1 HC | ASTKGPSVFPLAPSSKSTSGGTAALGCLVKDYFPEPVTVSWNSGALTSGVHTFPAVLQSSGLYSLSSVVTVP |
| | (mut1) | SSSLGTQTYICNVNHKPSNTKVDKKVEPKSCDKTHTCPPCPAPEAAGGPSVFLFPPKPKDTLMISRTPEVTC |
| | | VVVDVSHEDPEVKFNWYVDGVEVHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 95 | hIgG1 HC | ASTKGPSVFPLAPSSKSTSGGTAALGCLVKDYFPEPVTVSWNSGALTSGVHTFPAVLQSSGLYSLSSVVTVP |
| | (mut2) | SSSLGTQTYICNVNHKPSNTKVDKKVEPKSCDKTHTCPPCPAPELLGGPSVFLFPPKPKDTLYITREPEVTCV |
| | | VVDVSHEDPEVKFNWYVDGVEVHNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 96 | hIgG1 HC | ASTKGPSVFPLAPSSKSTSGGTAALGCLVKDYFPEPVTVSWNSGALTSGVHTFPAVLQSSGLYSLSSVVTVP |
| | (mut1,2) | SSSLGTQTYICNVNHKPSNTKVDKKVEPKSCDKTHTCPPCPAPEAAGGPSVFLFPPKPKDTLYITREPEVTC |
| | | VVVDVSHEDPEVKFNWYVDGVEVHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 97 | ISO343c.S8D4 | DIEMTQSPDSLAVSLGERATINCRSSQSVLYSSSNRNYLAWYQQNPGQPPKLLIYWASTRESGVPDRFSGS |
| | LC | GSGTDFTLTISSLQAEDVAVYYCQQYYSTPRTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPVTKSF |
| | | NRGEC |
| 98 | ISO343c.S8D4 | QVQLQQWGAGLLKPSETLSLTCAVYGGSFSGYYWSWIRQPPGKGLEWIGEINHSGSTNYNPSLKSRVTISV |
| | HC | ETSKNQFSLKLSSVTAADTAVYYCARDKWTWYFDLWGRGTLVTVSSASTKGPSVFPLAPSSKSTSGGTAAL |
| | | GCLVKDYFPEPVTVSWNSGALTSGVHTFPAVLQSSGLYSLSSVVTVPSSSLGTQTYICNVNHKPSNTKVDKK |
| | | VEPKSCDKTHTCPPCPAPEAAGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRWQQGNVFSCSVM |
| | | HEALHNHYTQKSLSLSPGK |
| 99 | ISO209b.S8D4 | DIEMTQSPDSLAVSLGERATINCRSSQSVLYSSSNRNYLAWYQQNPGQPPKLLIYWASTRESGVPDRFSGS |
| | LC | GSGTDFTLTISSLQAEDVAVYYCQQYYSTPRTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPVTKSF |
| | | NRGEC |
| 100 | ISO209b.S8D4 | QVQLQQWGAGLLKPSETLSLTCAVYGGSFSGYYWSWIRQPPGKGLEWIGEINHSGSTNYNPSLKSRVTISV |
| | HC | ETSKNQFSLKLSSVTAADTAVYYCARDKWTWYFDLWGRGTLVTVSSASTKGPSVFPLAPSSKSTSGGTAAL |
| | | GCLVKDYFPEPVTVSWNSGALTSGVHTFPAVLQSSGLYSLSSVVTVPSSSLGTQTYICNVNHKPSNTKVDKK |
| | | VEPKSCDKTHTCPPCPAPEAAGGPSVFLFPPKPKDTLYITREPEVTCVVVDVSHEDPEVKFNWYVDGVEVH |
| | | NAKTKPREEQYNSTYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRWQQGNVFSCSVM |
| | | HEALHNHYTQKSLSLSPGK |

The present disclosure includes the combination of the aspects and preferred features described except where such a combination is clearly impermissible or expressly avoided.

The section headings used herein are for organizational purposes only and are not to be construed as limiting the subject-matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise’, and variations such as ‘comprises’ and ‘comprising’, will be understood to imply the inclusion of a stated integer or step or group of integers or steps but not the exclusion of any other integer or step or group of integers or steps.

It must be noted that, as used in the specification and the appended claims, the singular forms ‘a’, ‘an’, and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about’, it will be understood that the particular value forms another embodiment.

As used herein, a ‘peptide’ refers to a chain of two or more amino acid monomers linked by peptide bonds. A peptide typically has a length in the region of about 2 to 50 amino acids. A ‘polypeptide’ is a polymer chain of two or more peptides. Polypeptides typically have a length greater than about 50 amino acids.

Where a nucleic acid sequence is disclosed or referred to herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### EXAMPLES

### Example 1: Characterisation of 10D1F in WO 2019/185878 A1 and WO 2021/048274 A1

The HER3-binding antibody clone designated 10D1F is described in WO 2019/185878 A1 (incorporated by reference in its entirety). 10D1F comprises the heavy chain variable region shown in SEQ ID NO:36 of WO 2019/185878 A1 (=SEQ ID NO:33 of the present disclosure), and the light chain variable region shown in SEQ ID NO:83 of WO 2019/185878 A1 (=SEQ ID NO:58 of the present disclosure). 10D1F is also referred to in WO 2019/185878 A1 as ‘10D1_c89’.

Example 2.2 of WO 2019/185878 A1 describes a molecule (molecule [16]) comprising the VH and VL regions of 10D1F in human IgG1/Vκ format (10D1F hIgG1), formed of SEQ ID NO:206 of WO 2019/185878 A1 and SEQ ID NO:207 of WO 2019/185878 A1.

Examples 8.1 to 8.3 and FIGS. 42 to 46 of WO 2019/185878 A1 show that 10D1F hIgG1 binds to human HER3 with high affinity and specificity (displaying no cross-reactivity with other human EGFR family members), while retaining high-affinity binding to cyno, mouse and rat HER3.

Example 8.6 and FIGS. 49A and 49B of WO 2019/185878 A1 demonstrate that 10D1F hIgG1 binds to HER3 in a ligand (NRG)-independent fashion, and through a topologically distant epitope of HER3 to the epitope bound by anti-HER3 antibodies M-05-74 and M-08-11. Example 8.10 and FIG. 78 of WO 2021/048274 A1 demonstrate that 10D1F hIgG1 binds to human HER3 with subpicomolar affinity in the presence or absence of human NRG1.

WO 2021/048274 A1 at Example 3.5 also discloses that antibody clone 10D1 and 10D1-derived clones (including 10D1F) bind to human HER3 in the region corresponding to positions 218 to 235 of SEQ ID NO:1 (i.e. SEQ ID NO:77 of the present disclosure), and that within this region, two consensus binding site motifs were identified (shown in SEQ ID NOs:78 and 79 of the present disclosure).

Example 4.1 and FIG. 65, and Example 8.7 and FIG. 52 of WO 2019/185878 A1 demonstrate that 10D1F hIgG1 is highly potent at inhibiting interaction between HER3 and HER2, and does so in a dose-dependent manner. Example 8.7 and FIG. 53 of WO 2019/185878 A1 show that 10D1F hIgG1 inhibits interaction between HER3 and EGFR in a dose-dependent fashion.

Example 8.8 and FIG. 54 of WO 2019/185878 A1 show that 10D1F hIgG1 induces ADCC activity against HER3 overexpressing cells in a dose-dependent manner.

Example 8.9 and FIGS. 55, 63 and 64 of WO 2019/185878 A1 demonstrate that 10D1F hIgG1 inhibits HER3-mediated signalling in cells of HER3-expressing cancer cell lines in vitro.

Example 11 and FIG. 71 of WO 2019/185878 A1 show that 10D1F hIgG1 also inhibits HER3-mediated signalling in HER3-expressing human cancer cell line-derived xenograft tumors in vivo. Example 14 and FIG. 79 of WO 2021/048274 A1 demonstrate that 10D1F is extremely potent at inhibiting growth of xenograft tumors derived from a human cancer cell line harbouring an NRG gene fusion.

Examples 9.3, 9.4 and FIGS. 59, 60, 61, 62, 74 and 77 of WO 2019/185878 A1 demonstrate that 10D1F potently inhibits the growth of cancer cells in vitro, and also potently inhibits growth of human cancer cell line-derived xenograft tumors in vivo. Example 10 and FIGS. 67 and 68 of WO 2019/185878 A1 show that 10D1F hIgG1 inhibits in vitro proliferation of thyroid cancer cell lines harbouring the V600E BRAF mutation.

Example 13 and FIGS. 75 and 76 of WO 2019/185878 A1 demonstrate the utility of 10D1F hIgG1 to be employed for the detection of HER3.

Example 8.4 and FIG. 47A of WO 2019/185878 A1 show that 10D1F hIgG1 is thermostable, having a melting temperature of 70.0° C. as determined by Differential Scanning Fluorimetry.

Example 9.1, 9.2 and FIGS. 56, 57, 58 and 69, 70 of WO 2019/185878 A1 evidence that 10D1F hIgG1 has favourable pharmacological and toxicological profiles.

### Example 2: Production of Anti-HER3 Antibody-Drug Conjugates

### 2.1 Antibody Production

DNA sequences encoding the heavy and light chain variable regions of the anti-HER3 antibody clones were subcloned into mammalian multi-cistronic vectors pDZ1 or pTarget2.2, bearing human IgG1 Fc region mutations, or into expression vector pairs encoding the relevant antibody heavy and light chains. DNA sequence encoding the heavy and light chain variable regions of the anti-RSV antibody Palivizumab were subcloned into the mammalian dual-landing multi-cistronic vector sets pTargetF1314 and pTargetF3F bearing mutations in hIgG1 Fc region.

Antibodies were expressed using either (i) ExpiCHO-S Transient Expression System (Thermo Fisher, USA), (ii) CHO-k1 site-specific integration system, (iii) CHO-k1 random integration system, or (iv) CHO-k1 site-specific double landing pad integration system.

### ExpiCHO-S Transient Expression System

ExpiCHO-S cells were obtained from Thermo Fisher Scientific. Cells were cultured in serum-free, protein-free, animal origin-free, chemically defined medium with Glutamax supplementation (ExpiCHO Expression Medium, Thermo Fisher Scientific), at 37° C., in 8% CO₂ and 80% humidified incubators with shaking platform. Sub-culturing of cells was done when the cell reaches a density of 4 to 6×10⁶ cells/mL.

ExpiCHO-S cells were transfected with expression plasmids using ExpiFectamine transfection Reagent kit (Thermo Fisher Scientific) according to manufacturer's protocol. A day prior to transfection, ExpiCHO-S cells at maintenance (cell density of 4 to 6×10⁶ cells/mL) were seeded at 4×10⁶ cells/mL in ExpiCHO expression medium and allowed to grow overnight. On the day of transfection, cells reach 7 to 10×10⁶/ml viable cell density and viability of above 95%. Dilute cells to 6×10⁶ cells/mL of viable cells using fresh ExpiCHO expression medium for transfection. DNA-ExpiFectamine complexes were formed in serum-free medium, Optipro SFM (Thermo Fisher Scientific), for 1-5 min at room temperature before adding to the cells. ExpiFectamine CHO Enhancer and ExpiCHO feed (from ExpiFectamine Reagent Kit) were added to the transfected cells at 18-22 hours post transfection. A second volume of ExpiCHO feed was given to the cells on day 5 post-transfection and cells were placed at 32° C. From day 7 onwards, glucose was checked and topped up to 6 g/L.

Transfectants were harvested at day 14 by centrifugation at 4000×g for 10 min and filtered through 0.45 μm filter unit followed by 0.22 μm sterile filter units.

### CHO-k1 Site-Specific Integration System

CHO-k1 cells stably carrying site-specific recombinase recognition sequences FRT3 and FRT were maintained in commercially-available, serum-free, chemically-defined growth medium consisting of 50% HyQ PF CHO medium (Hyclone, USA), 50% CD CHO media (Gibco, USA) with 6 mM Gln, 0.05% Pluronic F68, and 600 μg/mL G418. The cells were passaged twice every week and cultured in 37° C., 8% CO₂ and 80% humidified shaker-incubator.

For transfections, 5 μg of circularised delivery plasmid pTarget2.2 bearing mAb sequences, along with 5 μg FLP recombinase expression plasmid, were co-transfected into 1×10⁷ CHO-k1 cells using 4D-Nucleofector kit (Lonza, Switzerland) with electroporation program FF-137. Electroporated cells were cultured at 37° C., 5% CO₂ humidified static cell incubator in wells of a 6-well plate containing 2 ml transfection medium (growth media without G418) for 12-24 hr. Medium was exchanged to fresh transfection medium 1 day after transfection by centrifugation, and the cultures were transferred to shaker-incubator. On day 5, medium was fully exchanged to selection medium (transfection medium with 20 μg/mL puromycin). Once viability improved, cells were passaged twice per week at a seeding density of 5×10⁵ cell/mL in selection medium, and eventually passaged at 3×10⁵ cells/mL when the viability was above 75%. Stable pools were established when the viability restored to 95% above. For mAb expression, established pool was cultured on a fed-batch cultivation mode, media feeds (Cytiva, USA), were dosed every alternative day from day 3 to day 11 at appropriate ratio. Glucose was checked daily and topped up to 6 g/L when below 6 g/L. The cultures were harvested on day 14-15. Cell culture supernatant contained the target mAb, and was clarified by acid flocculation or depth filtration.

### CHO-k1 Random Integration System

Host cell line CHO-k1 (CCL-61) was purchased from ATCC (US, lot #58995535). The cells were originally growing in adherent format and requires 10% bovine serum for proliferation. A 4-stage medium adaptation protocol was applied to adapt CHO-k1 cells to a serum-free and suspension cultivation format. Later, cells were adapted to a new commercially-available, serum-free, chemically-defined medium EX-CELL Advance CHO Fed-Batch Medium (Merck, USA) for further improving cell growth. Adapted cells were established as transfection host, and maintained in 37° C., 8% CO₂ and 80% humidified incubators with shaking platform.

For transfections, 1×10⁷ CHO-k1 cells were electroporated with 5 μg of linearized pDZ1 expression plasmid using 4D-Nucleofector kit (Lonza, Switzerland) with electroporation program CA201. Electroporated cells were maintained at 37° C., 5% CO₂ humidified static cell incubator in wells of a 6-well plate containing 2 ml growth medium, for 24 hr. Medium was exchanged to fresh selection medium by centrifugation was performed once per week starting from 24 hr post transfection in a seeding density 5×10⁵ cell/mL in static incubator. Cells were transferred to a shaker-incubator when viability restored and passaged twice per week with seeding density 5×10⁵ cell/mL. Stable pools were established by a series of selection medium exchanges in a static incubator, and a few passages in selection medium in a shaker-incubator. Established pools were subjected to a 14-15 day fed-batch cultivation process with intermediate medium feed, and glucose feeding to promote cell growth and productivity. Cell culture supernatant contained the target mAb, and was clarified by acid flocculation or depth filtration.

### CHO-k1 Site Specific Double Landing Pad Integration System

Host cell clone 2D5 originates from CHO-K1, and stably carries site-specific recombinase recognition sequences F3F and F13F14. The cells were maintained in commercially-available, serum-free, chemically-defined growth medium consisting of 50% HyQ PF CHO medium (Hyclone, USA), 50% CD CHO media (Gibco, USA) with 6 mM Gln, 0.05% Pluronic F68, 20 ug/mL Blasticidin and 600 μg/mL G418. The cells were passaged twice every week and cultured in 37° C., 8% CO₂ and 80% humidified shaker-incubator.

For transfections, 2.5 μg of circularised delivery plasmid ptargetF3F bearing with target mAb sequences, and 2.5 μg of circular delivery plasmid ptargetF13F14 bearing with target mAb sequences along with 5 μg FLP recombinase expression plasmid, were co-transfected into 1×10⁷ CHO-k1 2D5 cells using 4D-Nucleofector kit (Lonza, Switzerland) with electroporation program FF-137. Electroporated cells were maintained at 37° C., 5% CO₂ humidified static cell incubator in wells of a 6-well plate containing 2 ml transfection medium (growth media without Blasticidin and G418) for 12-24 hr. Medium was exchanged to fresh transfection medium 1 day after transfection by centrifugation, and the cultures were transferred to a shaker-incubator. On day 5, medium was fully exchanged to selection medium (transfection medium with 5 μg/mL puromycin+200 μg/mL Zeocin). Once viability improved, cells were passaged twice per week at a seeding density of 5×10⁵ cell/mL in selection medium, and eventually passaged at 3×10⁵ cells/mL when the viability was above 75%. Stable pools were established when the viability restored to 95% above. For mAb expression, established pools were cultured on a fed-batch cultivation mode, media feeds (Cytiva, USA), were dosed every alternative day from day 3 to day 11 at appropriate ratio. Glucose was checked daily and topped up to 6 g/L when below 6 g/L. The cultures were harvested on day 14-15. Cell culture supernatant contained the target mAb, and was clarified by acid flocculation or depth filtration.

### 2.2 Antibody Purification and Purity Analysis

Antibodies secreted into the culture supernatant from transfected cells were purified using liquid chromatography system AKTA Pure (Cytiva, USA). Specifically, supernatants were loaded onto mAbselect sure, or mAbselect sure LX, or mAbselect prismA columns (Cytiva, USA) at a contact time of 5-10 min, followed by washing the column with 3-5 column volumes of washing buffer (10 mM sodium phosphate+1 M NaCl, pH 7.2 and/or, 10 mM sodium phosphate+150 mM NaCl, pH 7.0 and/or 100 mM sodium citrate, pH 5.5). Bound mAbs were eluted with elution buffer (0.1 M sodium citrate, pH 3.5).

Where the aggregate/fragment level was higher than 8% as assessed by SE-HPLC, eluents were ‘polished’, as follows. Eluents were subjected to hold 1 hour at low pH for viral inactivation, followed by neutralization to pH 6.9. The neutralized pool was further diluted using 20 mM MES, pH 6.9 to reach conductivity 9.6 mS/cm, before being loaded onto polishing column Capto Adhere (Cytiva, USA). The mAbs were purified through flow-through method under the mobile phase of 20 mM MES, 80 mM NaCl, pH 6.9. Products were collected during sample application phase from 200 mAU A280 ascending peak to chasing phase 135 mAU A280 descending peak.

Eluents having an aggregate/fragment level ≤8% as assessed by SE-HPLC, and the polished antibody preparations were buffer exchanged into 20 mM histidine, 240 mM sucrose, 0.02% (w/w) polysorbate 80, pH 5.8, or PBS using 20K MWCO protein concentrators (Thermo Fisher, USA). Monoclonal antibody preparations were sterilized by passing through 0.1 μm filter, aliquoted and stored in −80° C. before use.

Antibody purity was analyzed by SE-HPLC using Acquity UPLC protein BEH SEC column 200, 1.7 um, 4.6 mm×150 mm column (Waters, USA) under mobile phase 250 mM NaCl, 100 mM Sodium phosphate, pH 6.8. on a Waters UPLC system (Waters, USA). 50 μg of antibody in 20 mM histidine, 240 mM sucrose, 0.02% (w/w) polysorbate 80, pH 5.8, or PBS, was injected to the column at a flow rate of 0.4 mL/min at room temperature. Proteins were eluted isocratically according to their molecular weights.

The SE-HPLC results for the different antibodies following purification (and prior to conjugation) are summarized below:

| | Antibody HC | |
|---|---|---|
| | and LC | Main Peak % by SE- |
| Molecule | Sequences | HPLC |
| 501c | SEQ ID NO: 80 | 94.58 |
| | SEQ ID NO: 76 | |
| 501b | SEQ ID NO: 82 | 98.95 |
| | SEQ ID NO: 76 | |
| ISO343c | SEQ ID NO: 97 | 97.89 |
| | SEQ ID NO: 98 | |
| ISO209b | SEQ ID NO: 99 | 96.56 |
| | SEQ ID NO: 100 | |

To further access the antibody purity, capillary electrophoresis sodium-dodecyl sulfate (CE-SDS) analysis was performed under non-reducing conditions, with a bare fused silica capillary of 50 μm ID×20 cm length to detector (Beckman Coulter, USA) using a PA800 system (Beckman Coulter, USA). Antibody samples in lowsalt buffer, or buffer exchanged into SDS-MW sample bufferwere diluted into 1 mg/mL in distilled water. 45 μL of diluted antibody was mixed with 57 μL master mix consisted of 5 μL IAM, 50 μL SDS-MW sample buffer, and 2 μL 10 kDa internal standard. The mixture was heated at 70° C. for 10 min then cooled to room temperature. Each sample was injected into the capillary for 20 seconds at 5 kV (reverse polarity), followed by separation at 15 kV (reverse polarity), for 30 mins in the capillary containing SDS-MW gel buffer. Data were analyzed using 32 Karat series software.

Product-related impurity host cell protein levels were analyzed using the CHO HCP ELISA kit (Cygnus, UK) following recommended protocol. Briefly, antibody samples or standard proteins were treated with alkaline phosphatase before coated onto microtiter strips which pre-coated with anti-CHO host cell proteins. The reaction was allowed to occur at room temperature for 2 hours followed by multiple washes. Subsequently, PNPP substrate was added and the reactions were held for 90 mins before reading on a plate reader with absorbance 405/492 nm.

The endotoxin levels in the purified antibody samples were analyzed using the Endosafe instrument (Charles River, USA) and Endosafe PTS Cartridges at sensitivity range 5-0.05 EU/mL (Charles River, USA), according to manufacturer's protocol.

### 2.3 Production of Antibody-Drup Conjugates

Antibody-SYNtecan E antibody-drug conjugates were prepared from the antibody preparations produced and purified as described in Examples 2.1 and 2.2, using GlycoConnect technology. GlycoConnect technology is described e.g. in van Geel et al., Bioconjug Chem. (2015) 26(11):2233-2242 and WO 2021/015622 A1, both of which are hereby incorporated by reference in their entirety.

The antibodies (see the table of Example 2.2 above) were dialyzed to remodelling buffer (20 mM histidine, 150 mM NaCl, 6 mM MnCl₂ pH 7.5), and incubated (at a concentration of 15 mg/ml) with endogylcosyltransferase SH (‘EndoSH’ described e.g. in WO 2017/137459 A1, at a concentration of 0.15 mg/ml), alkaline phosphatase (at a concentration of 0.0015 mg/ml), urine diphosphate (UDP)-6-azido-GalNAc (at a concentration of 1 mM) and Trichoplusia ni GalNAc transferase (TnGalNAcT, at a concentration of 0.45 mg/ml) for 16 hr at 30° C., to produce azido-modified antibodies in which the N-glycan of N297 of the CH2 domain of the Fc regions comprises a terminal 6-azido-GalNAc residue. Conversion to the azido-modified antibody was confirmed by mass spectral analysis (following treatment with IdeS, and detection of a ˜25 kDa Fc/2 fragment). The azido-modified antibodies were purified by protein A chromatography, and buffer-exchanged to TBS pH 7.4, at a concentration of ˜20 mg/ml.

Metal-free click conjugation of SYNtecan E was performed by incubating 10 mg/ml of the azido-modified antibody species (in TBS pH 7.4) with 4 equivalents (for the production of 501c.S8D4 and 501b.S8D4) or 5 equivalents (for the production of ISO343c.S8D4 and ISO209b.S8D4) of the following compound:

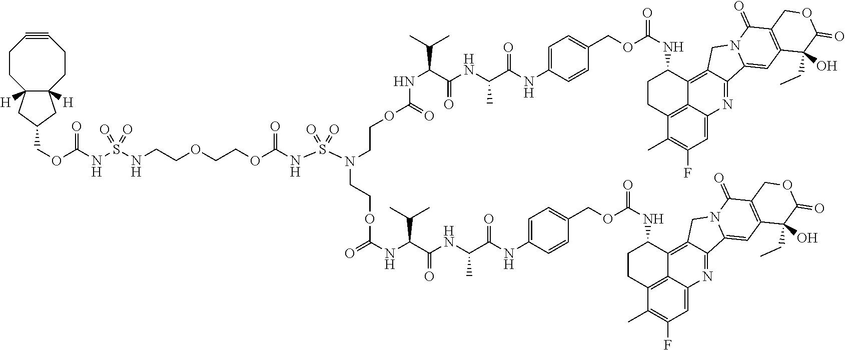 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)OCCN(CCOC(=O)N[C@H](C(=O)N[C@@H](C)C(=O)Nc2ccc(COC(=O)N[C@H]4CCc5c(C)c(F)cc6nc7c(c4c56)Cn4c-7cc5c(c4=O)COC(=O)[C@]5(O)CC)cc2)C(C)C)S(=O)(=O)NC(=O)OCCOCCNS(=O)(=O)NC(=O)OC[C@H]2C[C@H]4CCC#CCC[C@H]4C2)C(C)C)cc1)CC3]

in the presence of 30% propylene glycol, at room temperature, overnight. Conjugation reactions for the production of 501c.S8D4 and 501b.S8D4 additionally comprised 11 mM sodium deoxycholate.

Successful conjugation was evaluated by reverse-phase HPLC (RP-HPLC), following DTT reduction. Where the expected drug-to-antibody (DAR) ratio of 4 was not achieved, the samples were incubated with a further 1.5 equivalents of the compound for a further 5 hr.

The antibody-SYNtecan E conjugates were purified by size-exclusion chromatography using a Superdex 200 column, using PBS 7.4 as a mobile phase. Monomeric fractions were buffer-exchanged to buffer comprising 20 mM histidine, 6% sucrose, 0.04% tween 20, pH6.0 and filter-sterilized. Samples of the ADCs were then partitioned, snap-frozen and stored at −80° C.

The SE-HPLC results for the different antibody-drug conjugates following purification are summarized below:

| | Antibody HC | | |
|---|---|---|---|
| | and LC | | Main Peak % by |
| Molecule | Sequences | Linker-Payload | SE-HPLC |
| 501c.S8D4 | SEQ ID NO: 80 | SYNtecan E | >99 |
| | SEQ ID NO: 76 | | |
| 501b.S8D4 | SEQ ID NO: 82 | SYNtecan E | >97.3 |
| | SEQ ID NO: 76 | | |
| ISO343c.S8D4 | SEQ ID NO: 97 | SYNtecan E | >99 |
| | SEQ ID NO: 98 | | |
| ISO209b.S8D4 | SEQ ID NO: 99 | SYNtecan E | >99 |
| | SEQ ID NO: 100 | | |

### 2.4 Antibody-Drug Conjugates Characterized in Examples 3 and 4

The antibody-drug conjugates characterized in Examples 3 and 4 are shown in the table below:

| | Antibody HC | Linker- | | |
|---|---|---|---|---|
| | and LC | Payload | | |
| Molecule | Sequences | Name | Linker | Payload |
| 501c.S8D4 | SEQ ID NO: 80 | SYNtecan E | HydraSpace-PEG₂- | Exatecan |
| | SEQ ID NO: 76 | | HydraSpace-Val-Ala- | |
| | | | PABC | |
| 501b.S8D4 | SEQ ID NO: 82 | SYNtecan E | HydraSpace-PEG₂- | Exatecan |
| | SEQ ID NO: 76 | | HydraSpace-Val-Ala- | |
| | | | PABC | |
| Patritumab-DXd | SEQ ID NO: 92 | Deruxtecan | Maleimide-Gly-Gly-Phe- | DX-8951 |
| | SEQ ID NO: 93 | | Gly | |
| ISO343c.S8D4 | SEQ ID NO: 97 | SYNtecan E | HydraSpace-PEG₂- | Exatecan |
| | SEQ ID NO: 98 | | HydraSpace-Val-Ala- | |
| | | | PABC | |
| ISO209b.S8D4 | SEQ ID NO: 99 | SYNtecan E | HydraSpace-PEG₂- | Exatecan |
| | SEQ ID NO: 100 | | HydraSpace-Val-Ala- | |
| | | | PABC | |

A further control molecule comprising antibody-drug conjugate comprising an isotype-matched control antibody linked to deruxtecan was also employed in certain of the experiments (IgG-DXd).

### Example 3: In Vitro Characterisation of Anti-HER3 Antibody-Drug Conjugates

### 3.1 Binding to HER3 on the Surface of Different Cancer Cell Lines

Binding of the various antibody-drug conjugates (ADCs) described in Example 2 to different cell lines was evaluated by flow cytometry.

Binding to cells of the following cell lines was evaluated:

| Cell Line | Level of HER3 expression |
|---|---|
| HCT116 (CVCL_0291) | Low |
| DU145 (CVCL_0105) | Low |
| H358 (CVCL_1559) | Medium |
| T-47D (CVCL_0553) | High |
| OVCAR8 (CVCL_1629) | High |

Briefly, cells were seeded at 100,000 cells/well in wells of 96-well round bottom plates, and incubated (for 1 hr at 4° C.) with 8 different concentrations of the antibodies and ADCs, in a 3-fold dilution series (in PBS) starting from a highest concentration of 2 μg/ml.

Cells were subsequently washed twice with FACS buffer (PBS+5 mM EDTA and 0.5% BSA), and then incubated with F(ab′)2-Goat anti-Human IgG Fc Secondary Antibody, Alexa Fluor 488 (1:2000 dilution in FACS buffer) for 1 hr at 4° C.

Cells were subsequently washed twice with FACS buffer, and analysed by Flow Cytometry using Cytek Northern Light. Mean fluorescence intensities for the Alexa Fluor 488-positive cell populations were recorded, and EC₅₀ values for binding to cells of the different cell lines were derived from the binding curves.

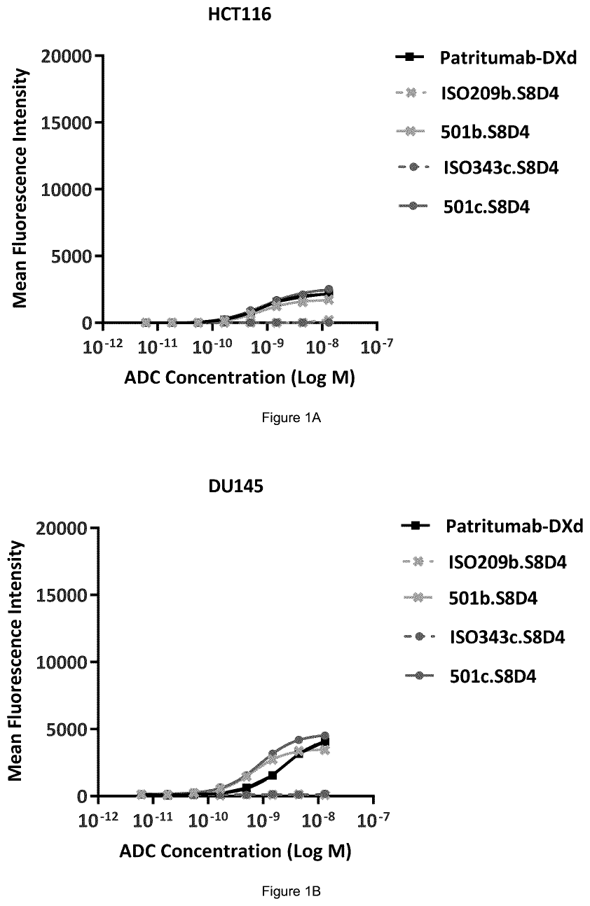

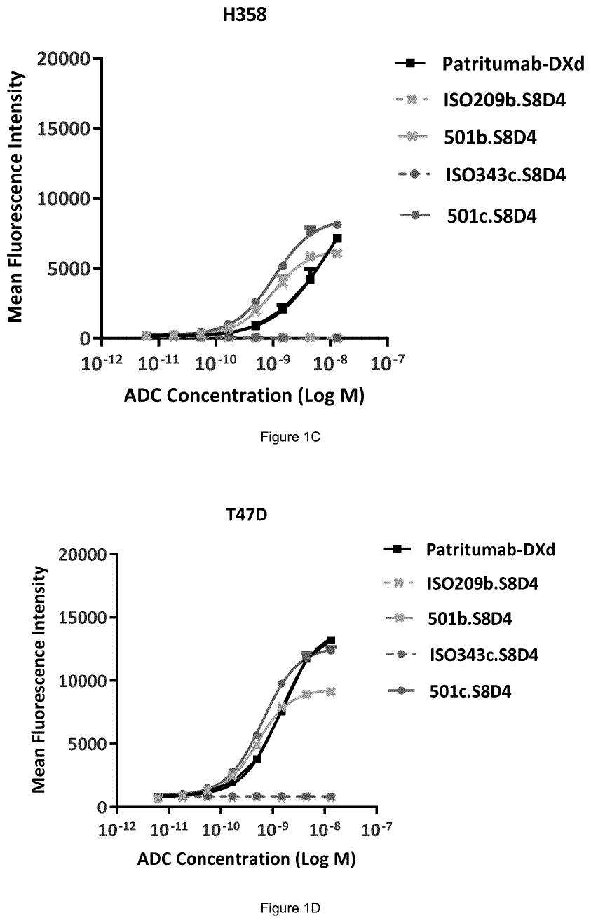

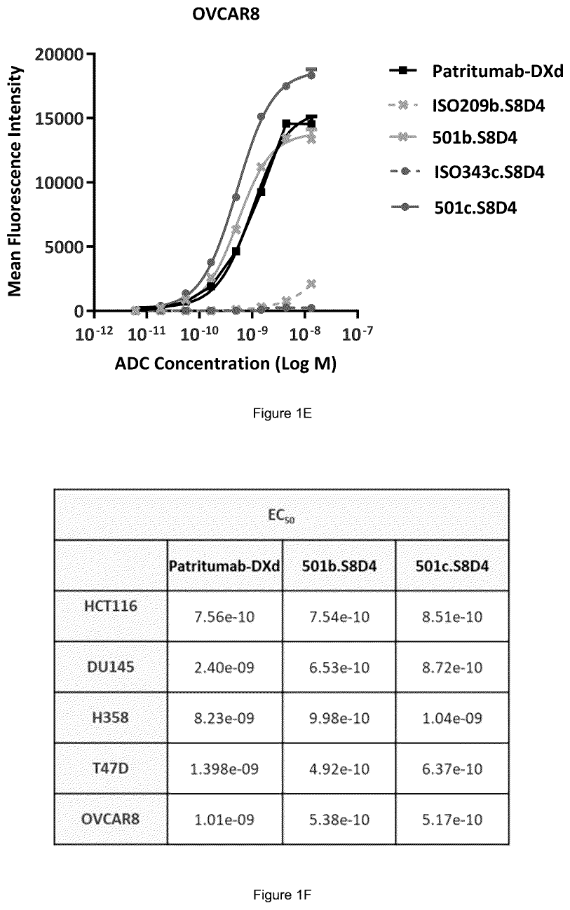

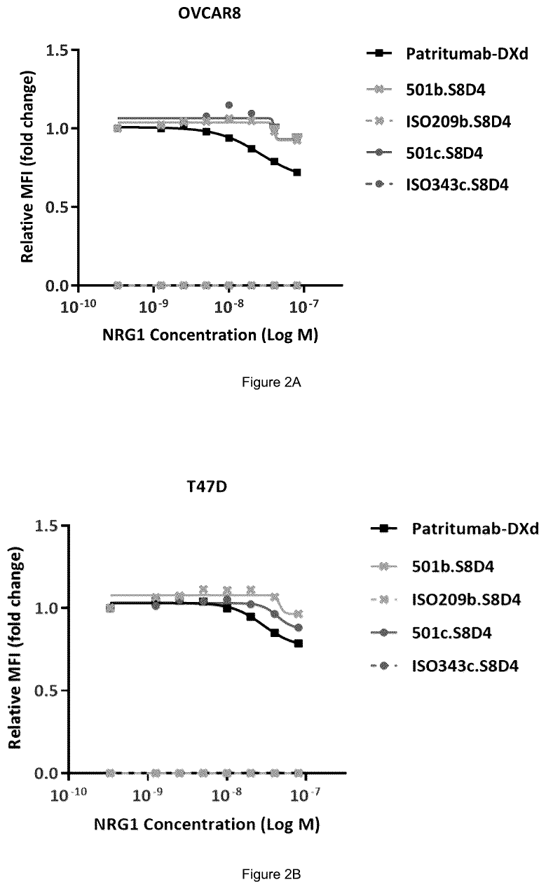

The results are shown in FIGS. 1A to 1F. ADCs comprising anti-HER3 antibodies displayed binding to all HER3-expressing cell lines tested. 501b.S8D4 and 501c.S8D4 displayed better cell surface binding to 4/5 cell lines tested as compared to Patritumab-DXd, as evidenced by their lower EC₅₀ values.

### 3.2 Binding to HER3 on the Surface of Different Cancer Cell Lines in the Presence of NRG1

Binding of ADCs described in Example 2 to various cell lines in the presence of the HER3 ligand NRG1 was evaluated by flow cytometry.

Briefly, cells were seeded at 100,000 cells/well in wells of 96-well round bottom plates, and incubated (for 3 hr at 4° C.) with 8 different concentrations of recombinant human NRG1, in a 2-fold dilution series (in PBS) starting from a highest concentration of 640 ng/ml, and in the presence of 2 μg/ml of the relevant antibody-drug conjugate.

Cells were subsequently washed twice with FACS buffer (PBS+5 mM EDTA and 0.5% BSA), and then incubated with F(ab′)2-Goat anti-Human IgG Fc Secondary Antibody, Alexa Fluor 488 (1:2000 dilution in FACS buffer) for 1 hr at 4° C.

Cells were subsequently washed twice with FACS buffer and analysed by Flow Cytometry using Cytek Northern Light. Mean fluorescence intensities for the Alexa Fluor 488-positive cell populations were recorded.

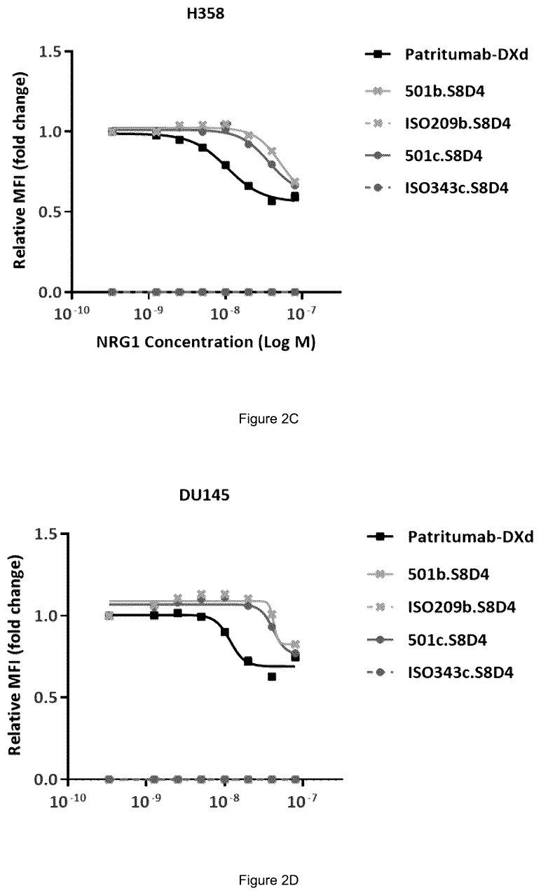

The results are shown in FIGS. 2A to 2D. 501c.S8D4 and 501 b.S8D4 were found to display significantly greater native binding to cells of the various HER3-expressing cell lines than Patritumab-DXd, in the presence of NRG1.

### 3.3 Binding to Recombinant Fcγ-Receptors

ADCs described in Example 2 were analysed for their ability to bind to human Fcγ receptors by surface plasmon resonance using Biacore.

Briefly, a Biacore HIS capture kit (Cytivia, Cat. #28995056) was employed to immobilize His-tagged recombinant human Fcγ receptor ligand (FcγRIA, FcγRIIA or FcγRIIIA) onto the surface of a CM5 sensor chip. 5 concentrations in a half-log dilution series of the various different analytes—Patritumab-DXd, 501b.S8D4, 501c.S8D4, a human IgG1 isotype-matched antibody (positive control) and a human IgG1 isotype-matched antibody comprising Fc-silencing mutations (as a negative control)—were applied to the chips, at a flow rate of 30 μl/min, for 60 sec association and 600 sec dissociation. Data capture and analysis were preformed using Biacore T200 according to manufacturer's recommendations for analyzing high-affinity antigen-antibody kinetics. Responses from all steps were reference cell-subtracted, and zero concentration-corrected to normalize for systematic disturbances. The resulting association and dissociation curves were fitted with 1:1 global fitting to obtain values for association rate (ka), dissociation rate (kd), and the equilibrium dissociation constant (KD).

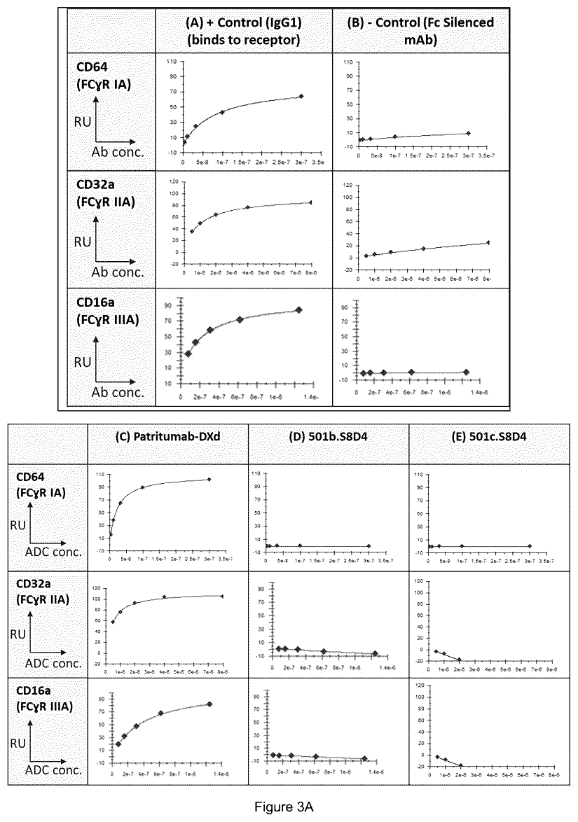

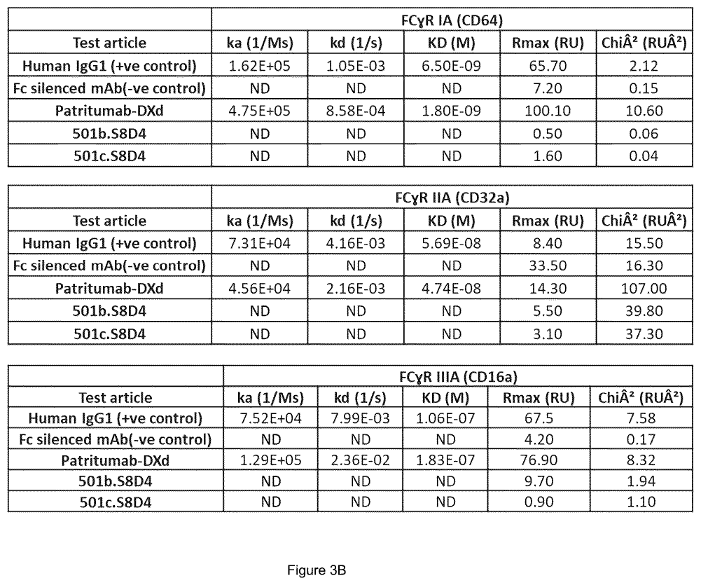

The results are shown in FIGS. 3A and 3B. 501c.S8D4 and 501b.S8D4 displayed complete abrogation of binding to FcγRIA, FcγRIIA and FcγRIIIA. Patritumab-DXd bound to all three FcγRs significantly with KD values comparable to the positive control.

Next, Fc-mediated binding of 501c.S8D4 to human PBMCs was evaluated compared with patritumab-DXd. Fc-mediated binding of ADCs to normal cells, including hematopoietic cells, could lead to severe thrombocytopenia, neutropenia, anemia and many other adverse events.

ADCs were labelled to a fluorochrome APC (red) using APC conjugation kit. PBMCs from 3 different donors were obtained. 100 ul of Zombie NIR™ Fixable Viability Dye (Biolegend, #423106) was added for every 1 million cells at 1:3,000 dilution using FACS buffer (0.5% BSA with 4 mM EDTA in PBS) and incubated at room temperature for 30 min. 100K cells/well were added to 96-well round bottom plates and incubated in the presence of 3 different concentrations (30, 10 and 3 μg/ml) of labelled 501c.S8D4, patritumab-DXd and IgG1-DXd isotype (positive) control for 1 h at 4° C. Post-incubation, the cells were washed, and the cell surface binding was quantified using flow cytometry using Cytek Northern Light 3000. Mean fluorescence intensity (MFI) was calculated and plotted using Graphpad Prism.

FIG. 16 shows that IgG-DXd shows the highest binding to PBMCs, because this isotype control does not bind to any known cell surface proteins in humans, whatever binding is observed is expected to be Fc-mediated (as with any IgG1 isotype). 501c.S8D4 showed significantly (81%) less binding compared to patritumab-DXd in PBMCs from all three donors.

In conclusion, 501c.S8D4 shows significantly reduced binding to human PBMCs, indicating reduced Fc-mediated uptake by healthy cells and reduced risk of associated toxicities.

### 3.4 Effect on Proliferation of HER3-Expressing Cells in a 3D In Vitro Proliferation Assay

The effect of various ADCs described in Example 2 on proliferation of T47D cells (CVCL_0553), H358 cells (CVCL_1559) and HCT116 cells (CVCL_0291) was evaluated in a 3D in vitro proliferation assay.

Briefly, cells were seeded at 2000 cells/well in RPMI media+10% FBS overnight at 37° C., 5% CO₂. After r 3 days, 100 ng/ml recombinant human NRG1 was added to some of the cultures, and cells were then treated with a 5-fold, 10-point dilution series of ADC/antibody starting from 50 μg/ml. Plates were then incubated at 37° C., 5% CO₂ for 7 days.

Cell viability was subsequently evaluated using the Cell Counting Kit-8 (Dojindo, #SKU-CK04), in accordance with the manufacturer's instructions. 20 μl of CCK8 reagent was added to each well, and plates read with a Biotek plate reader at 2 h, 4 h and 6 h post-addition of CCK8 reagent. Percentage inhibition of cell proliferation was calculated relative to the readings obtained in the presence of the lowest concentration of the drug or buffer only (i.e. in the absence of ADC/antibody). Data points show the mean of three replicates per condition. IC₅₀ values for inhibition of cell proliferation were derived from the binding curves.

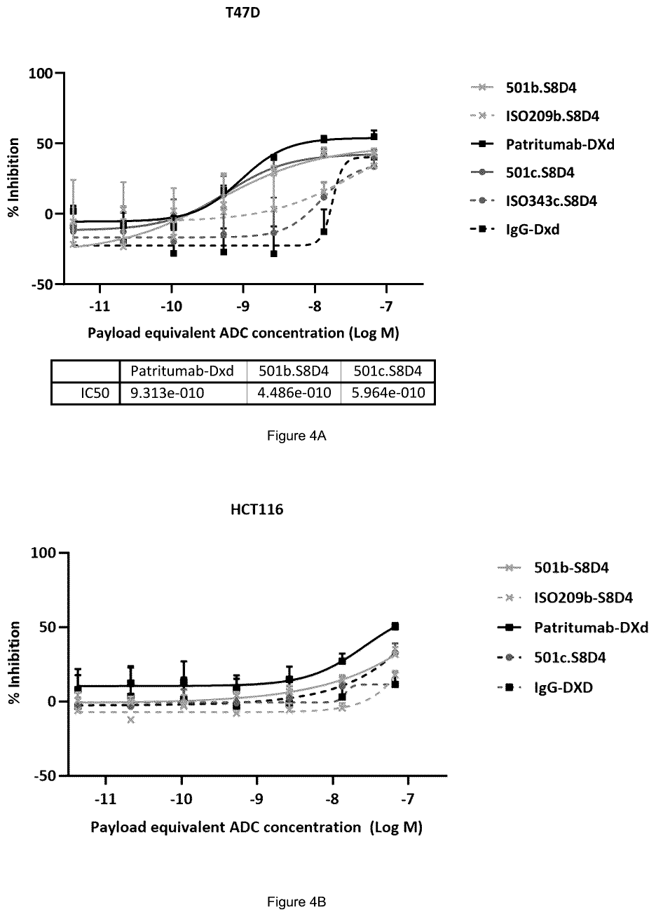

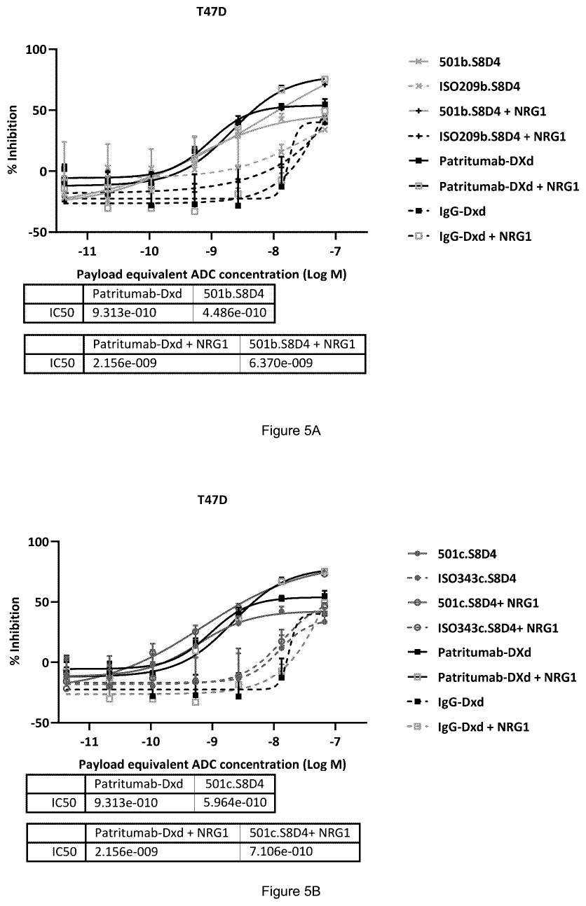

The results are shown in FIGS. 4A, 4B, 5A and 5B.

501c.S8D4 and 501b.S8D4 were found to inhibit proliferation of HER3-expressing T47D cells to a similar extent as Patritumab-DXd (FIG. 4A). None of the anti-HER3 antibody-drug conjugates substantially inhibited proliferation of HCT116 cells, which express low levels of HER3 (FIG. 4B).

In the absence of NRG1, 501b.S8D4 (IC₅₀=0.45 nM) and 501c.S8D4 (IC₅₀=0.59 nM) displayed a similar level of inhibition of cell proliferation, compared to Patritumab-DXd (0.93 nM; see FIGS. 5A and 5B)

In the presence of NRG1, 501c.S8D4 (IC₅₀=0.71 nM) displayed superior inhibition of cell proliferation compared to Patritumab-DXd (IC₅₀=2.2 nM; FIG. 5B), whereas 501b.S8D4 displayed a similar level of inhibition compared to Patritumab-DXd (FIG. 5A).

Taken together, these data indicate that 501c.S8D4 and 501b.S8D4 are effective in inhibiting proliferation of HER3-expressing cells in vitro, with comparable or improved effects relative to Patritumab-DXd.

### 3.5 Plasma Stability

The stability of 501c.S8D4 and 501b.S8D4 in plasma was investigated.

Briefly, 100 μl of 501c.S8D4 or 501b.S8D4 at a concentration of 100 μg/mL in mouse plasma were incubated for 0, 3, 7, 10, 14 and 21 days, at 37° C. Samples were collected at regular time points.

Antibodies were affinity-captured from the samples by addition of 4 μg of HIS-tagged HER3 (Sino Biological 10201-H08H) to 50 μl of each sample, followed by addition of 40 μl of anti-HIS tag mAb DPBS-washed, magnetic beads (MBL, D291-11). Samples were incubated with rotation for 2 hr at 4° C., and magnetic stands were used to isolate ADCs immobilised captured the beads. The beads were washed with 100 μl of ice-cold DPBS, and the captured ADCs were eluted with 50 μl of solution containing 30% acetonitrile, 1% formic acid in MilliQ water.

2 μl samples of the eluted ADCs were used for evaluation by liquid chromatography mass spectrometry (LCMS) in order to determine the percentage of the ADC detected that retains the correct antibody-to-drug ratio of 4.

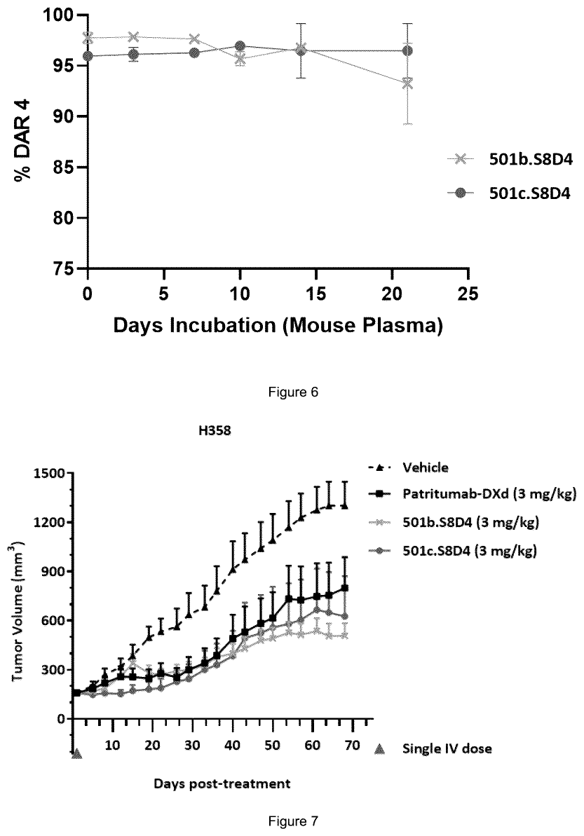

The results are shown in FIG. 6. 501c.S8D4 and 501b.S8D4 displayed good stability over the duration of the study, with a consistently high proportion of DAR4 species.

3.6 Internalization into HER3-Expressing Cells

501c.S8D4 was evaluated for its ability to be internalized and trafficked to the lysosomes in HER3-positive breast cancer cell line, T47D.

Fluorescence microscopy was performed on T47D cells (30,000 cells/well) treated with APC (red)-labelled 501c.S8D4 at 37° C. for 0.5, 4 and 16 h to study the localization of 501c.S8D4. One set of cells was incubated with APC-labelled 501c.S8D4 at 4° C. for 1 h as a negative control, as endocytosis does not occur at this temperature. Following incubation for the above-mentioned time points, the cells were washed, fixed and permeabilized. Next, the cells were stained with a FITC (green fluorescence)-labelled anti-LAMP1 antibody, which is a marker for lysosomes. Cells were imaged at 40× using Leica fluorescence microscope (DMi8) and processed using Thunder imager. In this assay, red fluorescence shows the localization of 501c.S8D4, green fluorescence shows localization of lysosomes, and a yellow color indicates co-localization of 501c.S8D4 and lysosomes.

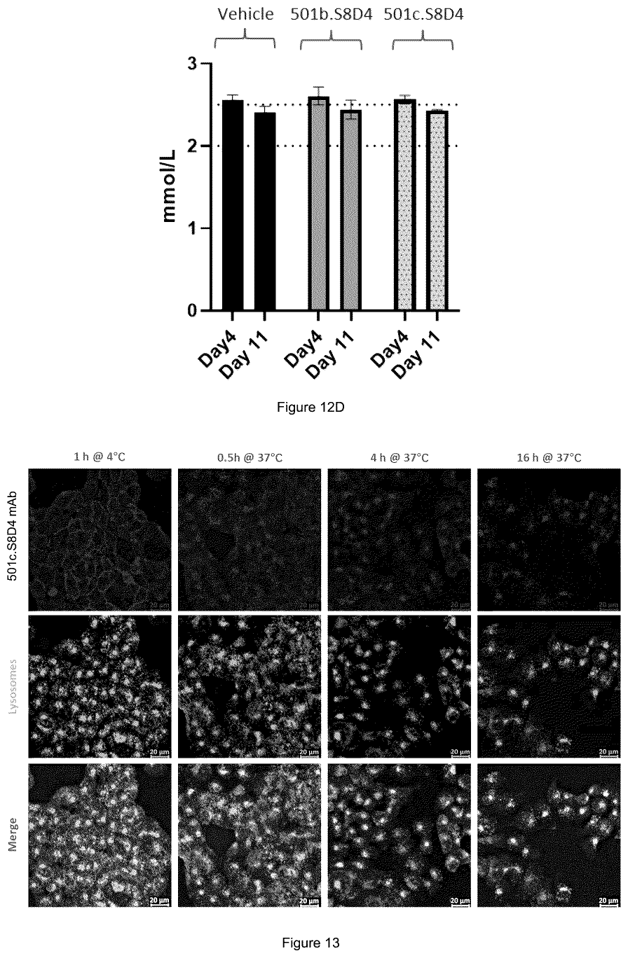

The results are shown in FIG. 13. The top panel of FIG. 13 shows that 501c.S8D4 is localized on the cell surface at 4° C., indicating binding to the cell surface HER3 without any observable internalization. Upon incubation at 37° C., 501c.S8D4 is rapidly internalized (within 30 mins) as shown by the punctate staining (indicating localization within endosomal compartments). In addition, an overlap analysis of the 501c.S8D4 (top) and lysosomal marker (middle) signals shows that 501c.S8D4 is trafficked into the lysosomes within 4 hours and stays in lysosomes for up to 16 hours in T47D cells as shown by co-localization signals in the merged (bottom) panels.

Thus, 501c.S8D4 is rapidly internalized upon incubation at 37° C. (within 30 mins) and is observed in the lysosomes for up to 16 hours.

The rate of internalization was then assessed. A pH-dependent fluorescent dye (Human Fabfluor-pH Red (Sartorius, #4722) was used to label 501c.S8D4. The dye fluoresces only at low pH (4.5-5.5), which is the pH within endocytic vesicles. Since the cell surface pH is around 7, the fluorescent signal can be detected only upon internalization of labelled 501c.S8D4 into the endocytic vesicles. The maximum signal comes when the dye reaches the lysosomes with the lowest pH of 4.5. The rate of internalization of an antibody or ADC was determined by measuring the fluorescent signal over time.

Internalization of 501c.S8D4 was measured using live cell imaging on T47D cells treated with Fab-fluor(red fluorescence)-labelled 501c.S8D4. 6000 T47D cells/well were seeded in a 96-well plate in RPMI media+10% FBS for 48 h at 37° C. in the presence of 5% CO₂. 12.5 nM of 501c.S8D4 was conjugated with Fabfluor for 15 mins at 37° C. at the ratio of 1:3 (ADC:Fabfluor). 100 μl of conjugated 501c.S8D4 was added to the cells. The plate was then incubated at 37° C. in the presence of 5% CO₂ for 2 days within the Incucyte® SX5 Live-Cell Analysis System. 4 images/well were captured every 30 mins during the incubation period. Images were analyzed using cell-by-cell analysis software (Incucyte) and mean red fluorescence intensity per cell was measured. Values were normalized to day 0 and data was plotted as percentage of red mean intensity using GraphPad PRISM.

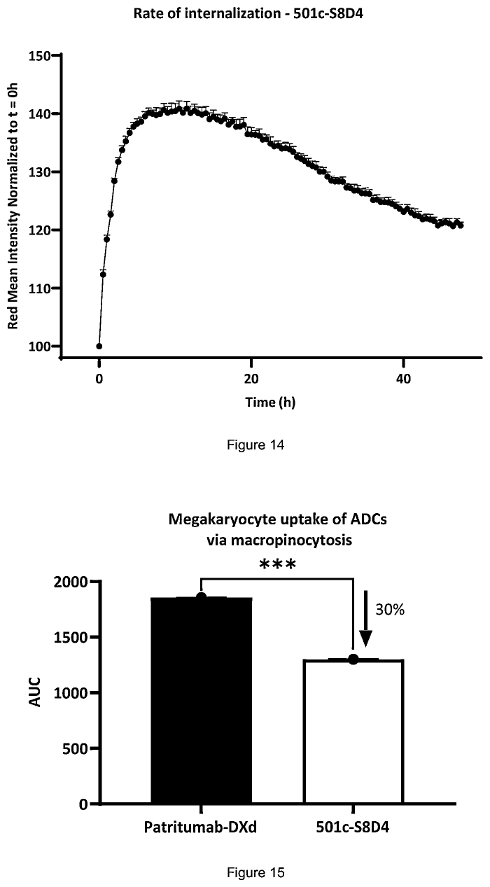

The results are shown in FIG. 14. 501c.S8D4 is internalized very quickly and reaches saturation levels within 12 h. The peak signal is observed when 501c.S8D4 has reached the lysosomes, and the signal starts to decrease due to lysosomal degradation of 501c.S8D4 after 24 h.

Thus, 501c.S8D4 is rapidly internalized and reaches saturation within 12 h in T47D cells.

### 3.7 ADC Macropinocytosis by Megakaryocytes

Non-target dependent uptake of 501c.S8D4 by human stem cell (HSC)-derived megakaryocytes (MKs) through macropinocytosis was evaluated. A pH-dependent fluorescent dye (Fab-Fluor, Red; Sartorius, #4722) was used to label 501c.S8D4 which fluoresces only at low pH (4.5-5.5), i.e. the pH within endocytic vesicles. Since the cell surface pH is around 7, the fluorescent signal can be detected only upon internalization of labelled 501c.S8D4 into the endocytic vesicles of MKs.

Thrombocytopenia, i.e., loss of platelets, is a very common side effect reported in cancer patients treated with ADCs, including patritumab-DXd which is a clinical stage HER3 ADC with similar class of payload. Platelets are produced by MKs that are derived from HSCs in bone marrows. Macropinocytosis-mediated uptake of ADCs by MKs prevents the production of platelets, eventually leading to thrombocytopenia. Thus, 501c.S8D4 was studied to determine whether macropinocytosis-mediated uptake happens with 501c. S8D4 treatment and how it compares with patritumab-DXd.

HSCs from three different donors were expanded and trans-differentiated to MKs. The MKs were then seeded in a 96 well plate (20000 cells/well) and incubated for 24 hours in SFEM II media (STEMcell technologies, #9655), StemSpan MK expansion supplement (STEMceII technologies, #2696), 10 ng/ml IL1 and 10 ng/ml IL3 at 37° C., with 5% CO₂. 100 μl of Fab-Fluor-labelled patritumab-DXd or 501c.S8D4 (ratio 1:3 of test article:Fab-Fluor) were added to the cells and the red fluorescence was measured on a real-time basis using live cell imaging for 24 hours, with 4 images/well captured every 30 mins. The mean red intensity was plotted against time after normalizing to time 0 (t=0). These values were plotted as percentage of red mean intensity of all three donors using GraphPad PRISM to obtain the internalization curves for 24 hours. The area under the curve is directly proportional to the total amount of ADCs internalized into megakaryocytes.

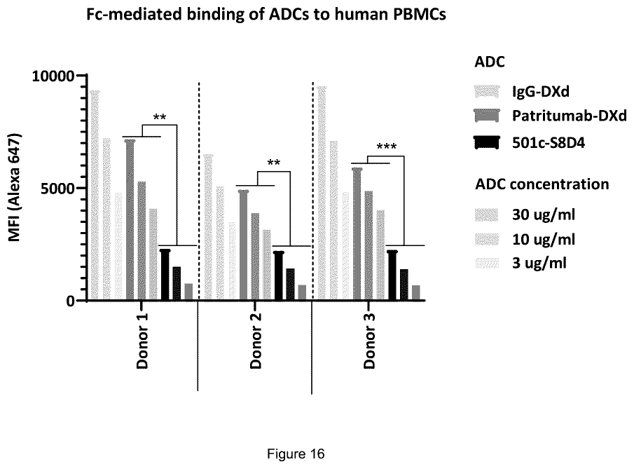

As shown in FIG. 15, macropinocytosis-dependent uptake of 501c.S8D4 by megakaryocytes is significantly (30%) less than the uptake of Patritumab-DXd.

These data indicate that the reduced uptake of 501c.S8D4 could potentially contribute to reduced thrombocytopenia in humans.

### Example 4: In Vivo Characterisation of Anti-HER3 Antibody-Drug Conjugates

### 4.1 Anti-Tumor Efficacy In Vivo

The therapeutic efficacy of various ADCs described in Example 2 was investigated in vivo, in a human cancer cell line-derived xenograft model of lung adenocarcinoma, H358.

Approximately 6-8 week old female Ncr Nude mice were housed under specific pathogen-free conditions and treated in compliance with the Institutional Animal Care and Use Committee (IACUC) guidelines. H358 (CVCL_1559) cell-derived tumors were established by admixture of 5×10⁶ cells with equal volume of Matrigel (Corning, USA), and subcutaneous implantation into the right flanks of mice. Treatment was initiated when tumours reached approximately 150 to 160 mm³. Mice were intravenously administered a single, 3 mg/kg bodyweight dose of the relevant antigen-binding molecule at Day 1. There were 7 mice per treatment group.

Tumor volume was measured 3 times a week using a digital Vernier Caliper and calculated using the formula [L×W2/2]. Study end point was reached once the tumors of the control arm reached the volume of >1.5 cm³.

The results of the experiments are shown in FIG. 7. 501c.S8D4 and 501b.S8D4 displayed significant inhibition of growth of H358 CDX tumors (which express HER3 at a medium level of expression). 501b.S8D4 displayed a similar level of tumor growth inhibition as Patritumab-DXd, and 501c.S8D4 displayed an improved level of tumor growth inhibition as compared to Patritumab-DXd. Thus, despite having a lower drug-to-antibody ratio (DAR) of 4 relative to Patritumab-DXd, which has a DAR of 8, 501c.S8D4 and 501b.S8D4 displayed similar or improved tumor growth inhibition as compared to Patritumab-DXd. These data indicate that 501c.S8D4 and 501b.S8D4 have superior efficacy than Patritumab-DXd in this H358 CDX model. In addition, the tumor suppression displayed by 501c.S8D4 and 501b.S8D4 was much more prolonged than for Patritumab-DXd, suggesting that 501c.S8D4 and 501 b.S8D4 might be more stable than Patritumab-DXd in vivo.

In another in vivo experiment, T47D cells (CVCL 0553) cell-derived tumors were established in 6-8 week old female NOD/SCID mice by admixture of 1×10⁷ cells with equal volume of Matrigel (Corning, USA), and subcutaneous implantation into the right flanks of mice. As these tumor cells are dependent on estrogen for their growth, slow release (60 days) estrogen pellets were implanted one day prior to implantation of the tumor (Ruan et al., Maturitas. (2019) 123:1-8). Treatment was initiated when tumours reached approximately 150 to 250 mm³. There were 7 mice per treatment group. Mice were intravenously administered a single dose of the relevant antigen-binding molecule at Day 1, as follows:
Arm 1: Vehicle only (PBS)
Arm 2: Patritumab-DXd, 1.5 mg/kg bodyweight
Arm 3: Patritumab-DXd, 4.5 mg/kg bodyweight
Arm 4: 501b.S8D4, 3 mg/kg bodyweight
Arm 5: 501b.S8D4, 9 mg/kg bodyweight
Arm 6: 501c.S8D4, 3 mg/kg bodyweight
Arm 7: 501c.S8D4, 9 mg/kg bodyweight

Tumor volume was measured 3 times a week as described above.

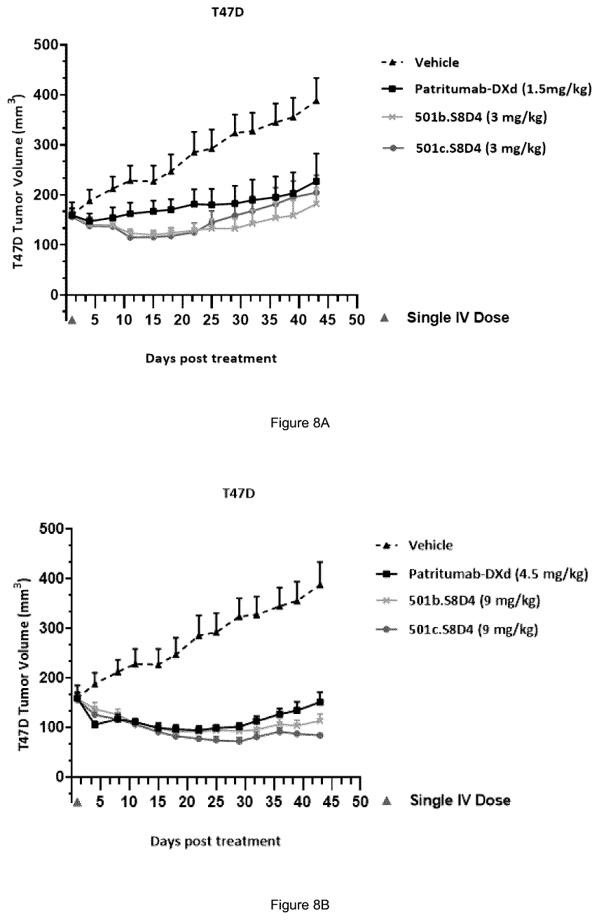

The results are shown in FIGS. 8A and 8B. 3 mg/kg bodyweight 501c.S8D4 and 501b.S8D4 showed significantly higher tumor growth inhibition as compared to Patritumab-DXd at equivalent payload dose (i.e. 1.5 mg/kg bodyweight Patritumab-DXd).

9 mg/kg bodyweight 501c.S8D4 and 501b.S8D4 displayed similar tumor growth inhibition to Patritumab-DXd at equivalent payload dose (i.e. 4.5 mg/kg bodyweight Patritumab-DXd).

Taken together, these data suggest that 501c.S8D4 and 501b.S8D4 possess superior efficacy in vivo in the T47D cell line-derived model.

### 4.2 Analysis of Toxicology

Toxicities including severe interstitial lung disease (ILD) and high grade cytopenia have been observed following administration of Patritumab-DXd (Jsnne et al., Cancer Discov. (2022) 12(1):74-89). These toxicities might arise as a consequence of Fcγ receptor- and/or Macropinocytosis-mediated uptake of ADCs by normal, non-cancerous cells (Zhao et al., Mol Cancer Ther (2017) 16(9): 1866-1876; Kumagai et al., Cancer Sci. (2020) 111(12): 4636-4645; Zhao et al., Mol Cancer Ther (2017) 16(9): 1877-1886).

The toxicity of various ADCs described in Example 2 was evaluated in female Sprague Dawley rats. The treatment groups were as follows:
Group 1: Vehicle only
Group 2: 501b.S8D4
Group 3: 501c.S8D4

ADCs or an equal volume of vehicle was administered by slow intravenous injection into the tail vein. A first dose of 60 mg/kg bodyweight was administered on Day 1, and a second dose of 53 mg/kg bodyweight was administered on Day 8. There were 3 mice per treatment group.

Survival, bodyweight, and food consumption were monitored. Blood samples were collected for analysis on Days 4 and 11, and full necropsies were performed on Day 11.

The health of the treated rats, including, clinical signs, body weight, food consumption and mortality was monitored every day posttreatment and recorded. Tissues including liver, kidney, lung, brain, bone marrow, skin, stomach, small and large intestines, any tissue with gross lesions were collected and processed to perform histopathological analysis for tissue damage.

All mice in each treatment group survived for the full 11 days of the experiment. There was no significant loss of body weight or change in food consumption observed throughout the treatment period.

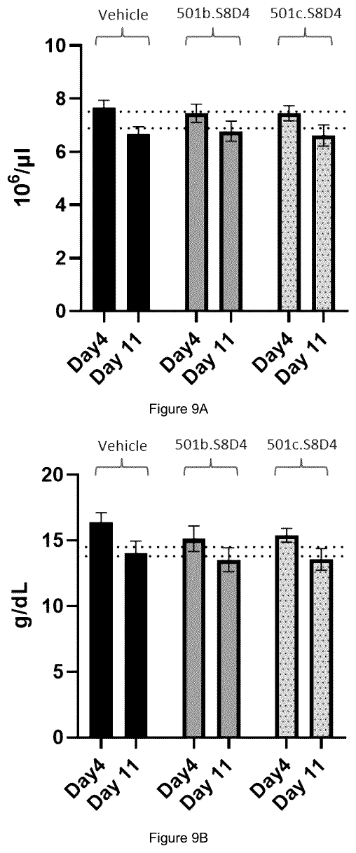

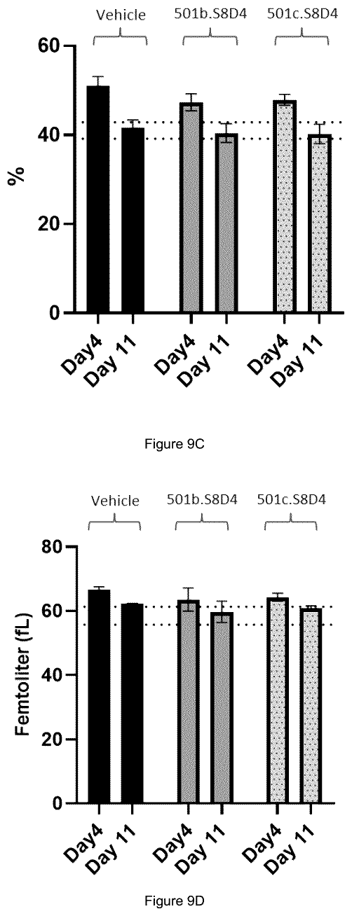

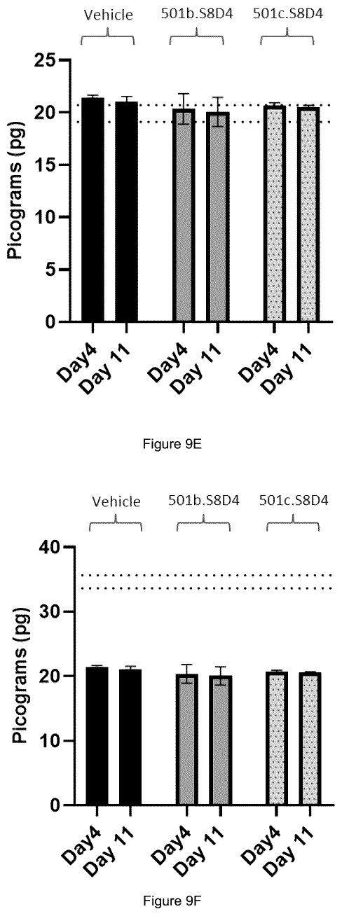

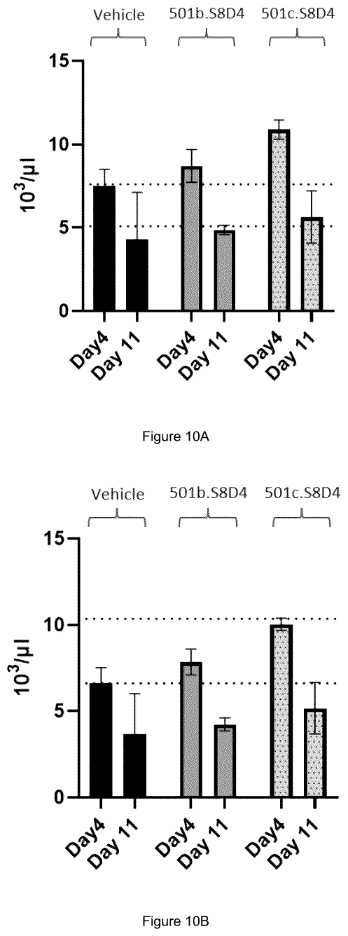

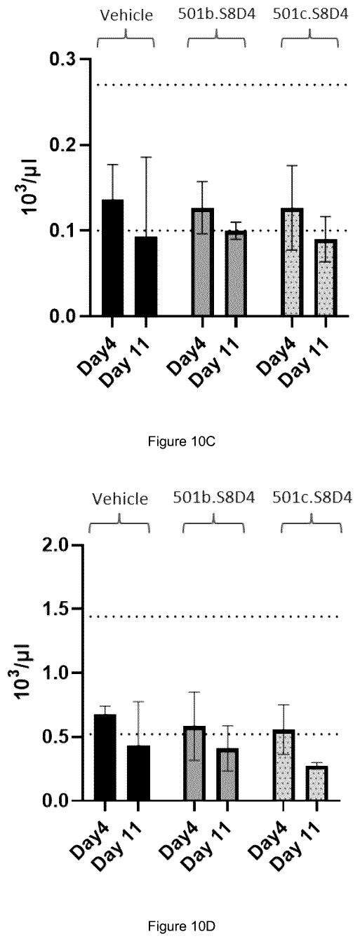

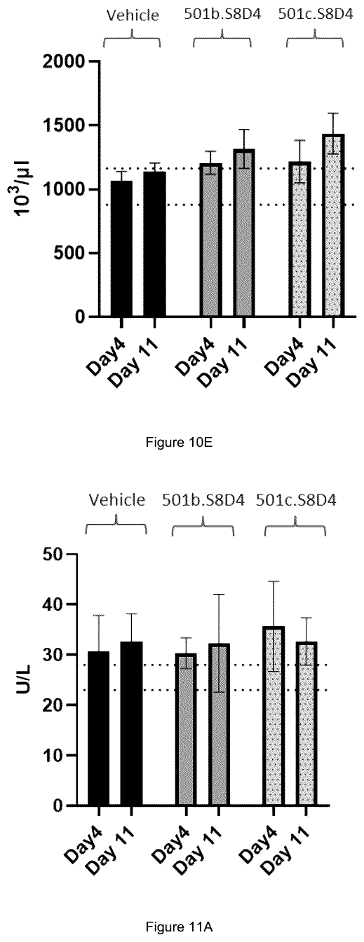

Red blood cell counts, hemoglobin concentration, hematocrit percentage, mean corpuscular volume (MCV), mean corpuscular hemoglobin (MCH) level, and mean corpuscular hemoglobin concentration (MCHC) were found not to differ significantly between blood samples obtained from rats in Groups 1, 2 and 3 (FIGS. 9A to 9F). Rats in Groups 2 and 3 did not display any significant signs of neutropenia (FIG. 10D) or thrombocytopenia (FIG. 10E), which have previously been reported in clinical studies of Patritumab-DXd (Steuer et al., Journal of Clinical Oncology (2022) 40 (16_suppl) 9017; Janne et al., Cancer Discov (2022)12(1):74-89)—see FIGS. 10A to 10E. A significant reduction in white blood cell counts has previously been reported in preclinical studies of Patritumab-DXd in rats (Hashimoto et al., Clin Cancer Res. (2019)25(23):7151-7161), however in the present study an increase in white blood cell counts was observed in Groups 2 and 3 relative to Group 1 (FIG. 10A).

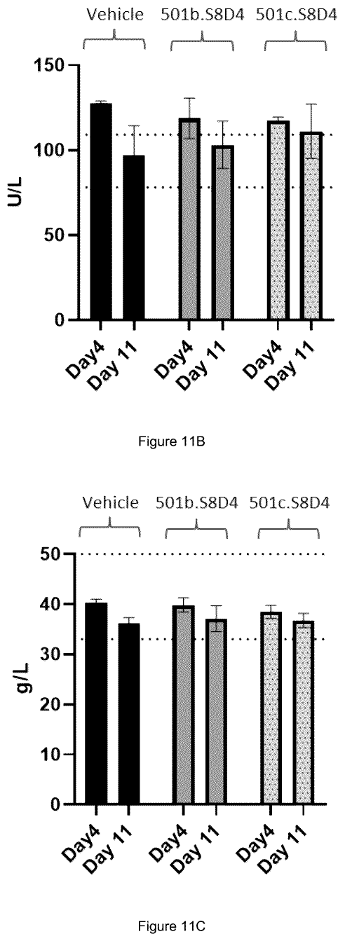

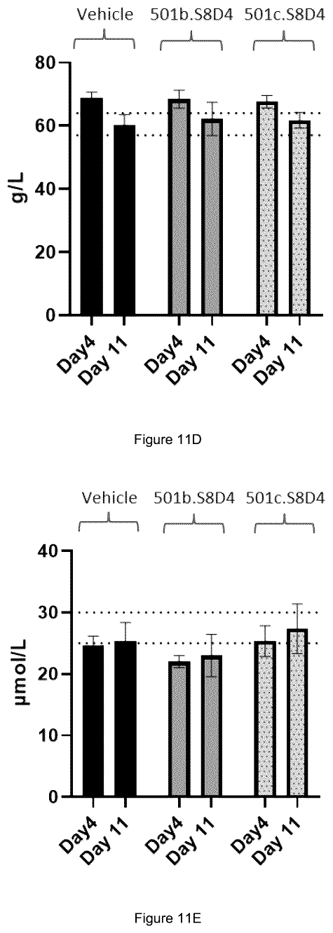

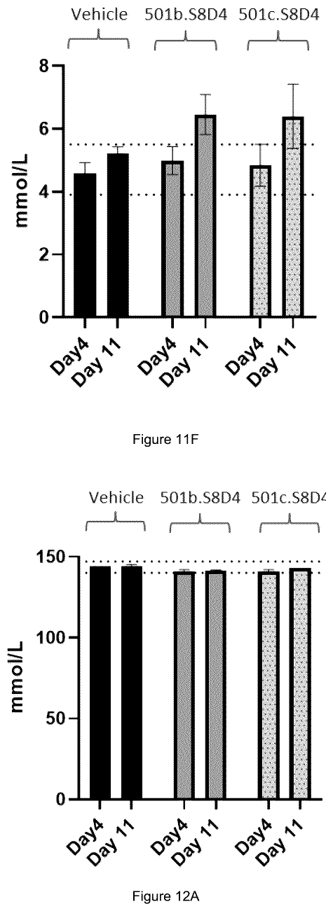

Evaluation of biochemical markers of hepatic, renal and pancreatic damage revealed no significant differences between rats in Groups 1, 2 and 3 (FIGS. 11A to 11F). By contrast, a significant increase in levels of transaminases (markers of liver damage) has previously been detected in preclinical evaluation of Patritumab-DXd (Hashimoto et al., Clin Cancer Res. (2019) 25(23):7151-7161).

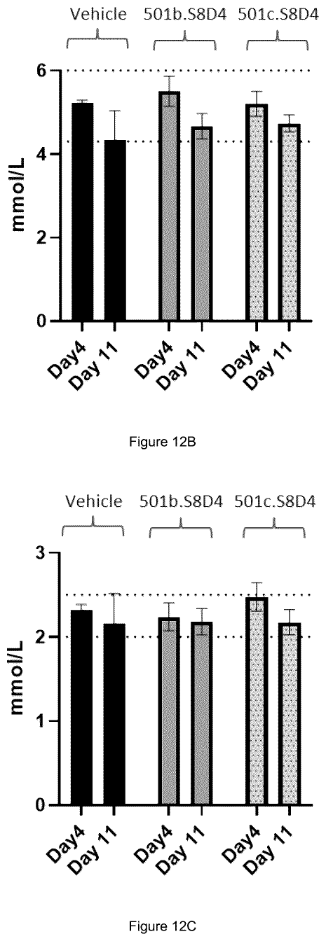

Finally, the levels of various electrolytes were found not to differ significantly between blood samples obtained from rats in Groups 1, 2 and 3 (FIGS. 12A to 12D).

Taken together, the data from the present toxicological studies indicate that 501c.S8D4 and 501b.S8D4 are likely to have improved tolerability as compared to Patritumab-DXd in rats.

1. An antigen-binding molecule that binds to HER3, comprising (i) a HER3-binding moiety, and (ii) a linker-payload moiety comprising exatecan or a derivative thereof.

2. The antigen-binding molecule according to claim 1, wherein the linker-payload moiety comprises structure (A):

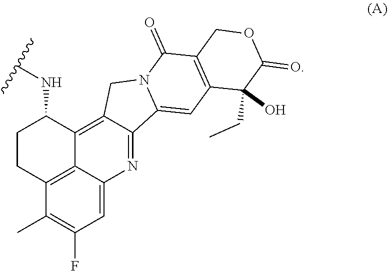

3. The antigen-binding molecule according to claim 1, wherein the linker-payload moiety and the HER3-binding moiety are connected via a cleavable linker moiety.

4. The antigen-binding molecule according to claim 1, wherein the antigen-binding molecule comprises a cleavable linker moiety comprising structure (B):

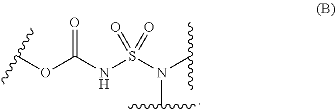 [SMILES: *OC(=O)NS(=O)(=O)N(*)*]

5. The antigen-binding molecule according to claim 1, wherein the antigen-binding molecule comprises a cleavable linker moiety comprising a polyethylene glycol (PEG) moiety.

6. The antigen-binding molecule according to claim 1, wherein the antigen-binding molecule comprises a cleavable linker moiety comprising structure (E):

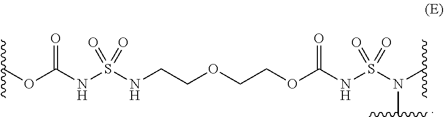 [SMILES: *OC(=O)NS(=O)(=O)NCCOCCOC(=O)NS(=O)(=O)N(*)*]

7. The antigen-binding molecule according to claim 1, wherein antigen-binding molecule comprises a cleavable linker moiety comprising a p-aminobenzyl carbamate (PABC) group.

8. The antigen-binding molecule according to claim 1, wherein the antigen-binding molecule comprises a cleavable linker moiety comprising structure (D): 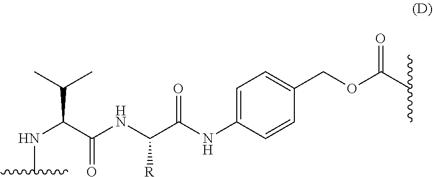
wherein R is selected from CH₃ and (CH₂)₃NHCOCNH₂.

9. The antigen-binding molecule according to claim 1, wherein the antigen-binding molecule comprises structure (E):

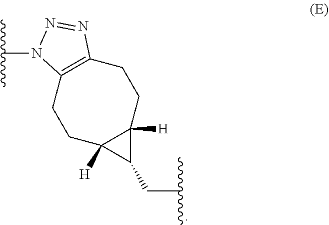 [SMILES: *C[C@H]1[C@H]2CCc3nnn(*)c3CC[C@@H]12]

10. The antigen-binding molecule according to claim 1, wherein the antigen-binding molecule comprises SYNtecan E.

11. The antigen-binding molecule according to claim 1, wherein the HER3-binding moiety comprises an Fc region, and wherein the linker-payload moiety is conjugated to the HER3-binding moiety at an azido group provided in an 6-azido-6-deoxy-N-acetylgalactosamine residue of an N-glycan linked to N297 of a CH2 domain of the Fc region (EU numbering).

12. The antigen-binding molecule according to claim 1, wherein the HER3-binding moiety binds to HER3 in the region shown in SEQ ID NO:77.

13. The antigen-binding molecule according to claim 1, wherein the HER3-binding moiety comprises:
(i) a heavy chain variable (VH) region incorporating the following CDRs:
HC-CDR1 having the amino acid sequence of SEQ ID NO:40
HC-CDR2 having the amino acid sequence of SEQ ID NO:43
HC-CDR3 having the amino acid sequence of SEQ ID NO:48; and
(ii) a light chain variable (VL) region incorporating the following CDRs:
LC-CDR1 having the amino acid sequence of SEQ ID NO:66
LC-CDR2 having the amino acid sequence of SEQ ID NO:69
LC-CDR3 having the amino acid sequence of SEQ ID NO:74.

14. The antigen-binding molecule according to claim 1, wherein the HER3-binding moiety comprises:
(i) a VH region incorporating the following CDRs:
HC-CDR1 having the amino acid sequence of SEQ ID NO:38
HC-CDR2 having the amino acid sequence of SEQ ID NO:42
HC-CDR3 having the amino acid sequence of SEQ ID NO:45; and
(ii) a VL region incorporating the following CDRs:
LC-CDR1 having the amino acid sequence of SEQ ID NO:63
LC-CDR2 having the amino acid sequence of SEQ ID NO:67
LC-CDR3 having the amino acid sequence of SEQ ID NO:70.

15. The antigen-binding molecule according to claim 1, wherein the antigen-binding moiety that binds to HER3 comprises:
a VH region comprising an amino acid sequence having at least 70% sequence identity to the amino acid sequence of SEQ ID NO:33; and
a VL region comprising an amino acid sequence having at least 70% sequence identity to the amino acid sequence of SEQ ID NO:58.

16. The antigen-binding molecule according to claim 1, wherein the antigen-binding moiety that binds to HER3 comprises:
a polypeptide comprising, or consisting of, an amino acid sequence having at least 70% sequence identity to the amino acid sequence of SEQ ID NO:75; and
a polypeptide comprising, or consisting of, an amino acid sequence having at least 70% sequence identity to the amino acid sequence of SEQ ID NO:76.

17. The antigen-binding molecule according to claim 1 to 16, wherein the antigen-binding moiety that binds to HER3 comprises:
a polypeptide comprising, or consisting of, an amino acid sequence having at least 70% sequence identity to the amino acid sequence of SEQ ID NO:80, 81, 82 or 91; and
a polypeptide comprising, or consisting of, an amino acid sequence having at least 70% sequence identity to the amino acid sequence of SEQ ID NO:76.

18. A method for producing an antigen-binding molecule, comprising contacting a HER3-binding moiety comprising an azido moiety with a compound having structure (F):

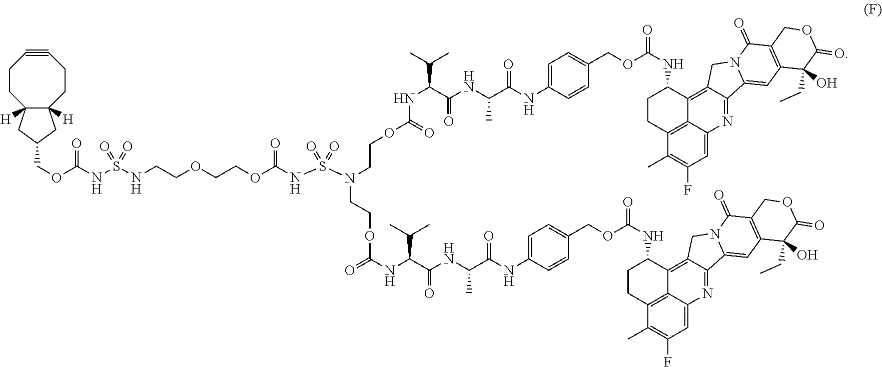 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)OCCN(CCOC(=O)N[C@H](C(=O)N[C@@H](C)C(=O)Nc2ccc(COC(=O)N[C@H]4CCc5c(C)c(F)cc6nc7c(c4c56)Cn4c-7cc5c(c4=O)COC(=O)[C@]5(O)CC)cc2)C(C)C)S(=O)(=O)NC(=O)OCCOCCNS(=O)(=O)NC(=O)OC[C@H]2C[C@H]4CCC#CCC[C@H]4C2)C(C)C)cc1)CC3]

19. An antigen-binding molecule obtained or obtainable by the method of claim 18.

20. A composition comprising an antigen-binding molecule according to claim 1, and a pharmaceutically acceptable carrier, diluent, excipient or adjuvant.

21. An antigen-binding molecule according to claim 1, for use in a method of medical treatment or prophylaxis, or in a method of diagnosis or prognosis.

22. An antigen-binding molecule according to claim 1, for use in treating or preventing a cancer.

23. Use of an antigen-binding molecule according to claim 1, in the manufacture of a medicament for treating or preventing a cancer.

24. A method of treating or preventing a cancer, comprising administering to a subject a therapeutically- or prophylactically-effective amount of an antigen-binding molecule according to claim 1.

25. The method according to claim 24, wherein the cancer is selected from: a cancer comprising cells expressing/overexpressing an EGFR family member, a cancer comprising cells expressing/overexpressing HER3, a cancer comprising cells having a mutation resulting in increased expression of a ligand for HER3, a cancer comprising cells having an NRG gene fusion, a solid tumor, a hematological cancer, a squamous cell cancer, breast cancer, breast carcinoma, breast invasive carcinoma, ductal carcinoma, metastatic breast cancer, triple-negative breast cancer, HER2-positive breast cancer, HER2-negative breast cancer, hormone receptor-positive breast cancer, HER2-negative/hormone receptor-positive breast cancer, gastric cancer, gastric carcinoma, gastric adenocarcinoma, gastrointestinal adenocarcinoma, colorectal cancer, metastatic colorectal cancer, colon cancer, colorectal carcinoma, colorectal adenocarcinoma, colon adenocarcinoma, head and neck cancer, head and neck squamous cell carcinoma (HNSCC), lung cancer, non-small cell lung cancer, lung adenocarcinoma, invasive mucinous lung adenocarcinoma, lung squamous cell carcinoma (LUSC), ovarian cancer, ovarian carcinoma, ovarian serous adenocarcinoma, ovarian serous cystadenocarcinoma, fallopian tube cancer, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, oral cavity cancer, oropharyngeal cancer, esophageal cancer, esophageal squamous cell carcinoma (ESCC), esophageal adenocarcinoma, liver cancer, hepatocellular carcinoma, cholangiocarcinoma, gallbladder cancer, biliary tract cancer, uterine cancer, endometrial cancer, uterine corpus endometrial carcinoma, uterine carcinosarcoma, thyroid cancer, thyroid carcinoma, pheochromocytoma, paraganglioma, bladder cancer, bladder urothelial carcinoma, prostate cancer, prostate adenocarcinoma, castration-resistant prostate cancer, metastatic prostate cancer, metastatic castration-resistant prostate cancer, retinoblastoma, sarcoma, soft tissue sarcoma, peritoneal cancer, thymoma, neuroendocrine tumor, neuroendocrine tumor of the nasopharynx and homologous recombination deficiency (HRD) cancer.

26. Use of an antigen-binding molecule according to claim 1, to deplete or increase killing of cells expressing HER3.

27. An in vitro complex, optionally isolated, comprising an antigen-binding molecule according to claim 1 bound to HER3.

28. A method for detecting HER3 in a sample, comprising contacting a sample containing, or suspected to contain, HER3 with an antigen-binding molecule according to claim 1, and detecting the formation of a complex of the antigen-binding molecule with HER3.

29. A method of selecting or stratifying a subject for treatment with a HER3-targeted agent, the method comprising contacting, in vitro, a sample from the subject with an antigen-binding molecule according to claim 1, and detecting the formation of a complex of the antigen-binding molecule with HER3.

30. Use of an antigen-binding molecule according to claim 1 as an in vitro or in vivo diagnostic or prognostic agent.


SEQUENCE LISTING (100 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 183 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 3 - protein, 331 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 4 - protein, 1283 aa, Homo sapiens
MGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 5 - protein, 699 aa, Homo sapiens
MALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 6 - protein, 1323 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 7 - protein, 164 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 8 - protein, 312 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 9 - protein, 624 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 10 - protein, 21 aa, Homo sapiens
MALTVIAGLVVIFMMLGGTFL

SEQ ID NO: 11 - protein, 678 aa, Homo sapiens
YWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 12 - protein, 44 aa, Homo sapiens
YWRGRRIQNKRAMRRYLERGESIEPLDPSEKANKVLARIFKETE

SEQ ID NO: 13 - protein, 258 aa, Homo sapiens
LRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFT

SEQ ID NO: 14 - protein, 376 aa, Homo sapiens
RMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 15 - protein, 164 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSC

SEQ ID NO: 16 - protein, 146 aa, Homo sapiens
PPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPK

SEQ ID NO: 17 - protein, 166 aa, Homo sapiens
ACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVA

SEQ ID NO: 18 - protein, 148 aa, Homo sapiens
EGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 19 - protein, 17 aa, Homo sapiens
QPLVYNKLTFQLEPNPH

SEQ ID NO: 20 - protein, 1283 aa, Macaca mulatta
MGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWKDIVRDQDAEIVVKDNGRSCPLCHEVCKGRCWGPGPEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMYNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKIIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGEAFQESAVSGSSEWCPRPVSLHPMPRGCLASESSEGHVTGSEAELQEKVSTCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPRPPRPSSLEELGYEYMDVGSDLSASLGSTQSCPLHPVPVMPTAGTTPDEDYEYMNRQRGGSGPGGDYAAMGACPASEQGYEEMRAFQGPGHQAPHVHYAHLKTLRSLEATDSAFDNPDYWHSRLFPKANAQRT

SEQ ID NO: 21 - protein, 120 aa, synthetic construct
DVQLQESGPDLVKPSQSLSLTCTVTGYSITSGYSWHWIRQFPGNKLEWMGSIHYSGGTNYNPSLKSRISITRDTSKNQFFLQLNSVTTEDTATYFCARMTTAPRYPFDYWGQGTTLTVSS

SEQ ID NO: 22 - protein, 120 aa, synthetic construct
DVQLQEWGAGLLKPSETLSLTCAVTGYSITSGYSWHWIRQFPGNGLEWIGSIHYSGGTNYNPTLKSRITISRDTSKNQFSLKLSSVTAADTAVYFCARMTTAPRYPFDYWGQGTLVTVSS

SEQ ID NO: 23 - protein, 120 aa, synthetic construct
DVQLQEWGAGLLKPSETLSLTCAVTGYSITSGYSWHWIRQFPGNGLEWIGSIHYSGGTNYNPSLKSRITISRDTSKNQFSLKLSSVTAADTAVYFCARMTTAPRYPFDYWGQGTLVTVSS

SEQ ID NO: 24 - protein, 120 aa, synthetic construct; identical to SEQ ID NO: 23

SEQ ID NO: 25 - protein, 120 aa, synthetic construct; identical to SEQ ID NO: 23

SEQ ID NO: 26 - protein, 120 aa, synthetic construct
DVQLQEWGAGLLKPSETLSLTCAVTGYSITSGYSWHWIRQFPGKGLEWIGSIHYSGGTNYNPSLKSRITISRDTSKNQFSLKLSSVTAADTAVYFCARMTTAPRYPFDYWGQGTLVTVSS

SEQ ID NO: 27 - protein, 120 aa, synthetic construct
DVQLQEWGAGLLKPSETLSLTCAVYGYSITSGYSWHWIRQPPGKGLEWIGSIHYSGGTNYNPSLKSRVTISRDTSKNQFSLKLSSVTAADTAVYYCARMTTAPRYPFDYWGQGTLVTVSS

SEQ ID NO: 28 - protein, 120 aa, synthetic construct
DVQLQEWGAGLLKPSETLSLTCAVTGYSITSGYSWHWIRQFPGNGLEWIGSIRYSGGTNYNPSLKSRITISRDTSKNQFSLKLGSVTAADTAVYFCARMTTAPRYPFDYWGQGTLVTVSS

SEQ ID NO: 29 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARMTTAPRYPFDYWGQGTLVTVSS

SEQ ID NO: 30 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARETTAPRYPFDYWGQGTLVTVSS

SEQ ID NO: 31 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARGTTAPRYPFDYWGQGTLVTVSS

SEQ ID NO: 32 - protein, 120 aa, synthetic construct
DVQLQEWGAGLLKPSETLSLTCAVTGYSITSGYSWHWIRQFPGNGLEWIGSIHYSGGTNYNPSLKSRITISRDTSKNQFSLRLSSVTAADTAVYFCARMTTAPRYPFDYWGQGTLVTVSS

SEQ ID NO: 33 - protein, 120 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSS

SEQ ID NO: 34 - protein, 120 aa, synthetic construct
QVQLQESGPGLVKPSQTLFLTCTVSGYSITSGYSWHWIRQHPGKGLEWIGSIRYSGGTDYNPSLKSLVTISVDTSKNQFSLKLSSVTAADTAVYYCARMTTAPWYPFDYWGQGTTVTVSS

SEQ ID NO: 35 - protein, 120 aa, synthetic construct
QVQLQESGPGLVKPSQTLSLTCTVSGYYITSGYSWHWIRQHPGKGLEWIGSIRYSGGTDYNPSLKSLATISADTSKNQFSLKLSSVTAADTAVYYCARMTTAPWYPFDYWGQGTAVTVSS

SEQ ID NO: 36 - protein, 120 aa, synthetic construct; identical to SEQ ID NO: 22

SEQ ID NO: 37 - protein, 120 aa, synthetic construct; identical to SEQ ID NO: 32

SEQ ID NO: 38 - protein, 9 aa, synthetic construct
GYSITSGYS

SEQ ID NO: 39 - protein, 9 aa, synthetic construct
GYYITSGYS

SEQ ID NO: 40 - protein, 9 aa, synthetic construct
  VARIANT 3: note = X = S or Y
GYXITSGYS

SEQ ID NO: 41 - protein, 7 aa, synthetic construct
IHYSGGT

SEQ ID NO: 42 - protein, 7 aa, synthetic construct
IRYSGGT

SEQ ID NO: 43 - protein, 7 aa, synthetic construct
  VARIANT 2: note = X = H or R
IXYSGGT

SEQ ID NO: 44 - protein, 13 aa, synthetic construct
ARMTTAPRYPFDY

SEQ ID NO: 45 - protein, 13 aa, synthetic construct
ARMTTAPWYPFDY

SEQ ID NO: 46 - protein, 13 aa, synthetic construct
ARETTAPRYPFDY

SEQ ID NO: 47 - protein, 13 aa, synthetic construct
ARGTTAPRYPFDY

SEQ ID NO: 48 - protein, 13 aa, synthetic construct
  VARIANT 3: note = X = M, E, or G
  VARIANT 8: note = X = R or W
ARXTTAPXYPFDY

SEQ ID NO: 49 - protein, 107 aa, synthetic construct
DIVMTQSQKFMSTSVGDRVSVTCKASQIVGSNVAWYQQKPGQSPKPLIYSASYRYSGVPDRFTASGSGTDFTLTITNVQSEDLAEYFCQQYSSHPLTFGAGTKLELK

SEQ ID NO: 50 - protein, 107 aa, synthetic construct
DIVMTQSPSSLSASVGDLVTITCKASQIVGSNVAWYQMKPGKSPKPLIYSASYLYFGVPSRFSGSGSGTDFTLTISSLQPEDVAEYFCQQYSSHPLTFGPGTKVEIK

SEQ ID NO: 51 - protein, 107 aa, synthetic construct
DIVMTQSPSSLSASGGDRVTITCKASQIVGYNVAWYQQKPGKSPKPLIYSASYLYSDVPSRFSASGSGTDFTLTISSLQPEDVAEYFCQQYSSHPLTFGPGTKVEIK

SEQ ID NO: 52 - protein, 107 aa, synthetic construct
VIVMTQSPSSLSASVGDRVTITCKASQIVGPNVAWYQQKPGKSPKPLIYSASYGYSDVPSRFSGSGSGTDFTLTISSLQPEDVAEYFCQQYSTHPLTFGPGTKVEIK

SEQ ID NO: 53 - protein, 107 aa, synthetic construct
DIVMTQSPSSLSASVGDRVTITCKASQIVGSNVAWYQQKPGKSPKPLIYSASYGYSDVPSRFSGSGSGTDFTLTISSLRPEDVATYYCQQYSSHPLTFGPGTKVEIK

SEQ ID NO: 54 - protein, 107 aa, synthetic construct
DIVMTQSPSSLSASVGDRVTITCKASQIVGSNVAWYQQKPGKSPKPLIYSARYQYSGVPFRFSGSGSGTDFTLTISSLQPEDVATYYCQQYSSHPLTFGPGTKVEIK

SEQ ID NO: 55 - protein, 107 aa, synthetic construct; identical to SEQ ID NO: 54

SEQ ID NO: 56 - protein, 107 aa, synthetic construct; identical to SEQ ID NO: 54

SEQ ID NO: 57 - protein, 107 aa, synthetic construct
DIVMTQSPSSLSASVGDRVTITCKASQIVGSNVAWYQQMPGKSPEPLIYSASYLYSDVPSRFSGSGSGTDFTMTISSLQPEDVATYYCQQYSSHPLTFGPGTKVEIK

SEQ ID NO: 58 - protein, 107 aa, synthetic construct
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIK

SEQ ID NO: 59 - protein, 107 aa, synthetic construct
DIQMTQSPSSVSASVGDRVTFTCKASQIVGSNVAWYQQKPGKAPEPLIYSASYLYSSVPSRFSGSGSGTEFTMTISSLEPEDFATYYCQQYTTHPLTFGPGTKVEIK

SEQ ID NO: 60 - protein, 107 aa, synthetic construct
DIQMTQSPSSVSASVGDRVTITCKASQIVGSNVAWYQQKPGKAPMPLIYSASYGYSGVPSRFSGSGSGTDFTLTISSLQPEDFATYYCQQYSSHPLTFGQGTKLEIK

SEQ ID NO: 61 - protein, 107 aa, synthetic construct
DIVMTQSPSSLSASVGDLVTITCKASQIVGSNVAWYQMKLGKSPKPLIYSASYLYFGVPSRFSGSGSGTDFTLTISSLQPEDVAEYFCQQYFSHPLTFGPGTKVEIK

SEQ ID NO: 62 - protein, 107 aa, synthetic construct
DIVMTQSPSSLSASVGDRVTITCKASQIVGSNVAWYQQKPGKSPKPLIYSASYLYSDVPSRFSGSGSGTDFTMTISSLQPEDVATYYCQQYSSHPLTFGPGTKVEIK

SEQ ID NO: 63 - protein, 6 aa, synthetic construct
QIVGSN

SEQ ID NO: 64 - protein, 6 aa, synthetic construct
QIVGYN

SEQ ID NO: 65 - protein, 6 aa, synthetic construct
QIVGPN

SEQ ID NO: 66 - protein, 6 aa, synthetic construct
  VARIANT 5: note = X = S, Y, or P
QIVGXN

SEQ ID NO: 67-69 - skipped (3 entries)

SEQ ID NO: 70 - protein, 9 aa, synthetic construct
QQYSSHPLT

SEQ ID NO: 71 - protein, 9 aa, synthetic construct
QQYSTHPLT

SEQ ID NO: 72 - protein, 9 aa, synthetic construct
QQYTTHPLT

SEQ ID NO: 73 - protein, 9 aa, synthetic construct
QQYFSHPLT

SEQ ID NO: 74 - protein, 9 aa, synthetic construct
  VARIANT 4: note = X = S, T, or F
  VARIANT 5: note = X = S or T
QQYXXHPLT

SEQ ID NO: 75 - protein, 450 aa, synthetic construct
  VARIANT 237: note = X = L or A
  VARIANT 238: note = X = L or A
  VARIANT 255: note = X = M or Y
  VARIANT 257: note = X = S or T
  VARIANT 259: note = X = T or E
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEXXGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 214 aa, synthetic construct
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 18 aa, synthetic construct
CFGPNPNQCCHDECAGGC

SEQ ID NO: 78 - protein, 5 aa, synthetic construct
PNPNQ

SEQ ID NO: 79 - protein, 5 aa, synthetic construct
DECAG

SEQ ID NO: 80 - protein, 450 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 450 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 450 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 86 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 87 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 217 aa, Homo sapiens
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 217 aa, Homo sapiens
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 217 aa, Homo sapiens
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 450 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 220 aa, synthetic construct; identical to SEQ ID NO: 93

SEQ ID NO: 98 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 220 aa, synthetic construct; identical to SEQ ID NO: 93

SEQ ID NO: 100 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK